(19) United States
(12) Patent Application Publication
(10) Pub. No.: US 2003/0054389 A1
(43) Pub. Date: Mar. 20, 2003

(54) Methods for identifying agents that induce a cellular phenotype, and compositions thereof

(76) Inventor: Carl Alexander Kamb, Salt Lake City, UT (US)

Correspondence Address: MARSHALL, GERSTEIN & BORUN, 6300 SEARS TOWER, 233 SOUTH WACKER, CHICAGO, IL, 60606-6357, US

(21) Appl. No.: 10/196,408

(22) Filed: Jul. 16, 2002

Related U.S. Application Data
(60) Provisional application No. 60/309,088, filed on Jul. 31, 2001.
(60) Provisional application No. 60/305,711, filed on Jul. 16, 2001.
(60) Provisional application No. 60/305,712, filed on Jul. 16, 2001.

(51) Int. Cl. C12Q 1/68; G01N 33/574

(52) U.S. Cl. 435/6; 435/7.23

(57) ABSTRACT

The present invention is directed to methods for performing negative selection assays leading to the identification of cytostatic or cytotoxic agents that cause a lethal phenotype. The invention is useful also for evaluation of conditional cytotoxicity and cell-specific cytotoxicity.

### RELATED APPLICATIONS

This application claims priority of U.S. Provisional Application Serial No. 60/309,088, filed Jul. 31, 2001, U.S. Provisional Application Serial No. 60/305,711, filed Jul. 16, 2001, U.S. Provisional Application Serial No. 60/305,712, filed Jul. 16, 2001, and U.S. patent application Ser. No. 09/504,123, filed Feb. 15, 2000, which is pending, the entire disclosures of each are incorporated herein by reference.

### FIELD OF THE INVENTION

The present invention generally relates to cancer therapy and more particularly, to methods and compositions relating to identifying agents that induce a desired cellular phenotype, such as cell death, cell differentiation, and cell proliferation.

### BACKGROUND OF THE INVENTION

Cancer and other diseases involving abnormal or undesirable cell proliferation or differentiation processes that lead to the adaptation of altered or modified cellular traits present a major challenge to the pharmaceutical industry. Desirable therapeutic compounds bind to diseased cellular targets and inhibit cell growth (or kill unwanted cells) without effecting normal/wildtype cells. Unfortunately, such cellular targets are difficult to identify because cells exhibiting the desired phenotype (e.g. cell death) disappear from the population during the screening procedure and consequently the targets (and corresponding causative agents) are lost.

Negative Selection Assays. Experiments that identify agents that inhibit cell growth and/or kill cells by necrotic or apoptotic means are termed negative selections, i.e. selections for compounds that exert a cytotoxic or cytostatic effect on a cell population. In general, desirable negative selections embody a variety of properties including, but not limited to: i) employment of simple procedures for identifying dead and/or dying cells; ii) utilization of an open-ended strategy or design, i.e. using a “black-box” approach that enables the identification of new drug targets, and; iii) identification of both the drug (or agent) and the target molecule on which the drug acts.

In one example, cells grown in a high-density format are exposed to one or more members of a chemical library and wells containing high levels of cell death are correlated with unique library members. While this procedure may be a fast and productive means for identifying new drug candidates, target elucidation is often complex and elusive. In a second procedure, protein targets that are already known to play a key role in a given disease pathway are first screened for agents that have affinity for the known target using various binding or displacement assays. Subsequently, these agents are tested in one or more bioassays for cytotoxic effects.

The techniques described above are inherently limited by the scope of pre-existing knowledge of such key proteins. To maximize the development of new chemotherapeutic agents for the treatment of diseases, it is advantageous to be able to broadly and generally screen for cytotoxic compounds without being so limited to a small pre-existing pool of targets.

High throughput screening. High throughput screens have been achieved in several chemical and biological assays. For instance, microarrays of EST's sequences (e.g. DNA chips, GENECHIP™). In these procedures, thousands of individual DNA sequences are attached to defined locations on a small glass plate, (U.S. Pat. No. 5,556,752) and hybridized with fluorescently labeled, complementary DNA (cDNA) sequences obtained from, for instance, normal or malignant tissue. Through these procedures, researchers are able to screen through tens of thousands of expressed sequences on a single slide and identify key changes in a cell's physiology over the course of disease advancement.

In a similar fashion, protein sequences have been screened by high throughput techniques. For example, protein microarrays similar to the DNA arrays described above have been constructed on glass plates and screened to identify peptides capable of, for instance, binding receptors.

In addition to non-living arrays such as the cDNA and peptide screens described above, high throughput screens that analyze living cells have also been developed. Unlike non-living arrays, whole-cell high throughput screens have the ability to test potential drug therapeutics in an intact cell format. As a result of this arrangement, specific phenotypes (e.g. differentiation, cell death, proliferation) based on more complex biological activities including those involving multisubunit complexes and/or multistep pathways, can be screened. Few high throughput methods, however, exist for screening cDNA or synthetic peptide expression libraries for agents capable of inducing i) transcriptional activation of correlative reporter genes, ii) viral resistance, or iii) cell growth arrest or cell death.

Additionally, a difficulty suffered by many other live-cell genomic screens is the inefficiency by which individual clones carrying presumptive drug candidates are re-tested for the phenotype of interest. Because the size of the genetic expression libraries used in these methods can be considerable (>10⁶ clones), retesting individual members of a subpool containing, for instance, 0.1% of the original library (e.g. thousands of candidate molecules) can be cumbersome and cost ineffective. For this reason, a need exists for the development of high-throughput technologies capable of testing a large number of clones in a transdominant genetic format.

Accordingly, a need for rapid and more efficient methods of identifying agents that induce a desired phenotype, including, for example, cell differentiation, cell death, and cell proliferation, exists in the art. Such efficient and rapid methods would further allow for the identification of potential drugs for cancer therapy and cellular targets useful in the treatment of diseases.

### SUMMARY OF THE INVENTION

The present invention generally relates to methods and compositions directed to identifying and screening for agents that induce a cellular phenotype. More particularly, the present invention provides rapid and efficient methods for identifying and screening for agents that induce a cellular phenotype such as cell death, cell differentiation, and cell proliferation.

The present invention provide methods to identify a candidate compound that inhibits cell proliferation comprising the steps of: a) introducing into a cell population a polynucleotide library under conditions that permit expression of polypeptides and/or ribonucleic acid encoded by the library polynucleotides; b) isolating cells in the population in which proliferation is inhibited; and c) isolating the polynucleotide library sequence(s) from cells detected in step (b), wherein inhibited proliferation of the cell identifies the encoded polypeptide or ribonucleic acid as a candidate compound that inhibits cell proliferation. Methods of the invention are used to identify candidate compounds in proliferation-inhibited cells that are dead and/or dying, and in one aspect, the cells are apoptotic. In another aspect, the proliferation inhibited cells are growth arrested. In another aspect, the proliferation inhibited cells are identified by a loss of adherence to a solid support.

Methods of the invention also include identification of proliferation inhibited cells using an agent that recognizes and binds an intracellular component that is accessible in dead and/or dying cells or is activated during cell cycle arrest or programmed cell death. In one aspect, the agent is a labeling compound, and in various aspects of the invention, the labeling compound binds a membrane lipid, is a DNA affinity dye, or an antibody, wherein the antibody is immunospecific for an intracellular antigen. In one aspect, the antibody is detectably labeled.

Methods of the invention also embrace identification and isolation of proliferation inhibited cells using fluorescent activated cell sorting (FACS).

The invention also provides methods which are fully and/or partially automated.

In accordance with certain aspects of the present invention, methods for performing negative selections are provided. In accordance with one aspect, the negative selections are performed by introducing a genetic library into a population of target cells, collecting a subpopulation of cells that detach from a culturing surface (referred to herein as “floaters”) and then recovering the introduced genetic material from that subpopulation. In another aspect, the methods involve introducing a genetic library into a population of target cells, identifying cells that develop permeable membranes, and then recovering the transformed or transduced genetic material from that subpopulation. Such methods can be performed manually or in a roboticized, high-throughput format.

In a further aspect of the present invention, cell-specific cytotoxic agents are identified by employing a counter-screening step wherein the genetic material from the subpopulation displaying a detachment, a membrane permeable, and/or other measures of a lethal phenotype is introduced into a second, different population of cells, and a second sublibrary of genetic material is obtained from a second subpopulation that does not display detachment and/or the lethal phenotype. For instance, cDNA sequences that induce cell death in i.e. a cancer cell line might be counter-screened against one or more normal cell types to identify cDNAs that kill the diseased cell type, but not the normal cell type. In still other embodiments, the invention provides methods for recovering agents that induce a lethal phenotype from dead and/or dying cells.

In certain embodiments of the present invention, the lethal phenotype of the methodology may be apoptosis, necrosis, or growth arrest. In embodiments in which the lethal phenotype is apoptosis, the property of disattachment from a culturing substrate or membrane permeability may be used as a surrogate for apoptosis, thereby providing a technique for enriching the apoptotic cell population. In other particular embodiments, the genetic material may be partially sequenced, or the method steps may be reiterated in a second population of the same cells to further enrich for desirable cytotoxic/cytostatic sequences. The target cells may be any mammalian cells, or more particularly primary cells, especially primary cells derived from epithelial or endothelial cells, stem cells, mesenchymal cells, fibroblasts, neuronal cells or hematopoeitic cells. The mammalian cells may also be cancer cells, or more particularly cancer cells that are metastatic, derived from solid tumors, or cultured cell lines. The cancer cells may particularly be derived from breast, colon, lung, melanoma or prostate tissue. In other particular embodiments, the mammalian cells are genetically altered, and more particularly may be immortalized or transformed.

In embodiments that utilize the property of disattachment of target cells from a culturing surface, particular embodiments will feature a low background of spontaneously detaching cells, which may more particularly be no more than about 10%, or alternatively no more than about 2%. Target cells having such low backgrounds include SW620 and HT29 colon cancer cells, T47D breast cancer cells, HuVEC cells, and others. In particular embodiments, the unadhering cells are collected over a period of at least about 12 hours.

The invention also lends itself to embodiments that screen for conditional cytotoxicity (as defined herein) wherein a genetic library is introduced into a population of target cells, exposing those target cells to a subtoxic threshold dose of a secondary reagent, collecting a subpopulation of cells displaying a lethal phenotype, and recovering genetic material from that subpopulation. Again, in particular embodiments the lethal phenotype may be apoptosis, necrosis or growth arrest. In other particular embodiments, the secondary reagent may be UV, X-ray or neutron radiation, or may be a chemotherapeutic agent. Particular embodiments include cancer cells, more particularly solid tumors, as target cells, counterscreening with a second cytotoxic substance, preconditioning the target cells prior to exposure with, e.g., growth factors, cytokines, chemokines, or activation of oncogenes. In an additional embodiment the assays described above can be performed in a high-throughput procedure whereby robotic elements are utilized to screen through thousands, or tens of thousands of potential cytotoxic agents for those sequences that induce cell death and/or cytostasis in the host cell of choice.

The invention also describes the criteria by which all compositions of matter isolated from such a screen are defined. These parameters include, but are not limited to, chemical makeup, size, fragments of such agents, binding properties, coding sequences, and more. The invention also lends itself to embodiments that define the construction or use of genetic libraries. Such libraries can be derived from natural sources such as genomic DNA or cDNA taken from human, mouse, nematode, fly, yeast, or other organisms, or they may be synthetically constructed using art-proven technologies.

The invention also encompasses the identification of small organic molecules that induce a lethal phenotype. In some embodiments, organic molecules that displace a proteinaceous cytotoxic agent from an endogenous protein target are obtained. In other embodiments, organic molecules having a structure-activity relationship with that proteinaceous cytotoxic agent are identified.

The present invention also provides methods for identifying a cellular phenotype by using high throughput screening (HTS) procedures. Within certain aspects, the methods of the present invention provide for the screening of genetic expression libraries for agents that induce unique cellular phenotypes.

In one aspect, genetic expression libraries are introduced into somatic cells and screened for relevant phenotypes. In accordance with this aspect, the cellular phenotypes include cell death, cell proliferation, transcriptional activation of pertinent reporter construct(s), or resistance to pathogen infection such as viruses. Cells expressing the phenotype of interest are collected by one of several means (e.g. panning, FACS) and subsequently re-screened to enrich the population for molecules that induce the desired trait.

In another aspect, agents (also referred to herein as “perturbagens”) that induce a cellular phenotype can consist of polypeptides or nucleic acids, and may be encoded by a naturally derived library of compounds such as a cDNA or genomic DNA (gDNA) expression library, or an artificial library comprising synthetic oligonucleotide sequences of a desired length or range of lengths, e.g. a random peptide library. Such agents are capable of, disrupting, activating, or modulating a particular signaling pathway and/or cellular event.

### BRIEF DESCRIPTION OF THE DRAWINGS

FIG. 1. Floater Screen. HT29 adenocarcinoma cells transfected with a perturbagen library are cultured for 3-5 days. Subsequently, floating cells are centrifuged, processed for genomic DNA, and PCR amplified to retrieve perturbagen-encoding sequences. As one alternative to these procedures, floating cells can be stained with propidium iodide (PI) and sorted for PI+ (dead) cells. The PCR product is then ligated into a retroviral vector, packaged, and reintroduced into a naïve population of HT29 cells for additional rounds of screening and enrichment.

FIG. 2. Perturbagen Disruption of Macromolecular Structures. Assembly of macromolecular subunits into stable quaternary structures requires the interaction between critical epitopes of the participating macromolecules. For instance, to maintain a helical structure composed of two heterologous subunits (α and β), α-α, β-β and α-β interactions must occur. Although many peptides can be found which show affinity to α or β subunits, most do not disrupt macromolecular assembly. One method of action, a perturbagen can bind to a critical epitope and disrupt the association of the two interacting proteins. In this example, a small peptide (represented by the black triangle) binds to the α subunit and disrupts the interaction between α and β subunits. As a result, the helix is disrupted.

FIG. 3. A. Mapping the Biologically Important Region of a Perturbagen. Four perturbagens are derived from different breakpoints within the same gene. By mapping the smallest sequence that is common to all four perturbagens (dotted line) it is possible to identify biologically critical regions (black box). B. Critical regions of a gene can be determined by deletion analysis. For instance, a series of N-terminal deletions (dotted line) can be tested for biological activity. In this example, full activity requires a molecule that is longer than deletion 2 but smaller than deletion 1.

FIG. 4. Basic Two-Hybrid Methodology. When bait and prey molecules interact, the Ga14-AD and Ga140-BD binding domains of the Ga14 transcriptional activator are reconstituted. As a result, this functional unit can associate with the Gal1 UAS and induce transcription of the reporter gene (Leu2).

FIG. 5 is a bar graph depicting the results of FACS analysis of Jurkat cells labeled with Apo2.7, in response to induction of apoptosis with the anti-FAS antibody.

FIG. 6 is a pair of histograms depicting the differential fluorescence patterns of adherent vs. disadhered (“floater”) cells stained with propidium iodide.

FIG. 7 is the FACS analysis of uninfected and mock-infected HT29 cells. The mock-infected cells contain a GFP marker.

FIG. 8 is the FACS histogram depicting differential patterns of PI staining in floater vs. adherent cell populations, 24 hours after exposure to PI.

FIG. 9 is a diagrammatic representation of the construction of a GFP reporter vector having internal XhoI/EcoRI/BamHI restriction sites. Two sets of primers were used to PCR amplify the left- and right-hand segments of GFP. The internal primer of each primer set contains either XhoI-EcoRI or EcoRI-BamHI restriction sites, as indicated. The subsequent digest (EcoRI) and ligation of these fragments recreates GFP with a new internal cloning site, XhoI-EcoRI-BamHI. Subsequent PCR amplification with the two external primers allows amplification of the new GFP.

FIG. 10 is a FACS histogram of PI+ (dead) HT29 cells (gate M1), and a gel showing subsequent PCR amplification of that fraction.

FIG. 11 is a gel comparing the PCR amplification of apoptotic cells, live cells and gDNA controls.

FIG. 12 depicts the analysis of clones from Sort VI of the HT29 floater assay described herein. Thirty six clones picked at random were tested in the HT29 floater assay. Five clones (01, 02, 03, 04, and 05) showed increased levels of floaters that were statistically significant relative to background.

FIG. 13 is a bar graph depicting the floater rates in SW620 cells at F0 (starting library), F2 (after one collection and one sort), F3 (after one collection and two sorts) and F4 (after one collection, three sorts), wherein SW620 cells were infected with the random peptide perturbagen library and taken through several cycles of the negative selection described herein. Floater rate percentages were calculated at each step and compared with mock infected and pVT334 infected controls.

FIG. 14 is a bar graph depicting cell number observations for the SW620 cells of the negative selection described herein. Equal numbers of control (i.e., mock infected and pVT334-infected) cells and F3 (peptide library infected) cells were plated in T75 flasks. On day 5, the flasks were washed and trypsinized and the total number of adherent cells was determined.

FIG. 15 is a kill curve for varying amounts of camptothecin in T47D cells.

FIG. 16a, b, shows two graphs comparing the doubling time and senescence of two HuVEC cell isolates, 8F1868 and 9F0293.

FIG. 17a, b, c, is a set of bar graphs showing the effects of retroviral infection on cell number, doubling time and floater rate. HuVEC 9F0293 cells were infected with the pLIBEGFP vector and studied to determine the effects of retroviral infection and infection procedures on cell number, doubling time and floater rates. Doubling time is measured in hours. Floater rates are measured in percentages (number of floating cells/number of adherent cells).

FIG. 18 is a FACS histogram depicting the time course of PI−/PI+ HuVECs in puromycin-treated cultures. 9F0293 cells were treated with puromycin (2 μg/ml) and followed over the course of 24 hours. Floater cells were collected at defined intervals and were treated with PI and subjected to FACS analysis to determine the percentage of dead and/or dying cells.

FIG. 19 is a diagrammatic representation of the P-glycoprotein pump-mediated extrusion of Rhodamine 123 from an MDR1 cell, in the presence and absence of disruptive library inserts. In an untransformed MDR1 cell, the P-glycoprotein actively pumps Rh123 out of the cell, causing the cells to be “dim.” In the MDR1 cell bearing an agent that disrupts the pumps action, the function of P-glycoprotein is blocked and as a result, the cells retain Rh123 and remain “bright.”

FIG. 20. Diagram of the retroviral vector, pVT340.

FIG. 21. Floater Enrichment. Bar graph showing the increasing numbers of floaters over the course of seven cycles of enrichment.

FIG. 22. Kill Indexes of Perturbagens in HT29 and SW620 Cells

FIG. 23. Kill Indexes of Perturbagens In-Frame (IF) and Out-Of-Frame (OF).

FIG. 24. Bar Graph comparing the cytotoxic effects of Clone 3 in HT29, 96C, HuVEC, and HMEC cell lines.

FIG. 25. Histograms of cells containing a) TBE2EGFP, b) TBE2EGFP+an activated form of catenin (S4535), and c) S4535+a dominant negative regulator of the pathway, dGFPΔTcf4DI.

Definitions

The terms “perturbagen” or “phenotypic probe” as used herein refer to an agent that is proteinaceous or ribonucleic in nature and acts in a transdominant mode to interfere with specific biochemical processes in cells, i.e., through its interaction with specific cellular target(s) or other such component(s), capable of disrupting, activating, or modifying a particular signaling pathway and/or cellular event. Perturbagens may be encoded by a naturally derived library of compounds such as a cDNA or genomic DNA (gDNA) expression library, or an artificial library comprising synthetic oligonucleotide sequences of a desired length or range of lengths, e.g. a random peptide library. Alternatively, the perturbagen itself can be synthesized using chemical methods. The term “proteinaceous perturbagen” encompasses peptides, oligo- or polypeptides, proteins, protein fragments, or protein variants. Some proteinaceous perturbagens can be as short as three amino acids in length. Alternatively, these agents can be greater than 3 amino acids but less than ten amino acids. Other agents can be greater than ten amino acids but shorter than 30 amino acids in length. Still other agents can be greater than 30 amino acids but less than 100 amino acids in length. Still other agents can be greater than 100 amino acids in length. Naturally occurring proteinaceous perturbagens (i.e. those derived from cDNA or genomic DNA) exhibit a range in size from as little as three to several hundred amino acids. In contrast, synthetic perturbagens (such as those present in a synthetic peptide library) may range in size from three amino acids to fifty amino acids in length and more preferably, from three to 20 amino acids in length, and yet more preferably, about 15 amino acids in length.

The term “mimetic” refers to a small molecule that (i) exerts the same or similar physiological or phenotypic effect in a bioassay system or in an animal model as does a given perturbagen, or (ii) is capable of displacing a perturbagen from a target in a displacement assay.

The term “small molecule,” as used herein, refers to a chemical compound, for instance a peptidometic or oligonucleotide that may optionally be derivatized, or any other low molecular weight organic compound, either natural or synthetic. Such small molecules may be a therapeutically deliverable substance or may be further derivatized to facilitate delivery.

The term “target” refers to any host cellular component or foreign cellular component (such as a protein or RNA molecule encoded by a pathogen) that is directly acted upon by the perturbagen that leads to and/or induces the phenotypic change, detectible for example in a bioassay system.

The terms “library” or “genetic library” refer to a collection of nucleic acid fragments that may individually range in size from about a few base pairs to about a million base pairs.

These fragments are generated using a variety of techniques familiar to the art.

The term “sublibrary” refers to a portion of a genetic library that has been isolated by application of a specific screening or selection procedure.

The term “insert” in the context of a library refers to an individual DNA fragment that constitutes a single member of the library.

By “negative selection” is meant a procedure designed to identify and isolate cells that are in one of any number of stages of growth arrest and/or cell death—i.e., are evidencing a lethal phenotype. “Lethal phenotype” is defined as one or more cellular events that result, directly or indirectly, in death of an individual cell or a cell population.

The terms “reporter gene” and “reporter” refer to nucleic acid sequences (or encoded polypeptides) for which screens or selections can be devised. Reporters may be proteins capable of emitting light, embody enzymatic activities, or be genes that encode intracellular or cell surface proteins detectible by antibodies. Preferably, the reporter activity may be evaluated in a quantitative manner. Alternatively, reporter genes can confer antibiotic resistance or gene products necessary for growth in the absence of particular nutrients.

The terms “operably-associated” and/or “operably-linked” refer to functionally related nucleic acids. A promoter is “operably-associated” or “operably-linked” with a coding sequence if the promoter or element controls or regulates the transcription of the gene to which it is linked.

The term “gene” refers to a DNA substantially encoding an endogenous cellular component, and includes both the coding and antisense strands, the 5′ and 3′ regions that are not transcribed but serve as transcriptional control domains, and transcribed but not expressed domains such as introns (including splice junctions), polyadenylation signals, ribosomal recognition domains, and the like.

The terms “polynucleotide” or “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-stranded, double-stranded and triple helical molecules.

“Oligonucleotide” refers to polynucleotides of between 5 and about 100 nucleotides of single- or double-stranded DNA. Oligonucleotides are also known as oligomers or oligos and may be isolated from genes, or chemically synthesized by methods known in the art. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

The term “fragment” refers to any portion of a proteinaceous perturbagen that is at least 3 amino acids in length, or any RNA molecule that is at least 5 nucleotides in length. The descriptors “biologically relevant” or “biologically active” refer to that portion of a protein or protein fragment, RNA or RNA fragment, or DNA fragment that encodes either of the two previous entities, that is responsible for an observable phenotype (or for activation of a correlative reporter construct).

The term “variant” refers to biologically active forms of the perturbagen sequence (or the polynucleotide sequence that encodes the perturbagen) that differ from the sequence of the initial perturbagen.

The terms “homology” or “homologous” refers to the percentage of residues in a candidate sequence that are identical with the residues in the reference sequence after aligning the two sequences and introducing gaps, if necessary, to achieve the maximum percent of overlap (see, for example, Altschul, S. F. et al. (1990) “Basic local alignment search tool.” J Mol Biol 215(3):403-10; Altschul, S. F. et al. (1997) “Gapped BLAST and PSI-BLAST: a new generation of protein database search programs.” Nucleic Acids Res 25(17):3389-402). It is understood that homologous sequences can accommodate insertions, deletions and substitutions in the nucleotide sequence. Thus, linear sequences of nucleotides can be essentially identical even if some of the nucleotide residues do not precisely correspond or align. The reference sequence may be a subset of a larger sequence, such as a portion of a gene or flanking sequence, or a repetitive portion of a chromosome.

The term “scaffold” refers to a proteinaceous or RNA sequence to which the perturbagen or perturbagen encoding sequence is covalently linked to provide e.g. conformational stability and/or protection from degradation.

The term “endogenous cellular protein” defines a protein, polypeptide or aggregate of polypeptide subunits that are encoded by the native genetic material resident in the selected host cell.

### DETAILED DESCRIPTION OF THE PREFERRED EMBODIMENTS

A. Overview of the Invention.

The invention provides a rapid, efficient way of screening for (i) lethal agents or substances that cause or accelerate death of a cell, or (ii) agents that trigger growth and/or reproductive arrest in a population of cells and, thus, eventually lead to the demise of that population. Both types of agents are referred to herein as “cytotoxic agents,” or “cytotoxic perturbagens” as the end result is the loss of a cell population.

The invention accomplishes this end of efficient screening by providing negative selection assays that first either directly or indirectly select for a lethal phenotype, and then yield direct recovery of the sequence encoding the cytotoxic agent and the modulators of endogenous proteins that create that phenotype. The lethal phenotype may be the result of any number of physiological events resulting in cell death. As non-limiting examples, the cells may die by an active, pre-programmed pathway such as apoptosis or by a more passive, degenerative means such as necrosis, i.e., as a direct result of creating lethality in individual cells. In other instances, the cells may degrade as an indirect result, e.g., via some form of growth arrest. Such growth arrest may be caused by a variety of mechanisms that block normal cellular development, thereby freezing the cell in a given stage of its cell growth cycle (i.e. cell cycle arrest). For example, p16-induced growth arrest halts the cells in the G1 phase of the cell cycle. Furthermore the agents themselves may act by a variety of means. For instance, in the case where the agent is a proteinaceous molecule, the perturbagen may interrupt a target's function by preventing the target from participating in a critical interaction with a second, unrelated molecule in the cell. Alternatively, the target might be an enzyme and the agent may induce a lethal phenotype by binding to the active site and preventing the substrate from interacting with the target molecule. The agent may act by these means or others to induce the desired phenotype.

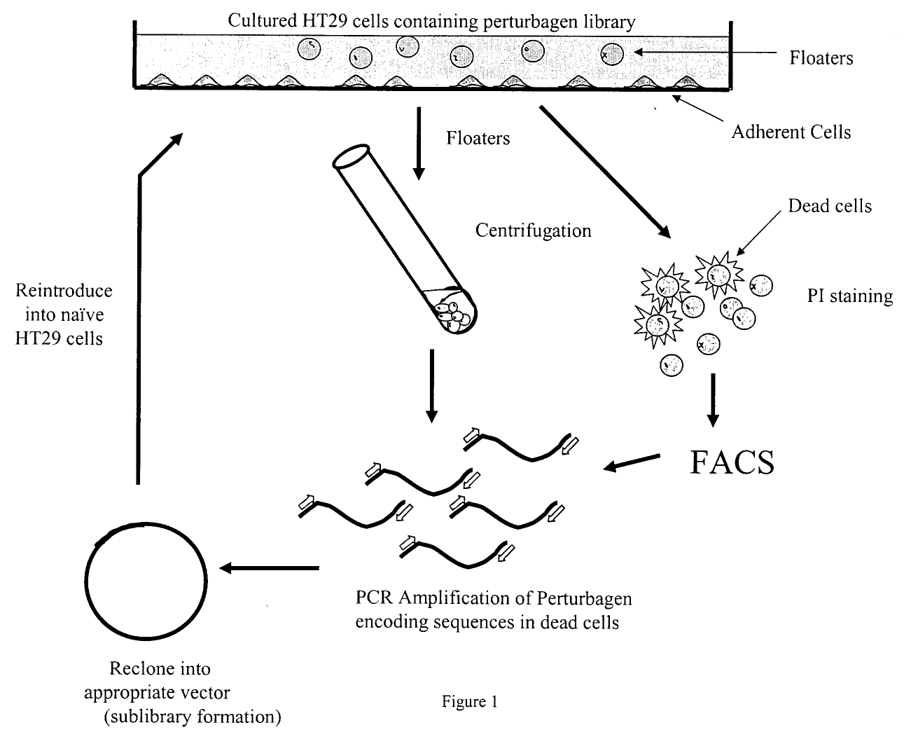

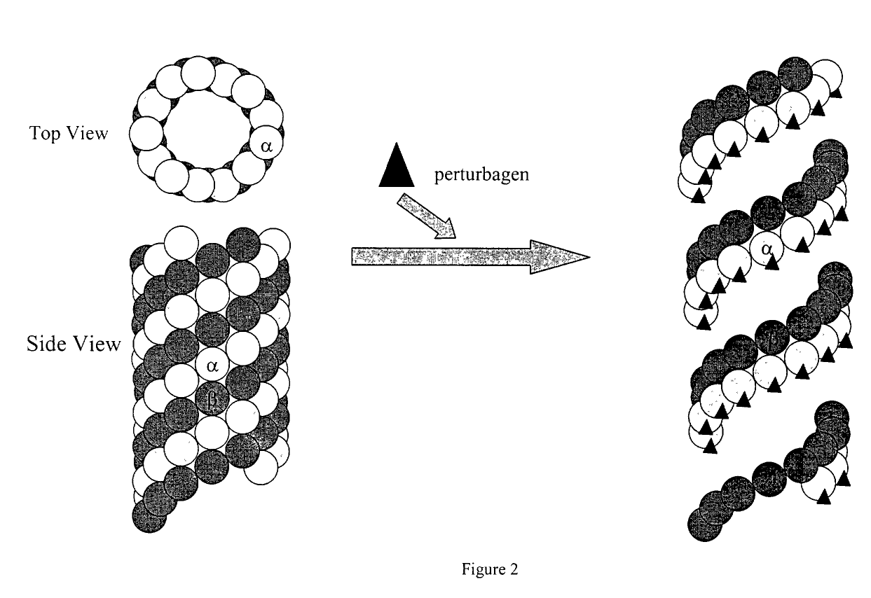

In one aspect, the invention provides a floater assay which utilizes the phenotype of floating or release from the solid support surface to which an adherent cell line is normally attached as a surrogate for cell death. The floater assay utilizes this trait to identify agents that induce a lethal phenotype. To accomplish this, a population of polynucleotide sequences (a “library”) is generated using a variety of techniques familiar to the art. After ligating this material into a standard expression vector, the library is transferred into a population of cells, preferably a population of cells that embody the traits of the disease under study. Subsequently, the culture is screened for sequences that induce cells to lose their adhesion properties (see FIG. 1). For the floater assay to be viable, the cells chosen for study must exhibit a strong adherence to a solid support (e.g. plastic, agarose). Moreover there must be a strong correlation between loss of adhesion (i.e. floating) and cell death. As shown below, such a correlation exists between several lines studied. Thus, by collecting cells that are released from the surface, the assay advantageously identifies one or more relevant sequences from the library that induces the desired phenotype, cell death. Dead and/or dying cells can be separated from the rest of the population by a variety of procedures including but not limited to i) collecting the media that overlays the adherent cell monolayer, or ii) staining the entire population of cells (floating+adherents) with one of several dyes/markers that distinguishes dead from vital cells (e.g. propidium iodide, Apo2.7 antibody, annexin) and separating the desired population by Fluorescent Activated Cell Sorting (FACS). As yet another alternative, dead cells can be collected using the Forward Scatter/Side Scatter option of the FACS analysis that separates cells on the basis of size and granularity. Previous studies have shown that in cultures containing both live and dead cells, two populations (referred to as Pop1 and Pop2) are easily distinguished. Cells that are large and (in general) lightly granulated have been found to be healthy (i.e. alive) and fall into the Pop1 population. In contrast, apoptotic cells are typically smaller and highly granulated, and fall into the Pop2 population. Regardless of whether the dead cells are stained or distinguished by their size/refractive properties, FACS machines are both highly sensitive and efficient (obtaining screening speeds of approximately 10,000 to approximately 65,000 cells or more per minute) thus facilitating identification of biologically relevant sequences that exist at low frequencies within a cell population. Subsequent PCR amplification, sublibrary formation, and re-screening of the sequences encoding cytotoxic agents are derived from the dead cell population enables further enrichment of sequences that induce cell death.

In another aspect, cytotoxic or cytostatic agents can be identified by performing an additional negative selection called a cell-lethal assay. Briefly, a genetic library is introduced into a cell type of choice and cultured over the course of a defined period of time. At the end of this period, dead and/or dying cells are identified using one of several techniques including, but not limited to, staining with i) fluorescently labeled annexin, ii) fluorescently labeled Apo2.7, iii) propidium iodide, or iv) Sytox. Comparison of levels of fluorescence induced by a prospective perturbagen vs. relevant controls enables the identification of important cytotoxic agents. These procedures can be performed manually or in a high-throughput, roboticized format referred to herein as Somata.

The invention is well suited for evaluating the activity of a cytotoxic agent in the presence or absence of other agents (e.g., sensitizers or synergistic reagents). Thus, in some embodiments, the invention may be utilized to evaluate “conditional cytotoxicity,” in which one identifies a potentiating agent (e.g., a sensitizer encoded by a genetic library insert) that increases the sensitivity of a cell to a secondary reagent (e.g., a known chemotherapeutic drug or radiation from a variety of sources, including ultraviolet, X-ray and neutron). Thus the potentiating agent enhances the cytotoxicity of the secondary reagent, rendering a normally subtoxic dosage or exposure of that secondary reagent, cytotoxic. This approach is of particular interest in evaluating candidate agents for ameliorating multidrug resistance (MDR) in, e.g., cancer cells, thereby making such cells susceptible to standard chemotherapeutic agents.

In still other embodiments, the target cells may be pre-sensitized via some agent that does not itself exert a deleterious effect, for example, by addition of a growth factor. In other instances, the target cells may be pre-sensitized by activating the expression of a gene of interest, for example, an oncogene.

The invention also lends itself to readily identifying agents that act in a “cell-specific” manner. This aspect can be accomplished by conducting a counterscreening step utilizing a second cell type. In such embodiments, the invention may be utilized to identify cytotoxic agents that exert a differential cytotoxic effect, for example by selectively killing a first type of cell, while under similar conditions not exerting a cytotoxic effect on a second cell type. As one specific but non-limiting example, a library encoding putative cytotoxic substances may be screened in a first cell population—e.g., a cancerous cell line such as WM35. Agents that cause a lethal phenotype in those cells are then isolated and screened in a second, corresponding primary cell line. Agents that do not cause a lethal phenotype in the non-cancerous cell line are then isolated and further characterized.

B. Identification of a Lethal Phenotype

A variety of methods exist for identifying cells having a lethal phenotype. For example, many methods familiar to those of ordinary skill in the art target cellular components such as surface antigens that arise only upon a cell's entry into an apoptotic or necrotic pathway. In other instances, a change in cellular morphology, physiology, or cellular permeability that characterizes the lethal phenotype may be utilized to identify cells that are dead and/or dying. Such changes include changes in nuclear membrane integrity, “blebbing” of the cell membrane, activation of cellular caspases, contraction of the cell nucleus, and other alterations.

A variety of dyes, fluorescent substrates, stains and antibodies can be used to detect dead and/or dying cells. These materials include without limitation the antibody Apo 2.7, propidium iodide, Sytox dyes, and fluorescent caspase substrates. When an identification agent is fluorescent, cells displaying a lethal phenotype may readily be isolated from cells using a fluorescence activated cell sorter (FACS), a 96 well plate reader or a CCD camera set to detect fluorescent emissions of the proper wave length. In Apo 2.7 assays a fluorescently labeled (e.g. phycoerythrin) Apo 2.7 antibody (Clontech) that recognizes a 38 kD mitochondrial protein that is only exposed under conditions of apoptosis is used to determine the fraction of the cell population that is dead and/or dying. Alternatively, a fluorescently labeled annexin molecule can be used to identify and isolate the dead and/or dying cells within a population. Annexin V binds to a lipid moiety, phosphatidylserine, which is normally located on the inner leaflet of the plasma membrane. When many cells undergo apoptosis (programmed cell death) this lipid translocates to the outer leaflet of the plasma membrane thus enabling exogenously added, fluorescently-coupled, Annexin V to interact with the lipid and thereby labeling the cell that is undergoing apoptosis. In addition, during the normal course of necrosis (non-apoptotic cell death), when the integrity of the plasma membrane becomes compromised, fluorescently-conjugated Annexin V is able to enter the cell and interact with the lipid, again serving to tag a dead cell. Another representative fluorescent entity is SytoX™ (Molecular Probes), which is a membrane-impermeable dye that is able to enter cells during the latter stages of either apoptosis or necrosis when the plasma membrane becomes compromised. Upon entry into the cell, Sytox interacts with the cell's DNA and fluoresces, thereby allowing detection of dead cells.

In contrast to techniques that utilize Annexin V, Sytox, or Apo2.7, caspase based technologies can be utilized to detect the activation of one or more caspases (proteases) that are triggered during apoptosis. One commercially available kit (e.g. the FLICE fluorescent kits, Clontech) detects the shifts in fluorescence emission of 7-amino-4-trifluoromethyl coumarin (AFC) that is conjugated to a tetrapeptide (e.g. IETD) recognized by the FLICE caspase. Under normal, non-apoptotic conditions, the AFC-tetrapeptide conjugate is stable and emits blue light (Lambda max=400 nm). In contrast, when the FLICE caspase is activated during apoptosis, the substrate molecule is proteolytically cleaved, producing a byproduct that emits a green fluorescence at 505 nm.

In still other instances, a gross morphological characteristic that is readily detected may be used as a surrogate for the lethal phenotype. As discussed previously, dead and/or dying cells exhibit changes in both size and granularity and these properties can be utilized to separate e.g. apoptotic cells from healthy cells contained in a culture. Thus under certain conditions, assays that utilized FACS machines or related tools that detect light scattering properties of samples can be used to identify cells undergoing cell death.

C. Target Cells

A wide variety of different cell types derived from both plant and animal sources are suitable for use as target cells. Host cell lines for use in the methodology described herein typically embody such desirable traits as i) short cell cycle (i.e. 20-36 hr. doubling time), ii) amenability to high throughput procedures (e.g. FACS) without undue loss of membrane integrity or viability, iii) susceptibility to standard techniques designed to introduce various forms of foreign DNA and iv) carrying some relation to a known pathology or disease state. In addition, in order to use floater populations as a method of identifying and enriching for dead and/or dying cells in a negative selections, cell lines preferably display two additional features: i) in a stable untreated cell population, the greater majority of cells are adherent to the solid support (e.g. plastic, gelatin) and the background rate of floater cells (i.e. the rate at which an untreated population exhibits floaters) is relatively low (<1%) and ii) in an untreated or treated cell population (i.e. one exposed to putative cytotoxic agents and optionally, secondary agents), a high percentage of the floater cells correlate with the dead and/or dying cell population.

One non-limiting example of a satisfactory and acceptable cell line is the mammalian colorectal adenocarcinoma cell line, HT29. HT29 (ATCC# HTB-38) cells divide rapidly, are highly susceptible to retroviral infection and other methods of introducing foreign genetic materials and can express/maintain said materials for long periods of time using a variety of selectable markers common to the field (e.g. neomycin, puromycin). In addition, previous studies have shown that HT29 cultures transduced with control retroviral vectors exhibit low levels of floaters (<1%) and that a sizeable percentage of these cells (˜40%) can be shown to be dead and/or dying (see U.S. Ser. No. 09/504,132). Thus, HT29 serves as an acceptable cell line for isolating performing negative selection procedures.

In addition, it is understood that there are many other suitable host cell lines may be used in these studies. The breadth of the invention is not limited by the type of host cell employed and cultures derived from epithelial cells, endothelial cells, stem cells, mesenchymal cells, fibroblasts, neuronal cells, hematopoietic cells, and others can be used. Thus cell lines including but are not limited to i) SW620 colon cancer cells (colorectal adenocarcinoma, ATCC # CCL-227), ii) the metastatic mammary epithelial cell tumor T-47D (ATCC #HTB-133), HCT15 (a colorectal adenocarcinoma, ATCC # CCL225), and iv) H1-HeLa cells (human cervical adenocarcinoma cells (ATCC #: CRL-1958). Furthermore the invention can be applied to cells that have been genetically altered so as to have specific properties or traits. One such cell line is 96C that is an endothelial cell that has been transformed and immortalized by the introduction of SV40 Lg T antigen, hTert, and V12H-Ras. Thus, cells that have been immortalized with these and other well-known genes such as HPV-E6, HPV-E7, the Epstein-Barr Virus BARF1 gene, the human T-cell leukemia virus type 1 TAX gene, and others, can be employed. Lastly it should be noted that a number of normal cells including human mammary epithelial cells (HMEC, Clonetics) and human umbilical vein endothelial cells (HuVECs, Clonetics) can be utilized for counter selection screens to assist in the identification of agents that kill diseased cells but have little or no effect on normal cells. Furthermore, it is within the scope of the invention to identify perturbagens that kill normal cell lines. For instance, in cases where a diseased cell depends upon the function of a wildtype cell (e.g. a tumor relying upon the formation of new, wildtype blood vessels i.e. angiogenesis) such normal cells can be targeted using agents developed from screens designed to identify perturbagens that kill or limit the growth of normal cells.

D. Libraries

Libraries used in these procedures are developed from a variety of sources and can consist of proteinaceous or ribonucleotide elements. Proteinaceous libraries are derived from cDNA, gDNA, and random, synthetic oligonucleotides sources and are synthesized using current available methods. In one preferred instance, libraries are cDNA expression libraries constructed from the messenger RNA of one or more specific tissues (e.g. brain, placenta, kidney, liver). cDNA libraries can be constructed from polyA RNA by priming the first strand synthesis off of an oligo dT primer/linker molecule or by randomly synthesizing fragments of expressed mRNA molecules using random primers. (Froussard, P. (1992) “A random-PCR method (rPCR) to construct whole cDNA library from low amounts of RNA” NAR 2(11): 2900). Using the appropriate vector, cDNA libraries can be transformed into a cell, transcribed, and then translated into a peptide sequence that can be as short as 3 amino acids in length and as long as, for instance, 3,000 amino acids in length. Natural proteinaceous libraries can also be prepared from genomic DNA. gDNA libraries can be constructed from a variety of organisms including, but not limited to, bacteria, yeast, and C. elegans. In some instances, these genomic based libraries are constructed by digesting gDNA with one or more restriction enzymes, size fractionating the resultant product, and then inserting the sized material into the vector of choice using complementary restriction sites. In other instances, genomic DNA can be sheared by, for instance, vortexing or sonication, size fractionated, polished to repair fragment ends, and then blunt-end cloned into the vector of choice (Ramer, S. et al. (1992) “Dominant genetics using a yeast genomic library under the control of a strong inducible promoter” PNAS 89: 11589-11593). Like the previously described cDNA libraries, the translated product of the genomic DNA library can vary in length greatly and be as small as 3 amino acids in length, and as long as 3,000 amino acids. In addition, both genomic and cDNA expression libraries can be expressed freely or scaffolded onto a larger moiety for added stability.

Libraries of proteinaceous cytotoxic agents can also be synthetic in nature. For instance, libraries of random, or biased-random polynucleotides sequences of a defined length can be constructed and expressed from one or more expression vectors common to the art. Again, the peptide sequence encoded by the oligonucleotide is expressed as a free, unattached agent. In other instances, the peptide is fused to the N-terminal, C-terminal, or internal sites of a scaffold molecule that increases peptide stability (see, for example, Caponigro et al. (1998) “Transdominant genetic analysis of a growth control pathway.”PNAS 95:7508-7513; Caruthers, M. H. et al. (1980) Nucleic Acids Symposium, Ser. 7:215-223; Horn, T. et al. (1980) Nucleic Acids Symposium, Ser. 7:225-232; Cwirla, S. E. et al. (1990) “Peptides on phage: a vast library of peptides for identifying ligands.” Proc Natl Acad Sci 87(16): 6378-82; Abedi, M. R. (1998) “Green Fluorescent Protein as a Scaffold for Intracellular Presentation of Peptides.” NAR 26: 623-630).

In other embodiments, the library is composed of RNA molecules that are themselves active (i.e. the molecule is not acting through the correlative encoded protein or peptide that results from translation of the RNA). In some cases such libraries are constructed intentionally (i.e. constructed without translation initiation sequences). In other instances, the library is a synthetic oligo, cDNA, or gDNA library, the contents of which are intended to be translated, yet fortuitously, one or more members of the library are active as RNA molecules.

As was the case with proteinaceous perturbagens, RNA derived perturbagens can be fused to RNA-based scaffold sequences at 5′, 3′ or internal sites. The fusion of library sequences to a second entity can increase the relative effectiveness of a potential cytotoxic agent by increasing the stability of either the messenger RNA.

In some instances, scaffolds may be a relatively inert protein, (i.e. having no enzymatic activity or fluorescent properties) such as hemagglutinin. Such proteins can be stably expressed in a wide variety of cell types without disrupting the normal physiological functions of the cell. In other instances, scaffolds may serve a dual function, e.g., increasing perturbagen stability while at the same time, serving as an indicator or gauge of the level of perturbagen expression. In this case, the scaffold may be an autofluorescent molecule such as a Green Fluorescent Protein (Clontech) or embody an enzymatic activity capable of altering a substrate in such a way that it can be detected by eye or instrumentation (e.g. β galactosidase). For example, in the invention described herein, various molecular techniques that are common to the field are used to link the perturbagen library to, e.g., the C-terminus of a nonfluorescent variant of GFP. “dEGFP” (also referred to as “dead-GFP”) is a such nonfluorescent variant brought about by conversion of Tyr→Phe at codon 66 of EGFP (Clontech). By linking the perturbagen library to this molecule, each library member is fused to a separate dEGFP molecule. Such chimeric fusions can easily be detected by Western Blot analysis using antibodies directed against GFP and are useful in determination of intracellular expression levels of perturbagens. In addition, the perturbagen sequences or the scaffold to which they are attached can be modified with various localization signals so that the perturbagen may be directed to a particular compartment within the host cell. For example, proteinaceous agents can be directed to the nucleus of certain cell types by attachment of a nuclear localization sequence (NLS); a heterogeneous sequence made up of short stretches of basic amino acid residues recognized by importins alpha and/or beta.

Libraries may be obtained from commercial sources, or may be constructed. Furthermore, the libraries used in HTS procedures may be sublibraries that result from previous, successive, “batch” screens designed to enrich for clones embodying the desired phenotype. Alternatively, because some clones may be required in multiple copies in order to see or observe a weak phenotype, library (batch) cycling may be forsaken and HTS procedures may be performed on a nascent library.

E. Expression Vectors

The DNA sequence encoding each cytotoxic agent (or variant or fragment thereof) may be inserted into an expression vector that contains the necessary elements for transcriptional/translational control in a selected host cell. Thus the DNA sequence may be expressed for e.g., testing or screening in a bioassay such as those described herein, or for production and recovery of the proteinaceous agent once the agent has been identified. Methods that are well known to those skilled in the art are used to construct expression vectors containing sequences encoding the cytotoxic agents and the appropriate transcriptional and translational control elements. These methods include in vitro recombinant DNA techniques, synthetic techniques, and in vivo genetic recombination (see Sambrook, J. et al. (1989) “Molecular Cloning, A Laboratory Manual”, Cold Spring Harbor Press, Plainview N.Y.; Ausubel, F. M. (1993) “Current Protocols in Molecular Biology”, Wiley, John & Sons, Incorporated).

The vector may include regulatory sequences, such as enhancers, constitutive and inducible promoters, and 5′ and 3′ untranslated regions, mRNA stabilizing sequences or scaffolds, for optimal expression of the perturbagen in a given host. For instance, intracellular perturbagen levels can be modulated using alternative promoter sequences such as CMV, RSV, and SV40 promoters, to drive transcription (see, for example, Zarrin, A. A. et al. (1999) “Comparison of CMV, RSV, SV40 viral and V lambdal cellular promoters in B and T lymphoid and non-lymphoid cell lines.” Biochim Biophys Acta. 1446(1-2):135-9). Alternatively, inducible promoter systems (e.g. ponesterone-induced promoter, PIND, Invitrogen, see Dunlop, J. et al. (1999) “Steroid hormone-inducible expression of the GLT-1 subtype of high-affinity 1-glutamate transporter in human embryonic kidney cells.” Biochem Biophys Res Commun. 265(1): 101-5), tissue specific enhancers (see, Latham, J. P. et al. (2000) “Prostate-specific antigen promoter/enhancer driven gene therapy for prostate cancer: construction and testing of a tissue-specific adenovirus vector.” Cancer Research 60(2): 334-41), or scaffolding molecules (see, for example, see Abedi, M. et al. (1998), “Green fluorescent protein as a scaffold for intracellular presentation of peptides.” Nucleic Acid Research 26(2): 623-630) can be used to modulate intracellular perturbagen levels.

Specific initiation signals may be used to achieve more efficient translation of sequences encoding the agent. Such signals include the ATG initiation codon and adjacent sequences, e.g. the Kozak sequence. In cases where sequences encoding the agent and its initiation codon and upstream regulatory sequences are inserted into the appropriate expression vector, no additional transcriptional or translational control signals may be needed. However, in cases where only coding sequence is inserted, exogenous translational control signals including an in-frame ATG initiation codon are provided by the vector. Such exogenous translational elements and initiation codons may be of various origins, both natural and synthetic.

In some instances, sequences that stabilize the RNA transcript or direct the RNA sequence or its encoded peptide to a particular compartment may be included (see, for instance, Wood Chuck post transcriptional regulatory element, WPRE, Zufferey, R. et al. (1999) “Woodchuck hepatitis virus posttranscriptional regulatory element enhances expression of transgenes delivered by retroviral vectors.” J Virol 73(4): 2886-92).

A variety of paired expression vector/host systems may be utilized to contain and express sequences encoding the cytotoxic agent. As one of ordinary skill will appreciate, the selection of a given system is dictated by the purpose of expression: e.g., bioassay. These include, but are not limited to, insect cell systems infected with viral expression vectors (e.g. baculovirus), plant cell systems transformed with viral expression vectors (e.g. tobacco mosaic virus, MTV) or with bacterial expression vectors (e.g. Ti or pBR322 plasmids) and mammalian cell systems (e.g. COS, CHO, BHK, 293, or 3T3 cells) that use episomal, adenoviral, or retroviral expression systems. The host cell employed does not limit the invention.

Plant systems may be used for expression and identification of cytotoxic agents. Transcription of sequences encoding perturbagens may be driven by viral promoters, e.g. the 35S and 19S promoters of CaMV used alone or in combination with the omega leader sequence from TMV (Takamatsu, N. (1991) “Deletion analysis of the 5′ untranslated leader sequence of tobacco mosaic virus RNA.” J Virology 65:1619-22). Alternatively, plant promoters such as that of the small subunit of RUBISCO or heat shock promoters may be used. (see, for example, Coruzzi, G. et al. (1984) “Tissue-specific and light-regulated expression of a pea nuclear gene encoding the small subunit of ribulose-1,5-bisphosphate.”EMBO J. 3:1671-80; Broglie, R. et al. (1984) “Light-regulated expression of a pea ribulose-1,5-bisphosphate carboxylase small subunit gene in transformed plant cells.” Science 24:838-843).

In an insect system, Autographa californica nuclear polyhedrosis virus (AcNPV) is used as a vector to express foreign genes in Spodoptera frugiperda cells. The gene of interest may be cloned into non-essential regions of the viral genome and placed under control of, for instance, the AcNPV promoter. Successful insertion of gene coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith, et al., (1983) “Molecular Engineering of the Autographa californica Nuclear Polyhedrosis Virus Genome: Deletion Mutations Within the Polyhedrin Gene,” J. Virol., 46:584-593; U.S. Pat. No. 4,745,051).

For production of recombinant proteins in mammalian systems, transient or stable expression of proteinaceous agents in cell lines may be used. For transient expression vectors such as EBV-based vectors can be used (Teixeira La., et al. (2001) “An efficient gene transfer system for hematopoietic cell line using transient and stable vectors.” J Biotechnol 15;88(2):159-65). Such plasmids can encode elements necessary for the expression of the perturbagen as well as selectable markers for maintenance. Alternatively, cells can be transfected using, for instance, retroviral, adenoviral, or adeno-associated viral agents as delivery systems for the perturbagen. These delivery systems splice the desired cDNA, gDNA, or synthetic peptide fragment expression sequence into the host genome, resulting in stable expression of the perturbagen. For example, retroviral vectors (e.g. LRCX, Clontech) may be used to introduce and express cytotoxic agents in a variety of mammalian cell cultures. Such vectors may rely on the virus' own 5′ LTR as a means of driving expression or may utilize alternative promoters/enhancers (e.g. those of CMV, RSV and SV40, PIND, TAT/HIV2) to regulate the cytogen's expression levels.

In each case described above, the selected construct can be introduced into the selected host cell by direct DNA transformation or pathogen-mediated transfection. The terms “transformation”, “transduction” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Preferred technologies for introducing cytotoxic agents into mammalian cells include, but are not limited to, retroviral infection as well as transformation by EBV or similar episomally-maintained viral vectors (Makrides, S.C. (1999) “Components of vectors for gene transfer and expression in mammalian cells.”Protein Expr Purif 17(2): 183-202). Other suitable methods for transforming or transfecting host cells can be found in Maniatis, T. et al (“Molecular Cloning: A Laboratory Manual.”Cold Spring Harbor Laboratory Press) and other standard laboratory manuals.

In some instances, a preliminary selection is performed to verify and select for host cells have been successfully transformed/transfected. To accomplish this the vector is introduced into the host cell of choice and then switched to selective media. The selectable marker confers resistance to a selective agent, and thus, only those cells that successfully express the introduced sequences survive in the selective media. Any number of selection systems may be used to recover transformed/transfected cell lines. These include, but are not limited to, the herpes simplex virus thymidine kinase and adenine phosphoribosyltransferase genes, for use in tk- or apr-cells, respectively (see e.g. Wigler, M. et al. (1977) “Transfer of purified herpes virus thymidine kinase gene to cultured mouse cells.” Cell 11: 223-32; Lowy, I. et al. (1980) “Isolation of transforming DNA: cloning the hamster aprt gene.” Cell 22: 817-23). Also antimetabolite, antibiotic, or herbicide resistance can be used as the basis for selection. For example, dhfr confers resistance to methotrexate; neo confers resistance to the aminoglycosides, neomycin and G-418, and als and pat confer resistance to chlorsulfuron and phosphinotricin acetyltransferase, respectively. (see Wigler, M. et al. (1980) “Transformation of mammalian cells with an amplifiable dominant-acting gene.” PNAS 77:3567-70; Colbere-Garapin, F. et al (1981) “A new dominant hybrid selective marker for higher eukaryotic cells.” J. Mol. Biol. 150:1-14). Additional selectable genes have been described, e.g. trpB and hisD, which alter cellular requirements for metabolites. Visible markers, e.g. anthocyanins, green, red or blue fluorescent proteins (Clontech), B glucuronidase and its substrate B glucuronide, or luciferase and its substrate luciferin, may also be used. Although such markers are not selectable in the classical definition of the word, cells expressing such markers can be easily separated from the rest of the population using FACS and related procedures. Resistant clones containing stably transformed cells may be propagated using tissue culture techniques appropriate to the cell type.

F. Floater Cell Assays and Enrichment

In some embodiments of the invention, the lethal phenotype (e.g., apoptosis or necrosis) is selected for by using a negative selection assay that uses as a surrogate the selection property of disattachment from a culturing surface—referred to herein as a “floater assay.” Accordingly, a cell population that is enriched or even highly enriched in cells displaying the lethal phenotype may be collected simply by collecting the cells that “float” in the culture media after exposure to a cytotoxic agent.

Such a floater assay embodiment first involves the selection of a suitable target cell type, for example a cell type that correlates to a tissue or disease state of interest and which displays a suitably low background of spontaneous disattachment (i.e., disattachment of non-apoptotic cells), and high correlation between disattachment and a lethal phenotype following exposure to a lethality-inducing dose of a cytotoxic agent. Suitable cell types can be selected or identified as follows. First, a cell type relating to the disease of interest is selected. Next, a cell culture is established using standard techniques. Then the cell culture is separated into two populations—“floaters” and adherent cells. The “floater” population is recovered by withdrawing the culture medium from the culture plate or flask, and then culling the cells from that medium by e.g. centrifugation. The adherent population is obtained by trypsinizing the cells that remained adhered to the culture support following withdrawal of the culture medium. Each population is then counted, and the relative number of spontaneous floaters to adherent cells is calculated. The floater cell and adherent cell populations are then analyzed to determine the number of apoptotic or necrotic cells in each. Preferred cell types provide a high correlation between the lack of adherence and the lethal phenotype—i.e., the floater population is relatively heavily populated with dead or dying cells, while the adherent population is relatively heavily populated with viable cells.

Floater cells may present differing concentrations of lethal and non-lethal phenotypes, depending on the cell type from which they are derived. For example, in some cell lines, viable cells (i.e., healthy, living cells that are still undergoing cellular division and/or which are not replicating but which still display normal cellular metabolism and physiology) are not adherent. In such cell lines, the surrogate phenotype of non-adhesion does not correlate to apoptosis, and thus such cell lines are not suitable for the floater assay technique described herein (but may be used for negative selections using direct recovery of genetic material from dead or dying cells culled by, e.g., FACS analysis). In still other cell lines in which viable cells normally adhere to a plating surface, a significant percentage of the viable, non-apoptotic cells may enter the floater cell populace. Again, such cell lines are unsuitable for floater-type assays. In other cell lines, some significant proportion of the floater cell populace may be non-apoptotic, non-viable cells—for example, cells that have died from cellular processes other than apoptosis. Cell lines providing such mixed “floater” populations can be utilized the assay techniques described herein if suitable controls are employed and/or a second, independent identification method (e.g., Apo 2.7 or Propidium Iodide) is utilized in conjunction with the floater assay technique.

After identifying a suitable cell line, the chosen cell type is then exposed to the putative cytotoxic agents. If the cytotoxic agent is or is encoded by nucleic acid, then this is readily accomplished by providing a population of the selected cell type with a library encoding a variety of such agents, for example by following standard procedures for the construction and transduction of retroviral libraries. The treated target cells are then cultured for sufficient time to ensure establishment of the lethal phenotype, following which the “floaters” or disattached cells in the cell culture medium are collected and processed to extract the genetic material. The DNA from the dead or dying cells is then retrieved and amplified, and at least partially sequenced in order to identify what cytotoxic agent(s) correlate to the apoptotic lethal phenotype. Alternatively the population of sequences can be reintroduced into a naive population of host cells and recycled through the procedures for further enrichment of sequences that induce a cytotoxic phenotype. As yet another alternative, the agents may be cloned and introduced into an alternative assay (e.g. the cell-lethal assay) to determine if the clone embodies desirable cytotoxic or cytostatic properties.

Alternatively, the floater cell population as a whole can be utilized as a subpopulation that has been enriched for one or more lethal phenotypes. When one wishes to distinguish, e.g., apoptotic cells from necrotic cells, then the above strategy may be used to enrich a given target cell subpopulation for lethal phenotypes. The enriched subpopulation may then be further segregated using, e.g., an apoptosis-specific identification strategy (e.g., Apo 2.7) and FACS sorting to obtain a purified apoptotic cell fraction.

G. Cell Lethal Assays and Somata

Due to the background inherent in all assays, the population of sequences derived from floater assays contain both cytotoxic and non-cytotoxic sequences. To identify the relevant cytotoxic elements within this sublibrary, a second negative selection referred to as a cell lethal assay is performed. Briefly, individual sequences are transduced into a population of host cells and cultured over the course of a defined period of time. Subsequently, the cells are stained with one or more reagents capable of distinguishing between live and dead (and/or dying) cells and comparisons are made with the appropriate controls to identify cytotoxic agents. Cultures that (in comparison with relevant controls) have higher numbers of dead cells can be considered to contain a cytotoxic agent. While one preferred application of the technology is to identify clones that induce cell lethality, the application can be used to identify agents that have cytostatic properties. This can be accomplished by i) introducing the library into a population of host cells, ii) culturing the sample for an appropriate period of time, and iii) treating the sample with a mild detergent (e.g. saponin, NP40) that permeabilizes the live cells present in the population. Subsequent staining of the cells with any of the reagents described previously will enable one to determine the total cell number in a given culture and comparison of these numbers with the appropriate controls enables one to identify cytostatic agents.

In one embodiment, clones that are identified to be toxic in the primary cell line can be introduced into secondary cell lines (both diseased and normal) to determine the specificity of the agent involved. For instance, agents that are identified to be toxic in HT29 colon cancer cells can be introduced into SW620 cells, HeLa cells, HEK293 cells, as wells as two normal cell cultures, HuVECs and HMECs, to determine the breadth and specificity of the perturbagen action. Desirable agents have strong cytotoxic and/or cytostatic effect on diseased tissues but have little or no effect in normal cells.

The procedures described in the cell lethal assay are highly adaptable to automation. One preferred form of automation, referred to as Somata, has been adapted to the negative selection procedures, enabling the screening of tens of thousands of individual clones. In one non-limiting example of how the automated negative selection assay is performed, individual plasmid clones are grown in bacteria cells on selective agar plates. These clones are subsequently picked, transferred to liquid culture in a 96 or 384 well format, and processed for plasmid purification. Robotics are then used to transfer the appropriate amount of the purified plasmid (along with an envelope plasmid) to a second plate that contains 293pg packaging cells. Together, the two plasmids are transfected into the packaging cell line and viral supernatants are recovered and used to transduce a third plate containing the host assay cell line. Infected host cells are cultured for a defined period, stained with e.g. Sytox Green, and analyzed to compare the levels of cell death induced by each clone with the appropriate controls.

The steps involved in Somata (i) DNA preparation, ii) retroviral packaging, iii) transduction, and iv) bioassay) are linear, co-dependent, and modular. As successful completion of each step is necessary for attainment of an accurate screen, certain parameters must be optimized at each stage of the procedure. For instance, in DNA preparation, the product of the procedures must be of sufficient quantity and purity to allow viral packaging to take place. Similarly, packaging of retroviral vectors must yield viral supernatants that are of sufficient purity and titer to enable a high percentage of the test cell line to be transduced. Due to the cumbersome nature of manually performing large numbers of bioassays and the need to achieve clear and reproducible results, a high throughput format using modem automations has been adopted. For instance, individual plasmid colonies are isolated from selective agar plates using robotic “colony pickers” (see, Uber, D. C. et al. (1991) “Application of robotics and image processing to automated colony picking and arraying.”Biotechniques, 11(5) 642-647; Jones, P. et al. (1992) “Integration of image analysis and robotics into a fully automated colony picking and plate handling system.” Nucleic Acids Res. 20(17): 4599-606.). To isolate each perturbagen-encoding retroviral vector, mini-bacterial cultures grown in 96- or 384-well plates are processed using standard anion-exchange technologies that have been adapted for high-throughput procedures. (see QIAwell Ultra BioRobot Kits and Bio-Robot 8000, Qiagen®, or Millipore Multiscreen automated with the Beckman FX). Similarly, highly infective retroviral supernatants are prepared by introducing each plasmid (along with the appropriate co-vectors, e.g. VSV-G envelope expression plasmid) into 293gp packaging cells similarly plated in either the 96-or 384 well format. Like the previously described plasmid purification procedures, transfection of the 293gp cells with retroviral DNA and collection of the resultant viral supernatants are accomplished using common, “off-the-shelf” automated technologies. In one non-limiting example, automation is configured with a microtiter plate hotel/incubator (Cytomat 6000, Kendro Laboratory Products, Newtown, Conn.), a 96-channel pipettor (Multimek™ 96, Beckman Instruments, Inc., Fullerton, Calif.), a microplate hotel (Platestak™, CCS Packard, Torrance, Calif.), and a plate handler (Pick and Place, Fiore Automation, Salt Lake City, Utah). Bacterial colony picking is performed with an AutoGenesys (Autogen, Framingham, Mass.). Custom ActiveX™ servers from Fiore Automation controls each robotic instrument. These servers are integrated by an instrument management software package, Capitano (Fiore Automation) that allows the end user to create, save, and execute sophisticated process control protocols.

The libraries used in HTS procedures may be sublibraries that result from previous, successive, “batch” screens (i.e. floater assays) designed to enrich for clones embodying the desired phenotype. Alternatively, because some clones may be required in multiple copies in order to see or observe a moderate or weak phenotype, library (batch) cycling may be forsaken and HTS procedures may be performed on a nascent library (e.g. an unenriched cDNA, genomic, or peptide library).

The Somata aspect of the invention is not confined to cell lethal assays and can be applied to detect other cellular phenotypes using TransFACS, or viral pathogen screening procedures (see U.S. Pat. No. 5,955,275, and U.S. Ser. No. 09/259,155, incorporated by reference herein). In the automated pathogen assay, libraries of agents are tested for the ability to protect host cells from pathogen infections and/or pathogen induced cell death. In one non-limiting example, the automated “Viral Assay”, expression libraries are tested to identify individual members that are capable of protecting a given host cell from cell death induced by a particular viral pathogen. In the example given, agents that are capable of limiting infection of rhinovirus (RV14) in H1-HeLa cells are described. H1-HeLa cells are transduced with a cDNA expression library, cultured for a brief period of time, and then challenged with a quantity of RV14 that his sufficient to induce 100% cell death. Subsequently, wells that are found to contain live cells are judged to harbor a perturbagen that blocks some aspect of the viral replication pathway. In the automated Trans-FACS assay, molecules that induce changes in the transcriptional state of, for instance, a unique reporter, are identified. In the non-limiting example given, multiple agents capable of altering the transcription state of a reporter construct that consists of tandem TBE sequences (TCF-4 binding elements) operably linked to a reporter gene, Green Fluorescent Protein (GFP) are described.

A variety of methodologies can be used to detect agents that induce the desired phenotype. In high-throughput TransFACS assays, cells may be detached from the 96- or 384-well plate using, for instance, trypsin, and analyzed by FACS to determine the fraction of cells expressing the desired phenotype (e.g. a desired level of fluorescence). Alternatively, a signal, such as fluorescence emission, can be measured from each well using a CCD camera, or fluorescent plate reader. For instance, in Schroeder et al., (Schroeder and Neagle, J. Biomol. Scr. 1:75-80, 1996) the authors used low angle laser scanning illumination and a mask to selectively excite fluorescence within approximately 200 microns of the bottom of 96 well plates. This procedure reduces the background when imaging cell monolayers and provides a signal that represents an overall average of the population. In viral and negative selection assays, the population contained in each well can be stained with, for instance, propidium iodide, Sytox (Molecular Probes), or equivalent agents that distinguish between living and dead cells. Subsequent to these staining procedures, samples can be illuminated with the proper wavelength of light and (in combination with various permeablization procedures) analyzed to determine the total cell number, or, for instance, the ratio of living cells to dead cells.

H. Recovery of Genetic Material

A variety of methods can be used to recover the DNA encoding the cytotoxic agents from the host cell. One preferred method of recovery uses a specific set of oligonucleotide primers and PCR as a method of recovery. Once the cells bearing the putative cytotoxic agents have been screened and those cells having a lethal phenotype either identified, directly via a staining technique or indirectly via a surrogate phenotype such as lack of adhesion, the genetic material is amplified from the dead and/or dying cells. Briefly, genomic DNA is prepared from the dead and/or dying cells and this material is used as a template for PCR amplification. PCR primers containing homology to the vector are selected so as to amplify the region encoding the putative cytotoxic agents. The genetic material may then be wholly or partially sequenced using techniques familiar to those of skill in the art, and can be reconstituted as a sublibrary for a second selection or for a counter selection.

I. Assaying for Cell-Specific Cytotoxic Agents

In some embodiments, the methods of the invention may be applied to identify agents that exert a differential cytotoxic effect—i.e., are cytotoxic to one cell population but not to another. Such embodiments are particularly advantageous for identifying agents that will act with specificity against a given diseased cell, while leaving non-diseased cells partially or wholly unaffected. Such applications are particularly advantageous in that the agents so identified are expected to provide therapeutic advantages such as lack of undesirable side effects, lower therapeutic dosages, and the like.

One general approach is as follows. The negative selection strategy for selecting lethal phenotypes (e.g., apoptosis) is implemented as described above, utilizing the cell type against which a cell-specific agent is sought. Upon completion of this step, the genetic material encoding or embodying the cytotoxic agents is isolated, and reintroduced into a second population of cells that is, or is representative of, the cell type for which it is desired that the cytotoxic agent be relatively or completely non-toxic. In this second counter selection step, one of two strategies may be employed. First, the cells exhibiting a lethal phenotype may be collected and the corresponding genetic material be evaluated so as to eliminate putative cell-selective agents (as having been demonstrated to be non-cell specific). Conversely, the counter selection step may employ a positive selection strategy: isolating the genetic material that corresponds to the cells in the second population that do not exhibit the lethal phenotype—i.e., continue to grow in the presence of the cytotoxic agent.

J. Assaying for Conditional Cytotoxicity

The basic negative selection strategy described above may be modified slightly to identify agents that increase sensitivity of a target cell to a known cytotoxic agent (termed herein, “conditional cytotoxicity”). Such embodiments are particularly advantageous for identifying agents that can be used as sensitizers, given in conjunction with the known cytotoxic agent. Such a strategy permits a lower dosage of the known cytotoxic agent to be administered, with correspondingly lower incidence or severity of unwanted side effects.

One general approach is as follows. A target cell type is selected, and a cytotoxic substance of interest (referred to herein as a “secondary reagent”) is selected. Next, a “standard kill curve” (i.e., dose-response curve, wherein increasing amounts of agent are presented to target cells, and the resultant cell death monitored and plotted) is prepared for that cell type and cytotoxic substance. From the standard kill curve, a “subtoxic threshold” dosage of the secondary reagent (i.e., the largest dosage from the kill curve that does not initiate cell death in the target cell population) is selected for further study. A population of the target cells is then provided with one or more putative cytotoxicity-enhancing agents (e.g., in the form of a genetic library), and subsequently exposed to the selected sub toxic threshold dosage of the secondary reagent. A negative selection as described elsewhere herein is then conducted, and transformed target cells that die in response to the subtoxic amount of the secondary reagent are collected and the corresponding cytotoxicity-enhancing agent identified. If that agent is a proteinaceous or nucleic acid biomolecule, then the genetic material that encodes or comprises the agent is isolated and evaluated, for example by the PCR amplification and sequencing strategy described elsewhere herein. Subsequently, each agent is retested in the negative selection in the absence of the secondary reagent to determine if the toxicity is solely due to the perturbagen or a combination of the perturbagen with the sub-toxic levels of the secondary reagent.

K. Preconditioning

In some embodiments of the invention, a preconditioning step may be added to the negative selection strategy. In such embodiments, a population of target cells is first exposed to a preconditioning agent. The cells are then exposed to the putative cytotoxic agents (e.g., a genetic library). Again, the selection collects cells displaying a lethal phenotype (e.g., apoptosis or necrosis), and isolates the corresponding cytotoxic agents, as described elsewhere herein. This step results in the identification of agents that act in the presence of the preconditioning agent.

Optionally, a second selection step (a positive selection) may be used to identify agents that act only in the presence of the preconditioning agent. In such an embodiment, a second population of the target cell is exposed in a similar manner to the cytotoxic agent(s) isolated in the first (negative) selection step, but without the prior step of exposure to the preconditioning agent. Cells that live are collected, and the corresponding cytotoxic agent identified, as described elsewhere herein.

A variety of preconditioning agents will be known to those of skill in the art. Generally, these agents will be involved in metabolic pathways related to cellular growth or death. Non-limiting examples include growth factors such as the activated EGF receptor, activated oncogenes such as ras or myc, knockouts of genes such as p53, p16 or Rb, and the like.

L. Properties of Perturbagens

1. General Properties

The invention encompasses both the phenotypic probes (perturbagens) isolated by these procedures and the polynucleotide sequences encoding them. In this context, the term “perturbagen” or “phenotypic probe” refers to any compound that is proteinaceous in nature and is, through its interaction with specific cellular target(s) or other such component(s), capable of disrupting or activating a particular signaling pathway and/or cellular event. As one of ordinary skill appreciates, agents may be described by their RNA sequence, amino acid sequence, or correlative DNA sequence. Alternatively, the agents can be sufficiently described in terms of their identity as isolates of a library that exhibit a particular biological activity.

Perturbagens may be encoded by a variety of genetic libraries, including those developed from cDNA, gDNA, and random, synthetic oligonucleotides synthesized using current available methods in chemistry (see, for example, Caponigro et al. (1998) “Transdominant genetic analysis of a growth control pathway.” PNAS 95:7508-7513; Caruthers, M. H. et al. (1980) Nucleic Acids Symposium, Ser. 7:215-223; Horn, T. et al. (1980) Nucleic Acids Symposium, Ser. 7:225-232; Cwirla, S. E. et al. (1990) “Peptides on phage: a vast library of peptides for identifying ligands.” Proc Natl Acad Sci 87(16):6378-82). The perturbagen itself, or fragments of the perturbagen, can be synthesized using chemical methods. For example, peptide and RNA synthesis can be performed using various techniques (Roberge, J. Y. et al. (1995) “A strategy for a convergent synthesis of N-linked glycopeptides on a solid support.” Science 269:202-204; Zhang, X. et al. (1997) “RNA synthesis using a universal base-stable allyl linker.” NAR 25(20): 3980-3983) and diverse combinatorial peptide libraries can be constructed using, a variety of strategies including but not limited to the multipin strategy, the tea bag method, or the split-couple-mix method (see, for instance, Geysen, H. M. et al (1984) “Use of peptide synthesis to probe viral antigens for epitopes to a resolution of a single amino acids.” PNAS 81:3998-4002; Houghten, R. A. (1985) “General methods for the rapid solid phase synthesis of large numbers of peptides: specificity of antigen-antibody interaction at the level of individual amino acids.” PNAS 82:5131-5135; Lam, K. S. et al. (1991) “A new type of synthetic library for identifying ligand binding activity.” Nature 354:82-84; Al-Obeidi, F. et al. (1998) “Peptide and Peptidomimetic Libraries.” Molecular Biotechnology: 9:205-223). Automated synthesis may be achieved using commercially available equipment such as the ABI 431A peptide synthesizer (Perkin-Elmer).

In some cases the polynucleotide sequence encoding a perturbagen represents a fragment of an existing gene. Using currently available software, it is possible to identify the full length cDNA by aligning the perturbagen encoding sequence with pre-existing sequences maintained in, for instance, publicly available genomic and/or EST data bases. In situations where the gene has not been identified, the perturbagen can be readily used to “reverse engineer” and identify the gene from which the phenotypic probe is derived. In this context, the term “gene” includes both the coding and antisense strands, the 5′ and 3′ regions that are not transcribed but serve as transcriptional control domains, and transcribed but not expressed domains such as introns (including splice junctions), polyadenylation signals, translation initiation signals, and the like.

In the case where a perturbagen is encoded by only a portion of a particular gene, the nucleic acid sequence of such a perturbagen may be extended utilizing a partial nucleotide sequence and employing various PCR-based methods known in the art to detect upstream sequences. One such method, restriction site PCR, uses universal and nested primers to amplify unknown sequence from genomic DNA within a cloning vector (Sarkar, G. (1993) “Restriction-site PCR: a direct method of unknown sequence retrieval adjacent to a known locus by using universal primers.” PCR Methods Applic. 2:318-322). Another method, inverse PCR, uses primers that extend in divergent directions to amplify unknown sequence from a circularized template. The template is derived from restriction fragments comprising a known genomic locus and surrounding sequences (see Triglia, T. et al. (1988) “A procedure for in vitro amplification of DNA segments that lie outside the boundaries of known sequences.” NAR. 16:8186). A third method, capture PCR, involves PCR amplification of DNA fragments adjacent to known sequences in human and yeast artificial chromosome DNA (Lagerstrom, M. et al. (1991) “Capture PCR: efficient amplification of DNA fragments adjacent to a known sequence in human and YAC DNA.” PCR Methods Applic. 1:111-119). In this method, multiple restriction enzyme digestions and ligations may be used to insert an engineered double stranded sequence into a region of known sequence before performing PCR. Other methods which may be used to retrieve unknown sequences are known in the art (Parker, J. D. et al (1991) “Targeted gene walking polymerase chain reaction.” NAR. 19:3055-3060). In addition, one may use nested primers and PROMOTERFINDER libraries (Clontech, Palo Alto, Calif.) to walk genomic DNA. This procedure avoids the need to screen libraries and is useful in finding intron/exon junctions. For all PCR based methods, primers may be designed, using commercially available software such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

In one particular embodiment, the invention encompasses proteinaceous perturbagens, biologically active fragments, (N-terminal, C-terminal, or internal) or variants thereof. The term “proteinaceous perturbagen” encompasses peptides, oligo- or polypeptides, proteins, protein fragments, or protein variants. Some proteinaceous perturbagens can be as short as three amino acids in length. Alternatively, these agents can be greater than 3 amino acids but less than ten amino acids. Other agents can be greater than ten amino acids but shorter than 30 amino acids in length. Still other agents can be greater than 30 amino acids but less than 100 amino acids in length. Still other agents can be greater than 100 amino acids in length. Naturally occurring proteinaceous perturbagens (i.e. those derived from cDNA or genomic DNA) exhibit a range in size from as little as three to several hundred amino acids. In contrast, synthetic perturbagens (such as those present in a synthetic peptide library) may range in size from three amino acids to fifty amino acids in length and more preferably, from three to 20 amino acids in length, and yet more preferably, about 15 amino acids in length.

Proteinaceous perturbagens can exert their effects by multiple means. For example, a peptide may act by binding and disrupting the interactions between two or more proteinaceous entities within the cell (see FIG. 2). In another instance, perturbagen action can result from an agent having a particular enzymatic activity and expressing that activity in, for instance, i) an unregulated fashion, or ii) in a novel compartment. Alternatively, a peptide perturbagen can bind to, and disrupt translation of a particular mRNA molecule. As still another alternative, peptide perturbagens may bind to genomic DNA and disrupt gene expression by altering the ability of one or more transcription factor(s) (e.g. activators or repressors) from binding to a critical enhancer/promoter region of the regulatory region of the gene. Perturbagens can act by these means and many others to alter or disrupt one or more aspects of a cell's physiology.

Similarly, there are multiple mechanisms by which RNA molecules may act to inhibit or activate a biological pathway. In some instances, a cytotoxic RNA agent acts in an antisense mode to disrupt ribonucleic acid transcription or translation of a cellular mRNA target via hybridization to a target ribonucleic acid (Weiss, B. et al. (1999) “Antisense RNA gene therapy for studying and modulating biological processes.” Cell Mol Life Sci 55(3): 334-58). In this context the term “antisense” refers to any composition containing a nucleic acid sequence which is complementary to the “sense” strand of a particular target DNA (see, for example, Chadwick, D. R. et al. (2000) “Antisense RNA sequences targeting the 5′ leader packaging signal region of human immunodeficiency virus type-i inhibits viral replication at post-transcriptional stages of the life cycle.” Gene Therapy 7(16): 1362-8). In other instances, the RNA molecule acts along an interference pathway (i.e. RNAi, see, for instance, Brantl, S. (2002) “Antisense-RNA regulation and RNA interference.” Biochim Biophys Acta 1575(1-3): 15-25). In still other instances, RNA perturbagens may act as a RNA-PRO agents, disrupting, for instance, a particular pathway by interacting with one or more proteinaceous components (e.g. APC or TCF) of the cell (see Sengupta, D. J. (1999) “Identification of RNAs that bind to a specific protein using the yeast three-hybrid system.” RNA 5:596-601). In still other instances, RNA agents may act as a triplex-forming oligonucleotide (TFO) agent to interact with promoter sequences, exons, introns, or other portions of genomic DNA to disrupt or activate transcription of components in a given pathway (see Postel, E. H. et al. (1989) “Evidence that a triplex-forming oligonucleotide binds to the c-myc promoter in HeLa cells, thereby reducing c-myc RNA levels.” PNAS 88: 8227-8231; Svinarchuk, F. et al. (1997) “Recruitment of transcription factors to the target site by triplex-forming oligonucleotides.” NAR 25:3459-3464).

Penetrance is another property of perturbagens. Penetrance is defined as the number of cells exhibiting a particular phenotype divided by the total number of cells in the experiment (when a perturbagen is present in the cells), minus the total number of cells exhibiting a particular phenotype divided by the total number of cells in the experiment when the perturbagen is not present in the cells. The penetrance of any given pertubagen can vary depending upon a variety of parameters including 1) the cell type in which it is being expressed, 2) the vector being used to express the perturbagen, 3) the biological stability (half-life) of the perturbagen or mRNA encoding the perturbagen and 4) the concentration of the perturbagen in the cell, as well as other parameters. In addition, the penetrance of a given perturbagen may not be directly related to the “quality” or “usefulness” of the perturbagen molecule. Thus although penetrance is a factor that impacts how immediately a given perturbagen can be seen to exert an effect, in some instances, a desirable, biologically active perturbagen may present a relatively low rate of penetrance. Furthermore, the penetrance of a given perturbagen may not be directly related to the “quality” of the molecular target it identifies.

As one of ordinary skill will appreciate, perturbagens of low penetrance may be obtained and manipulated via standard cycling and/or amplification procedures. Thus, some preferred perturbagens might exhibit as low as 1-2% penetrance. Other preferred perturbagens may exhibit between 2% and 5% penetrance, between 5 and 10% penetrance, 10% and 20% penetrance, between 20% and 50% penetrance, or even in some instances, between 50% and 100% penetrance.

In some instances, the action, penetrance, or biological activity of a perturbagen may be affected in some part by the scaffold to which it is associated. In some cases (for instance, in situations where the agent is shorter than 30 amino acids) the scaffold may drive the perturbagen to adopt a conformation that enhances its biological action. In still other instances, one or more neighboring residues from, e.g., the C-terminus of a scaffold, may act in concert with the perturbagen to enhance the functionality of the molecule. In cases such as these, the complete biologically active sequence may include one or more C-terminal residues derived from the scaffold molecule. Multiple techniques may be used to determine the contribution of the scaffold to the phenotypic effect of any given perturbagen. Initially, perturbagen sequences can be shifted to alternative scaffolds and retested for biological activity. If these procedures result in a significant loss of the perturbagen's activity, a fusion between the perturbagen and, for instance, the 30-most residues from the C-terminus of the original scaffold may be linked to a second scaffold molecule and retested for biological activity. Should operations such as these lead to the recovery of lost activity, experiments in which smaller and small portions of the scaffold are associated with the perturbagen can be tested.

Perturbagens may also exhibit cross-reactivity. A variety of host target proteins can contain similarities in both the primary and secondary structure. As a result, one or more of the agents described herein may exhibit affinity for one or more target variants/isoforms present in nature. Similarly, agents identified in the following screens may exhibit affinity for two or more functionally unrelated proteins that contain regions or domains that share homology or related functional groups. Thus, for instance, a perturbagen that recognizes a zinc-binding domain of one protein may also show affinity for the homologous (and functionally equivalent) region of a second protein (see, e.g. Mavromatis K. O. et al. (1997) “The carboxyl-terminal zinc-binding domain of the human papillomavirus E7 protein can be functionally replaced by the homologous sequences of the E6 protein.” Viral Research 52(1): 109-18). In cases where such interactions lead to relevant biological phenotypes, the underlying mechanism(s) may differ considerably from those brought about by the original perturbagen-target interactions. Furthermore, in cases where an agent exhibits cross reactivity with secondary targets, said agents may be useful in a broader set of therapeutic and diagnostic applications than originally intended.

Host range is another characteristic of perturbagens. The term “host range” refers to the breadth of potential host cells that exhibit perturbagen-induced phenotypes. In some instances, such as the case where, for instance, an agent is represented by an apoptosis-inducing fragment of BID, the host range is broad, due to the near ubiquitous participation of BID or BID-like agents in the apoptotic pathway of many cells. In contrast, some perturbagens have a very limited host range. For instance, a particular perturbagen may induce a desired physiological response due to, for instance, the restricted expression of the perturbagen target to a limited number of cells (e.g. colon cells). Alternatively, in some cases, the perturbagen may recognize a target that is a mutated molecule that is only found in, for instance, a cancer cell. In these instances, the phenotype induced by the perturbagen is restricted to cells that contain that specific mutated target or cells that have, for instance, mutations that are similar in their chemical nature and lead to similar molecular function, conformational changes, or physiological outcomes.

2. Sequence Variants

In another embodiment, the invention includes sequence variants of both the phenotypic probes and the polynucleotide sequences that encode them. In the case of proteinaceous perturbagens, variants contain at least one amino acid substitution, deletion, or insertion from the original isolated form of the perturbagen that provides biological properties that are substantially similar to those of the initial perturbagen. Similarly, variants of RNA-based phenotypic probes contain at least one nucleotide substitution, deletion, or insertion when compared to the original isolated sequence. Variants may occur in the RNA/DNA that encodes each phenotypic probe and thus it is possible for each to contain at least one nucleotide substitution, deletion, or insertion when compared to the original isolated sequence.

In addition to being described by their respective sequence, variants may also be identified by the relative amounts of homology they have in common with the original perturbagen sequence. “Homology” is defined as the percentage of residues in a candidate sequence that are identical with the residues in the reference sequence after aligning the two sequences and introducing gaps, if necessary, to achieve the maximum percent of overlap (see, for example, Altschul, S. F. et al. (1990) “Basic local alignment search tool.” J Mol Biol 215(3): 403-10; Altschul, S. F. et al. (1997) “Gapped BLAST and PSI-BLAST: a new generation of protein database search programs.” Nucleic Acids Res 25(17): 3389-402).

Alternatively, a variant of a proteinaceous perturbagen may be described in terms of the nature of an amino acid substitution. “Conservative” substitutions are those in which the substituting residue is structurally or functionally similar to the substituted residue. In non-conservative substitutions, the substituting and substituted residue will be from structurally or functionally different classes. For the purposes herein, these classes are as follows: 1. Electropositive: R, K,H; 2. Electronegative: D,E; 3. Aliphatic: V,L,I,M; 4. Aromatic: F,Y,W; 5. Small: A,S,T,G,P,C; 6. Charged: R,K,D,E,H; 7. Polar: S,T,Q,N,Y,H,W; and Small Hydrophilic: C,S,T. Interclass substitutions generally are characterized as nonconservative, while intraclass substitutions are considered to be conservative. In some instances, variant polypeptides sequences can have 65-75% homology with the original agent. In other embodiments, variants have between 75% and 85% homology with the original agent. In still other embodiments, variants will have between 85% and 95% homology with the original perturbagen agent. In yet other embodiments, variants have between 95% and greater than 99% polypeptide sequence identity with the original perturbagen agent. In some cases, the homology between two perturbagens (variants) is confined to a small region of the molecule (e.g. a motif). Such conserved sequences are often indicative of regions that contain biologically important functions and suggest the perturbagens share a common cellular target. In these situations, while only limited and conservative amino acid changes are desirable within the region of the motif, greater levels of variation can exist in adjacent and more distal portions of the polypeptide.

The RNA encoding each perturbagen (or RNA perturbagens) may also be described in terms of percent homology. In some instances, the variant ribonucleotide sequences can have 65-75% homology with the original agent. In other embodiments, the variants have between 75% and 85% homology with the original agent or between 85% and 95% homology with the original perturbagen sequence, or even between 95% and greater than 99% sequence identity with the original perturbagen agent. Again, greater variation can, in some embodiments, exist outside an identified region/motif without altering biological activity.

Lastly, in reference to the DNA sequences encoding proteinaceous perturbagens, one who is skilled in the art will appreciate that the degree of variance will depend upon and/or reflect the degeneracy of the genetic code. As one in the art appreciates, a given protein sequence is equivalently encoded by a large number of polynucleotide sequences. Therefore, the invention encompasses each variation of polynucleotide sequence that encodes the given perturbagen, such variations being made in accordance with the standard triplet genetic code as applied to the polynucleotide sequence of each perturbagen. For each proteinaceous perturbagen described by amino acid sequence herein, all such corresponding DNA variations are to be considered as being specifically disclosed.

Variants of phenotypic probes may arise by a variety of means. Some variants may be artifactual and result from, for instance, errors that occur in the process of PCR amplification or cloning of the perturbagen encoding sequence. Alternatively, variants may be constructed intentionally. For instance, it may be advantageous to produce nucleotide sequences encoding perturbagens possessing a substantially different codon usage. Codons may be selected to increase the rate at which expression of the peptide or RNA occurs in a particular prokaryotic or eukaryotic cell in accordance with the frequency with which particular codons are utilized by the host (Berg, O. G. (1997) “Growth rate-optimized tRNA abundance and codon usage.” J Mol Biol 270(4): 544-50). Additional reasons for substantially altering the nucleotide sequence encoding proteinaceous perturbagens (without altering the encoded amino acid sequences) include, but are not limited to, producing RNA transcripts that have increased half-life. This may be accomplished by altering a sequence's structural stability (see, for example, Gross, G. et al. (1990) “RNA primary sequence or secondary structure in the translational initiation region controls expression of two variant interferon-beta genes in Escherichia coli.” J Biol Chem. 265(29): 17627-36; Ralston, C. Y. et al. (2000) “Stability and cooperativity of individual tertiary contacts in RNA revealed through chemical denaturation.” Nat Struct Biol. 7(5): 371-4), or through addition of untranslated sequences that increase RNA stability/half-life through RNA-protein interactions (see, for example, Wang, W. et al. (2000) “HuR regulates cyclin A and cyclin B1 mRNA stability during cell proliferation.” EMBO J. 19(10): 2340-50; Shetty, S. and Idell, S. (2000) “Posttranscriptional regulation of plasminogen activator inhibitor-1 in human lung carcinoma cells in vitro.” Am J Physiol Lung Cell Mol Physiol 278(1): L148-56). Also included the category of “intentional variants” are those whose sequence has been altered in order to add or deleted sites involved in post-translational modification. Included in this list are variants in which phosphorylation sites, acetylation sites, methylation sites, and/or glycosylation sites have been added or deleted (see, for example, Wicker-Planquart, C. (1999) “Site-directed removal of N-glycosylation sites in human gastric lipase.” Eur J Biochem. 262(3): 644-51; Dou, Y. (1999) “Phos-phorylation of linker histone Hi regulates gene expression in vivo by mimicking Hi removal.” Mol Cell. 4(4): 641-7).

Variants may also arise as a result of simple and relatively routine techniques involving random mutagenesis or “DNA shuffling”; procedures that are often used to rapidly evolve perturbagen encoding sequences and allow identification of variants that have increased biological stability or activity (see, for instance, Ner, S. S. et al. (1988) “A simple and efficient procedure for generating random point mutations and for codon replacements using mixed oligonucleotides.” DNA 7:127-134; Stemmer, W. (1994) “Rapid evolution of a protein in vitro by DNA shuffling.” Nature 370:389-391). For instance, in mutagenic PCR, the fragment encoding the perturbagen is PCR amplified under conditions that increase the error rate of Taq polymerase. This may be accomplished by i) increasing the MgCl₂ concentrations to stabilize non-complementary pairings, ii) addition of MnCl₂ to diminish template specificity of the polymerase and iii) increasing the concentration of dCTP and dTTP to promote misincorporation of basepairs in the reaction. As a result of this process, the error rate of Taq polymerase may be increased from 1.0×10⁻⁴ errors per nucleotide per pass of the polymerase, to approximately 7×10⁻³ errors per nucleotide per pass. Amplifying a perturbagen-encoding sequence under these conditions allows the development of a library of dissimilar sequences that can subsequently be screened for variants that exhibit improved biological activity.

In addition to variants that are created by artificial or accidental means, natural variants may also exist. For instance, in the course of screening any given genomic or cDNA library, it is possible that a perturbagen, derived from a sequence that exists in multiple copies within the genome (e.g. duplications, repetitive sequences), may be isolated numerous times. Such sequences often contain polymorphisms that result in alterations in the encoded RNA and polypeptide sequence (see, for example, Satoh, H. et al. (1999) “Molecular cloning and characterization of two sets of alpha-theta genes in the rat alpha-like globin gene cluster.”Gene 230(1): 91-9) and thus, may represent natural variants of the perturbagen agent. Alternatively, if multiple libraries are utilized to screen for perturbagens and two or more of those libraries are derived from unrelated individuals, dissimilar tissues, or contrary periods in the development of a tissue (e.g., adult vs. fetal tissue), it is possible that variants may be isolated as a result of allelic or splice variation respectively (see, for example, Posnett, D. N. (1990) “Allelic variations of human TCR V gene products.” Immunol Today. 11(10): 368-73). Variants of phenotypic probes may arise by these and other means.

Variants of any given perturbagen may in some instances exhibit additional biological properties. For instance, perturbagens that previously recognized only a single target may demonstrate broadened specificity, e.g., may bind multiple isoforms or serotypes of a target in response to the alteration of a single amino acid in the perturbagen variant. Similarly, a perturbagen having a specific phenotype in one cell may exhibit a greater or lesser collection of phenotypes or may exhibit a broader or more restricted effective host range after making small alterations in perturbagen variant sequence.

3. Biologically Active Fragments

Some embodiments of the invention encompass “biologically active fragments” of a given proteinaceous perturbagen. In this context, the term “fragment” refers to any portion of a proteinaceous perturbagen that is at least 3 amino acids in length, and the corresponding nucleic acids that encode such fragments. The terms “biologically relevant” or “biologically active” refer to that portion of a protein or protein fragment, RNA or RNA fragment, or DNA fragment that encodes either of the two previous entities, that is responsible for an observable phenotype (or for activation of a correlative reporter construct). Thus, biologically active fragments may be comprised of N-terminal, C-terminal, or internal fragments of peptide perturbagens. In some instances, the fragment encodes or represents portions of a natural gene. In other instances the fragment is derived from a larger polynucleotide or polypeptide that has no known natural counterpart. In still other instances, biologically active regions of a perturbagen can be artificially synthesized (by chemical or recombinant methods) so that multiple, tandem copies of the phenotypic probe are covalently linked together and expressed. All such biologically active perturbagen fragments are, in turn, encoded by a variety of correlative DNA sequences.

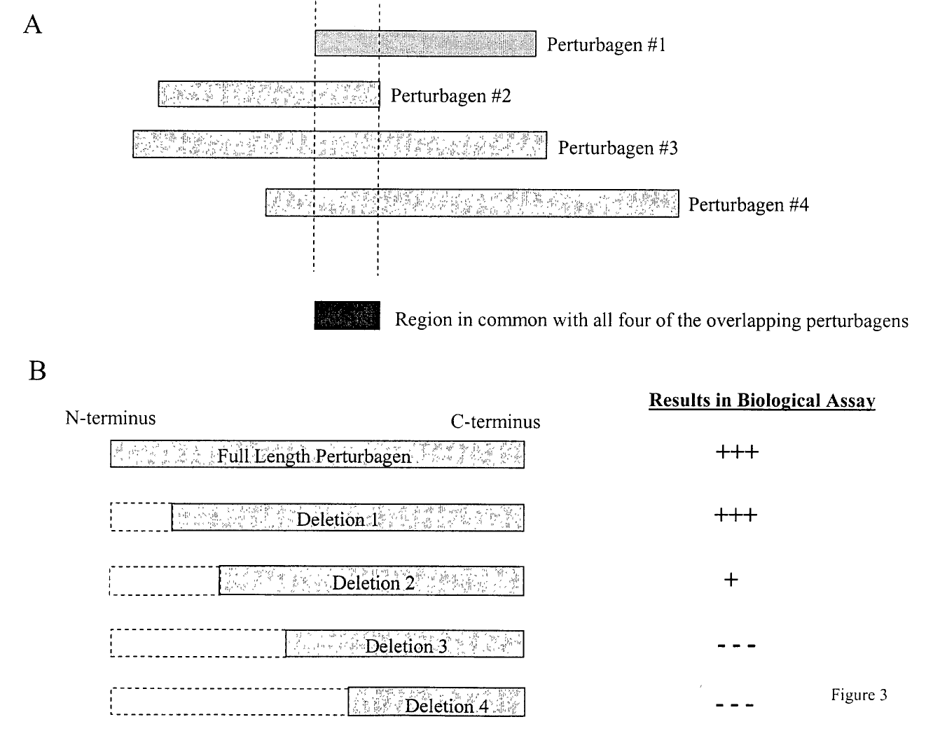

The biologically active portion of a molecule can be identified by several means. In some instances, biological relevant regions can be deduced by simple physical mapping of families of overlapping sequences isolated from a phenotypic assay (Hingorani, K. et al. (2000) “Mapping the functional domains of nucleolar protein B23.” J Biol Chem May 26). For instance, in the course of any given screen, multiple perturbagens, derived from alternative breakpoints of the same gene, may be isolated from one or more genetic libraries (FIG. 3). The smallest region that is common to all of the perturbagens can demarcate the area of biological importance.

Alternatively, critical regions of a perturbagen can frequently be distinguished by comparing the polynucleotide and/or amino acid sequence of two or more perturbagens that share a common target (see, for example, Grundy, W. N. (1998) “Homology detection via family pair-wise search.” J Comput Biol. 5(3):479-9; Gorodkin, J. et al. (1997) “Finding common sequence and structure motifs in a set of RNA sequences.” Ismb 5:120-3). In this instance, conserved sequences (or motifs) that are identified by this form of analysis often provide important clues necessary to determine biologically important regions of a given molecule. Alternatively, methods that identify biologically relevant regions by altering or deleting regions of the perturbagen molecule can also be used. For instance, the gene encoding a particular perturbagen can be subjected to deletion analysis whereby portions of the gene are removed in a systematic fashion, thus allowing the remaining entity to be retested for its ability to evoke a biological response (see, FIG. 3 and Huhn, J. et al. (2000) “Molecular analysis of CD26-mediated signal transduction in cells.” Immunol Lett 72(2):127-132; Davezac, N. et al. (2000) “Regulation of CDC25B phosphatases subcellular localization.” Oncogene 19(18): 2179-85).

Alternatively, biologically critical regions of a molecule can be identified by inducing mutations in the sequence encoding the polypeptide (see, for example, Ito, Y. et al. (1999) “Analysis of functional regions of YPM, a superantigen derived from gram-negative bacteria.” Eur J Biochem; 263(2): 326-37; Kim, S. W. et al. (2000) “Identification of functionally important amino acid residues within the C2-domain of human factor V using alanine-scanning mutagenesis.” Biochemistry 39(8): 1951-8.). Subsequent testing of the variants of said molecule for biological activity enables the identification of regions of the perturbagen that are both critical and sensitive to manipulation. Molecular probes such as monoclonal antibodies and epitope-specific peptides can also be useful in the identification of biologically important regions of a perturbagen (see, for example, Midgley, C. A. et al. (2000) “An N-terminal p14ARF peptide blocks Mdm2-dependent ubiquitination in vitro and can activate p53 in vivo.” Oncogene 19(19): 2312-23; Lu, D. et al. (2000) “Identification of the residues in the extracellular region of KDR important for interaction with vascular endothelial growth factor and neutralizing anti-KDR antibodies.” J Biol Chem 275(19): 14321-30). In this procedure, probes that bind and thus mask specific regions of a perturbagen can be tested for their ability to block the biological activity of the molecule. These techniques (as well as others) can be used to map the boundaries of any given biologically active residues.

4. Heterologous Sequences

In another embodiment, the invention encompasses all heterologous forms of the phenotypic probes and the polynucleotide sequences encoding them described herewith. In this context, “heterologous sequence(s)” include versions of the perturbagens that are i) scaffolded by other entities, ii) tagged with marker sequences that can be recognized by antibodies or specific peptides, iii) altered to transform post-translational patterns of modification or iv) altered chemically so as to cyclicize the molecule for alternative pharmacodynamic/pharmacokinetic properties.

The term “scaffold” refers to a proteinaceous or RNA sequence to which the perturbagen or perturbagen encoding ribonucleic acid sequence is covalently linked during synthesis to provide e.g., conformational stability and/or protection from degradation. Thus peptide perturbagens can be fused to protein scaffolds at N-terminal, C-terminal, or internal sites. Similarly, the RNA sequences encoding those perturbagens can be fused to RNA sequences at 5′, 3′ or internal sites and increase the stability of the messenger RNA (mRNA) of said agent.

In some instances, scaffolds may be a relatively inert protein, (i.e. having no enzymatic activity or fluorescent properties). As one example of an inert scaffold, we created a non-fluorescent variant of GFP called dead-GFP (also referred to as “dGFP”). In this case, the nonfluorescent variant arises from conversion of Tyr→Phe at codon 66 of EGFP. Such proteins can be stably expressed in a wide variety of cell types without disrupting the normal physiological functions of the cell. In addition, such chimeric fusions can easily be detected by Western Blot analysis using antibodies directed against GFP and are useful in determination of intracellular expression levels of perturbagens. In other instances, scaffolds may serve a dual function, e.g., increasing perturbagen stability while at the same time, serving as an indicator or gauge of the level of perturbagen expression. In this case, the scaffold may be an autofluorescent molecule such as a green fluorescent protein (GFP, Clontech), ZsYellow, or ZsGreen, or embody an enzymatic activity capable of altering a substrate in such a way that it can be detected by eye or instrumentation (e.g. β galactosidase). Lastly, by modifying the perturbagen sequences or the scaffold to which they are attached with various “localization” signals, the perturbagen may be directed to a particular compartment within the host cell. For example, proteinaceous perturbagens can be directed to the nucleus of certain cell types by attachment of a nuclear localization sequence (NLS); a heterogeneous sequence made up of short stretches of basic amino acid residues recognized by importins alpha and/or beta (see, for example, Lobl, T. J. et al. (1990) SV40 large T-antigen nuclear signal analogues: successful nuclear targeting with bovine serum albumin but not low molecular weight fluorescent conjugates.” Biopolymers 29(1): 197-203).

Perturbagens can be constructed to contain a heterologous moiety (a “tag”) that is recognized by a commercially available antibody. Such heterologous forms may facilitate studies of subjects including, but not limited to, i) perturbagen subcellular localization, ii) intracellular concentration assessment and iii) target binding interactions. In addition, the tagging of a perturbagen may also facilitate purification of fusion proteins using commercially available matrices (see, for example, James, E. A. et al. “Production and characterization of biologically active human GM-CSF secreted by genetically modified plant cells.” Protein Expr Purif. 19(1): 131-8; Kilic, F. and Rudnick, G. (2000) “Oligomerization of serotonin transporter and its functional consequences.” Proc Natl Acad Sci U S A. 97(7): 3106-11). Such tagged moieties include, but are not limited to glutathione-S-transferase (GST), maltose binding protein (MBP), thioredoxin (Trx), calmodulin binding peptide (CBP), 6-H is, FLAG, c-myc, and hemagglutinin (HA). GST, MBP, Trx, CBP, and 6-H is enable purification of their cognate fusion proteins on immobilized glutathione, maltose, phenylarsine oxide, calmodulin, and metal-chelate resins, respectively. FLAG, c-myc and HA enable immunoaffinity purification of fusion proteins using commercially available monoclonal and polyclonal antibodies that specifically recognize these epitope tags. Such fusion proteins may also be engineered to contain a proteolytic cleavage site located between the perturbagen sequence and the heterologous protein sequence, so that the perturbagen may be cleaved away from the heterologous moiety following purification. A variety of commercially available kits may be used to facilitate expression and purification of fusion proteins.

An additional embodiment of the invention includes antibodies that recognize the perturbagen itself or cellular targets of the perturbagen. Antibodies directed against perturbagens or cellular targets may be useful for a variety of purposes including i) therapeutics, ii) diagnostic assays iii) immunocytochemistry, iv) target identification, and v) purification. Such reagents may include, but are not limited to, polyclonal, monoclonal, chimeric, and single chain antibodies, Fab fragments, and fragments produced by a Fab expression library. For the production of antibodies, various hosts including goats, rabbits, rats, mice, humans and others may be immunized by injection with a perturbagen or any fragment thereof that has immunogenic properties. Depending on the host species, various adjuvants may be used to increase immunological response. Such adjuvants include, but are not limited to Freund's, mineral gels such as aluminum hydroxide, and surface-active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Monoclonal antibodies that recognize perturbagens may be prepared using any technique that provides for the production of antibody molecules by continuous cell lines in culture. These include, but are not limited to, the hybridoma technique, the human B-cell hybridoma technique, and the EBV hybridoma technique. (see, for example, Kohler, G. et al. (1975) “Continuous cultures of fused cells secreting antibody of predefined specificity.”Nature 256:495-497; Kozbor, D. et al (1985) “Specific immunoglobulin production and enhanced tumorigenicity following ascites growth of human hybridomas.” J. Immunol. Methods 81:31-42; Cote, R. J. et al. (1983) PNAS 80:2026-2030; and Cole, S. P. et al. (1984) “Generation of human monoclonal antibodies reactive with cellular antigens” Mol. Cell Biol. 62:109-120).

In addition, techniques developed for the production of chimeric antibodies, such as the splicing of mouse antibody genes to human antibody genes to obtain a molecule with appropriate antigen specificity and biological activity can be used. See, e.g., Morrison, S. L. et al. (1984) “Chimeric human antibody molecules: mouse antigen-binding domains with human constant region domains.” PNAS 81:6851-6855); Neuberger, M. S. et al. (1984) “Recombinant antibodies possessing novel effector functions.” Nature 312:604-608; and Takeda, S. et al. (1985) “Construction of chimeric processed immunoglobulin genes containing mouse variable and human constant region sequences.” Nature 314:452-454). Alternatively, techniques described for the production of single chain antibodies may be adapted, using methods known in the art, to produce perturbagen-specific antibodies (see, e.g. Burton, D. R. (1991) “A large array of human monoclonal antibodies to type 1 human immunodeficiency virus from combinatorial libraries of asymptomatic seropositive individuals.” PNAS 88:10134-10137).

Antibodies may also be produced by inducing in vivo production in the lymphocyte population or by screening immunoglobulin libraries or panels of highly specific binding reagents as disclosed in the literature. (see, for example, Orlandi, R. et al. (1989) “Cloning immunoglobulin variable domains for expression by the polymerase chain reaction.” PNAS 86:3833-3837; Winter, G. et al. (1991) “Man-made antibodies.” Nature 349: 293-299).

Antibody fragments that contain specific binding sites for perturbagens may also be generated. For example, such fragments include, but are not limited to F(ab′)₂ fragments produced by pepsin digesting of the antibody molecule and Fab fragments generated by reducing the disulfide bridges of the F(ab′)₂ fragments. Alternatively, Fab expression libraries may be constructed to allow rapid and easy identification of monclonal Fab fragments with the desired specificity. (See, for example, Huse, W. D. et al. (1989) “Generation of a large combinatorial library of the immunoglobulin repertoire in phage lambda.” Science 246:1275-1281).

In addition to the chimeric variants described above, chemical modification encompasses a variety of modifications including, but not limited to, perturbagens that have been radiolabeled with ³²P or ³⁵S, acetylated, glycosylated, or labeled with fluorescent molecules such as FITC or rhodamine. These modifications may be directly imposed on the perturbagen itself (see, for example, Shuvaev, V. V. et al. (1999) “Glycation of apolipoprotein E impairs its binding to heparin: identification of the major glycation site.”Biochim Biophys Acta 1454(3):296-308; Dobransky, T. et al. (2000) “Expression, purification and characterization of recombinant human choline acetyltransferase: phosphorylation of the enzyme regulates catalytic activity.” Biochem J. 349(Pt 1): 141-151). Alternatively, changes may be made to the polynucleotide sequence encoding the perturbagen so as to alter the pattern of phosphorylation, acetylation, or glycosylation. In addition, the term “chemical modification” may include methods that lead to cyclization of peptides in order to alter membrane permeability and/or pharmacodynamic-pharmacokinetic properties (see, for example, Borchardt, R. T. (1999) “Optimizing oral adsorption of peptides using prodrug strategies.” J Control Release 62(1-2): 231-8.).

5. Hybridization

The invention also encompasses polynucleotide sequences that are capable of hybridizing to the claimed polynucleotide sequences encoding phenotypic probes and said variants of such entities described previously, under various conditions of stringency and which encode a perturbagen with the same or similar biological activity. Such reagents may be useful in i) therapeutics, ii) diagnostic assays, iii) immunocytology, iv) target identification, and v) purification. For example, if the sequence encoding a particular perturbagen is introduced into a subject for gene therapeutic purposes, it may be necessary to monitor the success of integration and the levels of expression of said agent by Southern and Northern Blot analysis respectively (Pu, P. et al. (2000) “Inhibitory effect of antisense epidermal growth factor receptor RNA on the proliferation of rat C6 glioma cells in vitro and in vivo.” J Neurosurg. 92(1): 132-9). In other instances, hybridization may be used as a tool to define or describe a perturbagen variant or fragment, and a hybridizing sequence thus may have direct relevance as a mimetic or other such therapeutic agent.

The term “hybridization” refers to any process by which a strand of nucleic acid binds with a complementary or near-complementary strand through base pairing. There are several parameters that play a role in determining whether two polynucleotide molecules will hybridize including salt concentrations, temperature, and the presence or absence of organic solvents. For instance stringent salt concentrations will ordinarily be less than about 750 mM NaCl and 75 mM trisodium citrate, preferably less than about 500 mM NaCl and 50 mM trisodiium citrate, and most preferably less than about 250 mM NaCl and 25 mM trisodium citrate. Low stringency hybridization can be obtained in the absence of organic solvent (e.g. formamide) while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent and the inclusion or exclusion of carrier DNA are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide and 100 ug/ml denatured salmon sperm DNA (ssDNA). In a most preferred embodiment, hybridization will occur at 42° C. in 250 mM NaCl, 25 mM trisodium citrate, 1% SDS, 50% formamide and 200 ug/ml denatured ssDNA. Useful variations on these conditions will be readily apparent to those skilled in the art.

The washing steps that follow hybridization can also vary greatly in stringency. Wash stringency conditions can be defined by salt concentration and by temperature. As above, wash stringency can be increased by decreasing salt concentration or by increasing temperature. For example, stringent salt concentrations for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include temperatures of at least about 25° C., more preferably of at least about 42° C., and most preferably of at least about 68° C. In a preferred embodiment, wash steps will occur at 25° C. in 30 mM NaCl, 3 mM trisodium citrate and 0.1% SDS. In a more preferred embodiment, wash steps will occur at 42° C. in 15 mM NaCl, 1.5 mM trisodium citrate and 0. 1% SDS. In a most preferred embodiment, wash steps will occur at 68° C. in 15 mM NaCl, 1.5 mM trisodium citrate and 0.1% SDS. Additional variations on these conditions will be readily apparent to those skilled in the art.

M. Databases

The compositions, relations and phenotypic effects yielded by the methodology described herein may advantageously be placed into or stored in a variety of databases. As one example, a database may include information about one or more targets identified by the methods herein, including for example sequence information, motif information, structural information and/or homology information. The database may optionally contain such information regarding perturbagen agents, and may correlate the perturbagen information to corresponding target information. Further helpful database aspects may include information regarding, e.g., variants or fragments of the above. The database may also correlate the indexed compounds to, e.g., immunoprecipitation data, further yeast n-hybrid interaction data, genotypic data (e.g., identification of disrupted genes or gene variants), and with a variety phenotypic data. Such databases are preferably electronic, and may additionally be combined with a search tool so that the database is searchable

N. Therapeutic Uses

Natural and synthetic chemotherapeutic derivatives have proven valuable in the treatment of a variety of forms of disease. For that reason, in one embodiment, perturbagens, fragments or derivatives of a perturbagen, small molecule mimetics of a perturbagen, sequences encoding perturbagens, sequences that can hybridize to perturbagen encoding sequences, targets of the perturbagen, or agents that bind said target (e.g. antibodies) or portions thereof, may be utilized to treat or prevent a disorder that has previously shown sensitivity to treatment with chemotherapeutics and/or radiation therapy. Thus, for example, polypeptides or RNA molecules described herein can be used i) modulate cellular proliferation, ii) modulate cellular differentiation, iii) induce or modulate necrotic or apototic processes, or iv) sensitize cells to secondary compounds that induce either i), ii), or iii) by direct application of said agent. Examples of such disorders that may be aided by such agents include, but are not limited to cancers of the i) ovary, ii) liver, iii) endometrium, iv) stomach, colon and/or rectum, v) prostrate, vi) uterus, vii) esophagus, viii) kidney, ix) thyroid, x) stomach, xi) brain, xii) skin and xiii) breast.

Ailments such as those described previously can be treated with the perturbagen directly or indirectly. Thus either a purified form of the perturbagen can be administered to the patient or a vector capable of expressing a perturbagen or a fragment or derivative thereof may be administered to a subject to treat or prevent a disease. Expression vectors including, but not limited to, those derived from retroviruses, adenoviruses, adeno-associated viruses, or herpes or vaccinia viruses or from various bacterial plasmids, may be used for delivery of nucleotide sequences to the targeted organ, tissue, or cell population (see, for example, Carter, P. J. and Samulski, R. J. (2000) “Adeno-associated viral vectors as gene delivery vehicles.” Int J Mol Med. 6(1):17-27; Palu, G. et al. (2000) “Progress with retroviral gene vectors.” Rev Med Virol. 10(3):185-202; Wu, N. and Ataai, M. M. (2000) “Production of viral vectors for gene therapy applications.” Curr Opin Biotechnol. 11(2): 205-8).

In a further embodiment, a pharmaceutical composition comprising a substantially purified perturbagen, or a fragment thereof, or a small molecule mimetic, optionally in conjunction with a suitable pharmaceutical carrier, may be administered to a subject to treat or prevent any of the previously mentioned disorders. As used herein, the language “pharmaceutical carrier” is intended to include any and all solvents, dispersion media, coatings, antibacterial and antifungal agents, and the like, compatible with pharmaceutical administration. The use of such media and agents for pharmaceutically active substances is well known in the art. Except insofar as any conventional media or agent is incompatible with the active compound, use thereof in the compositions is contemplated.

Pharmaceutical compositions of the invention are formulated to be compatible with intended routes of delivery. Examples of routes of administration include parenteral e.g. intravenous, intradermal, subcutaneous, oral, inhalation, transdermal, topical, transmucosal, and rectal administration. Solutions or suspensions used for parenteral, intradermal, or subcutaneous application can include the following components: a sterile diluent, such as water for injection, saline solution, fixed oils, polyethylene, glycols, glycerine, propylene glycol, or other synthetic solvents, antibacterial agents such as benzyl alcohol or methyl parabens, antioxidants such as ascorbic acid or sodium bisulfite, chelating agents such as ethylenediaminetetraacetic acid, buffers such as acetates, citrates, or phosphates and agents for the adjustment of tonicity such as sodium chloride or dextrose.

Pharmaceutical compositions suitable for injectable use include aqueous solutions (where water-soluble) or dispersions and sterile powders for the extemporaneous preparation of sterile injectable solutions or dispersions. For intravenous administration, suitable carriers include physiological saline, bacteriostatic water Cremophor EL™ (BASF; Parsippany, N.J.) or phosphate buffered saline (PBS). In all cases the composition must be sterile and should be fluid to the extent that easy syringability exists. Oral compositions can also be prepared using any of the following ingredients, or compounds of a similar nature: a binder such as microcrystalline cellulose, gum tragacanth, or gelatin; an excipient such as starch or lactose, disintegrating agent such as alginic acid, Primogel, or corn starch; a lubricant such as magnesium stearate, a glidant such as colloidal silicon dioxide, a sweetening agent such as sucrose or saccharin, or a flavoring agent such as peppermint or orange flavoring. For administration by inhalation, the compounds are delivered in the form of an aerosol spray from a pressurized container or dispenser that contains a suitable propellant. Systemic administration can also be by transmucosal or transdermal means. For these methods of administration, penetrants appropriate to the barrier to be permeated are used in the formulation. Such penetrants are generally known in the art and include, for example, bile salts and fusidic acid derivatives. Transmucosal administration can also be accomplished through the use of nasal sprays and suppositories. For transdermal administration, the active compounds are formulated into ointments, salves, gels, or creams as generally known in the art.

In one embodiment, the active compounds are prepared with carriers that will protect the compound against rapid elimination from the body, such as a controlled microencapsulated delivery system. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to specific cell surface epitopes) can also be used as pharmaceutically acceptable carriers. These can be prepared according to methods known to those skilled in the art, for example, as described in U.S. Pat. No. 4,522,811.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, for example, Chen, S. H. et al. (1994) “Gene therapy for brain tumors: regression of experimental gliomas by adenovirus-mediated gene transfer in vivo.” PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

O. Diagnostic Uses

The polynucleotides, polypeptides, variants, targets and antibodies to any one of these molecules can, in addition to previously mentioned therapeutic applications, be used in one or more of the following methods: 1) detection assays (e.g. chromosomal mapping, tissue typing, forensic biology), and 2) predictive medicine (e.g. diagnostic or prognostic assays, pharmacogenomics and monitoring clinical trials). Thus, for example, agents may be used to detect a specific mRNA or gene (e.g. in a biological sample) for a genetic lesion. Similarly, agents described herein may be applied to the field of predictive medicine in which diagnostic assays or prognostic assays, pharmacogenomics, and monitoring clinical trials are used for predictive purposes to thereby treat an individual prophylactically.

Accordingly, one aspect of the present invention relates to diagnostic assays for determining expression of a polypeptide or nucleic acid of the invention and/or activity of said agent of the invention, in the context of a biological sample to thereby determine whether an individual is afflicted with a disease or disorder, or is at risk of developing a disorder, associated with aberrant expression or activity of a polypeptide or polynucleotide of the invention.

Alternatively, the invention provides methods for detecting expression of a nucleic acid or polypeptide of the invention or activity of a polypeptide or polynucleotide of the invention in an individual to thereby select appropriate therapeutic or prophylactic agents for that individual (referred to herein as “pharmacogenomics”). Pharmoacogenomics allows for the selection of agents (e.g. drugs) for therapeutic or prophylactic treatment of an individual based on the genotype of the individual (e.g. the genotype of the individual examined to determine the ability of the individual to respond to a particular agent). Still another aspect of the invention pertains to monitoring the influence of agents (e.g. drugs or other compounds) on the expression or activity of a polypeptide or polynucleotide of the invention in clinical trials.

P. Detection Assays

Portions or fragments of the polynucleotide sequences of the invention can be used in numerous ways as polynucleotide reagents. For example, these sequences can be used to i) map their respective genes on a chromosome and, thus, locate gene regions associated with genetic diseases; ii) identify an individual from a minute biological sample (tissue typing); and iii) aid in forensic identification of biological samples.

1. Gene and Chromosome Mapping.

Once the sequence (or portion of a sequence) of a gene has been isolated, this sequence can be used to identify the entire gene, analyze the gene for homology to other sequences (i.e., identify it as a member of a gene family such as EGF receptor family) and then map the location of the gene on a chromosome. Accordingly, nucleic acid molecules described herein or fragments thereof, can be used to map the location of the gene on a chromosome. The mapping of the sequences to chromosomes is an important first step in correlating these sequences with genes associated with disease.

Briefly, genes can be mapped to chromosomes by preparing PCR primers from the sequence of a gene of the invention. These primers can then be used for PCR screening of somatic cell hybrids containing individual chromosomes. Only those hybrids containing the human gene corresponding to the gene sequences will yield an amplified fragment (For review of this technique se D'Eustachio, P. and Ruddle, F. H. (1983) “Somatic cell genetics and gene families.” Science 220:919-924). Alternative methods of mapping a gene to its chromosome include in situ hybridization (see, for example, Fan, Y. S. et al. (1990) “Mapping small DNA sequences by fluorescence in situ hybridization directly on banded metaphase chromosomes.” PNAS 87:6223-27), pre-screening with labeled flow sorted chromosomes (CITE), and pre-selection by hybridization to chromosome specific cDNA libraries. Furthermore, fluorescence in situ hybridization (FISH) of a DNA sequence to a metaphase chromosome spread can further be used to provide a precise chromosomal location in one step (see “Human Chromosomes: A Manual of Basic Techniques”, Pergamon Press, New York, 1988). Lastly, with the completion (in the not-to-distant future) of the sequencing of the human genome, chromosome mapping will very quickly switch from elaborate, hands-on methods of mapping genes, to simple database searches

Once the sequence (or portion of a sequence) of a gene has been isolated, these agents can be used to assess the intactness or functionality of a particular gene. Comparison of affected and unaffected individuals can begin with looking for structural alterations in the chromosomes such as deletions, inversions, or translocations that are based on that DNA sequence. Once this is accomplished, the physical position of the sequence on the chromosome can be correlated with genetic data map. (such data are found, for example in McKusick, V. “Mendialian Inheritance in Man” available on-line through John Hopkins University Welch Medical Library). The relationship between genes and disease, mapped to the same chromosomal region can then be identified through linkage analysis (co-inheritance of physically adjacent genes), described in e.g. Egeland, J. A. et al. (1987) “Bipolar affective disorders linked to DNA markers on chromosome 11.” Nature, 325:783-787). Alternatively, polynucleotide sequences can be used as probes in Southern Blot analysis to identify alterations in the organization of the gene of interest and surrounding regions. Ultimately, complete sequencing of genes from several individuals can be performed to confirm the presence of a mutation and to distinguish mutations from polymorphisms. If a specific mutation is observed in some or all individuals affected by a particular disease, but not in any unaffected individuals, then the mutation is likely to be the causative agent of the particular disease

2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. The sequences of the present invention are useful as additional DNA markers for RFLP mapping (described in U.S. Pat. No. 5,272,057).

Furthermore the sequences of the present invention can be used to determine the actual base-by-base DNA sequence of selected portions of an individual's genome. Thus, the nucleic acid sequences described herein can be used to prepare two PCR primers from the 5′ and 3′ ends of the individual's DNA and subsequently sequence it. Panels of corresponding DNA sequences from individuals, prepared in this manner, can provide unique individual identifications, as each individual will have a unique set of such DNA sequences due to allelic variation. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the non-coding regions. It is estimated that allelic variation between individual humans occurs with a frequency of about once per 500 bases. Thus, each of the sequences described herein may be, to some degree, used as a standard against which DNA from an individual can be compared for identification purposes.

3. Forensic Biology

In addition the sequences described herein can be used in forensic biology. Forensic biology is a scientific field employing genetic typing of biological evidence found at a crime scene as a means for positively identifying, for example a perpetrator of a crime. To make such an identification, PCR-based technology can be used to amplify DNA sequences taken from very small biological samples such as tissues, (e.g. hair, skin, or body fluids). The amplified sequence can then be compared to a standard thereby allowing identification of the origin of the biological sample.

The sequences of the present invention can be used to provide polynucleotide reagents (e.g. PCR primers) targeted to specific loci in the human genome, which can enhance the reliability of DNA-based forensic identifications by, for example, providing another “identification marker” (i.e. another DNA sequence that is unique to a particular individual. The nucleic acid sequences described herein can further be used to provide polynucleotide reagents e.g. labeled or labelable probes, which can be used in, for example, an in situ hybridization technique, to identify a specific tissue. This technique can be exceedingly useful in cases where a forensic pathologist is presented with a tissue of unknown origin. Panels of such probes can be used to identify tissue by species and/or organ type.

Q. Predictive Medicine

Portions or fragments of the polynucleotide sequences of the invention can be used for predictive purposes to thereby treat an individual prophylactically.

1. Diagnostic/Prognostic Assays

One method of detecting the presence or absence of a polypeptide or nucleic acid in a biological sample is to expose that sample to an agent that recognizes the entity in question. A preferred agent for detecting mRNA or genomic DNA is a labeled nucleic acid probe capable of hybridizing to the sequence one is attempting to detect (for instance, the sequence of the invention). The nucleic acid probe can be, for example, a full length cDNA, or a portion thereof such as an oligonucleotide of at least 15, 30, 50, 100, 250, or 500 nucleotides in length and sufficient to specifically hybridize under stringent conditions to a mRNA or genomic DNA encoding the invention. The term “labeled” in this context refers to modifications in said sequences including, but not limited to, biotin labeling that can then be detected with a fluorescently labeled streptavidin, or ³²P labeling.

A preferred agent for detecting a polypeptide of the invention is an antibody or peptide capable of binding to the invention, preferably an antibody with a detectable label. Antibodies can be polyclonal or more preferably, monoclonal. An intact antibody, or a fragment thereof (e.g. a Fab or F(ab)₂) can be used. The term “labeled” in this context refers to direct labeling of the probe or antibody by coupling (i.e. physical linking) a detectable substance to the probe or antibody, such as a fluorescent labeled moiety or biotin.

The detection methods of the invention can be used to detect mRNA, protein, or genomic DNA in a biological sample in vitro as well as in vivo. For example, in vitro techniques for detection of mRNA include (but are not limited to) Northern Blot hybridization and in situ hybridizations. In vitro techniques for detection of a polypeptide of the invention include enzyme linked immunosorbent assays (ELISA's), Western blots, immunoprecipitations, and immunofluorescence.

The invention also encompasses kits for detecting the presence of a polypeptide or nucleic acid of the invention in a biological sample. Such kits can be used to determine if a subject is suffering from or is at increased risk of developing a disorder associate with aberrant expression of a polypeptide or polynucleotide of the invention. For instance, the kit can comprise a labeled compound or agent (as well as all the necessary supplementary agents needed for signal detection e.g. buffers, substrates, etc . . . ) capable of detecting the polypeptide, or mRNA in the sample (e.g. an antibody which binds the polypeptide or a oligonucleotide probe that binds to DNA or mRNA encoding the polypeptide).

The methods of the invention can also be used to detect genetic lesions or mutations in a gene of the invention, thereby determining if a subject with the lesioned gene is at risk for a disorder characterized by aberrant expression or activity of an agent of the invention. In preferred embodiments, the methods include detecting the presence or absence of a genetic lesion or mutation characterized by at least one alteration affecting the integrity of the agent of the invention. For example, such genetic lesions or mutations can be detected by ascertaining the existence of at least one of: 1) a deletion of one or more nucleotides from a gene; 2) an addition of one or more nucleotides to a gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA; 8) a non-wild type level of the protein encoded by the gene; 9) an allelic loss of the gene; and 10) an inappropriate post translational modification of the protein encoded by the gene. Many techniques can be used to detect lesions such as those described above. For instance, mutations in a selected gene from a sample can be identified by alterations in restriction enzyme cleavage patterns. In this procedure, sample and control DNA is isolated, digested with one or more restriction endonucleases, and fragment length sizes (determined by gel electrophoresis) are compared. Observable differences in fragment length sizes between sample and control DNA indicates mutations in the sample DNA. Additional techniques that can be applied to detecting mutations include, but are not limited to, detection based on direct sequencing, PCR-based detection of deletions, inversions, or translocations, detection based on mismatch cleavage reactions (Myers, R. M. et al. (1985) “Detection of single base substitutions by ribonuclease cleavage at mismatches in RNA:DNA duplexes.” Science 230:1242), and detection based on altered electrophoretic mobility (e.g. SSCP, see, for example, Orita, M. et al. (1989) “Detection of polymorphisms of human DNA by gel electrophoresis as single-strand conformation polymorphisms.” PNAS 86:2766).

2. Pharmacogenetics

Pharmacogenetics deals with clinically significant hereditary variation in the response to drugs due to altered drug disposition and altered action in affected persons (see Linder, M. W. et al. (1997) “Pharmacogenetics: a laboratory tool for optimizing therapeutic efficiency.” Clin Chem. 43(2):254-266). In general, two types of pharmacogenetic conditions can be differentiated. There are genetic conditions transmitted as a single factor altering the way drugs act on the body, referred to as “altered drug action”. Alternatively, there are genetic conditions transmitted as single factors altering the way the body acts on drugs (referred to as “altered drug metabolism”). These two conditions can occur either as rare defects, or as polymorphisms. For example, glucose-6-phosphate dehydrogenase deficiency is a common inherited enyzmopathy in which the main clinical complication is haemolysis after ingestion of oxidant drugs (e.g. anti-malarials, sulfonamides etc.).

The activity of drug metabolizing enzymes is a major determinant of both the intensity and duration of drug action. The discovery of genetic polymorphisms of drug metabolizing enzymes (e.g. N-acetyltransferase 2 (NAT2) and cytochrome P450 enzymes (CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM which all lead to the absence of functional CYP2D6. Poor metabolizers of this sort quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. At the other extreme are the so-called ultra rapid metabolizer who do not respond to standard doses. Recently, the molecular basis of ultra rapid metabolism has been identified to be due to CYP2D6 gene amplification.

Thus the in the context of pharmacogenetics, an agent of the invention can be used to determine or select appropriate agents for therapeutic prophylactic treatment of the individual. In addition, pharmacogenetic studies can be used to apply genotyping of polymorphic alleles encoding drug-metabolizing enzymes to the identification of an individuals drug responsiveness phenotype.

3. Monitoring of Effects During Clinical Trials

Monitoring the influence of agents that effect the expression or activity of a polypeptide or polynucleotide of the invention can be applied in clinical trials. For example, the effectiveness of a drug directed toward a target identified by the invention and intended to treat a particular ailment, can be monitored in clinical trials of subjects exhibiting said ailment by monitoring the level of gene expression of the target, activity of the target, or levels of the target of the invention. Thus in a preferred embodiment, the present invention provides a method for monitoring the effectiveness of treatment of a subject with an agent by comprising the steps of (i) obtaining a pre-administration sample from a subject prior to administration of the agent; (ii) detecting the level of the polypeptide or polynucleotide of the invention in the pre-administration sample; (iii) obtaining one or more post-administration samples from the subject; (iv) detecting the level or activity of said target of the invention in the post-administration samples, (v) comparing the level of said target of the invention in the post administration sample with levels in the pre-administration samples, and (vi) altering the administration of the agent to the subject accordingly.

R. Target Identification

After completion of the negative selection protocols as described herein, it is often advantageous to obtain the endogenous cellular protein(s) that promote the lethal phenotype. Such endogenous cellular proteins may serve a variety of functions in the cell, including without limitation (i) enzymatic function, (ii) protein-protein interaction in a pathway in the cell cytoplasm or nucleus; and (iii) transmembrane or secreted proteins, including signaling and transport proteins and the like.

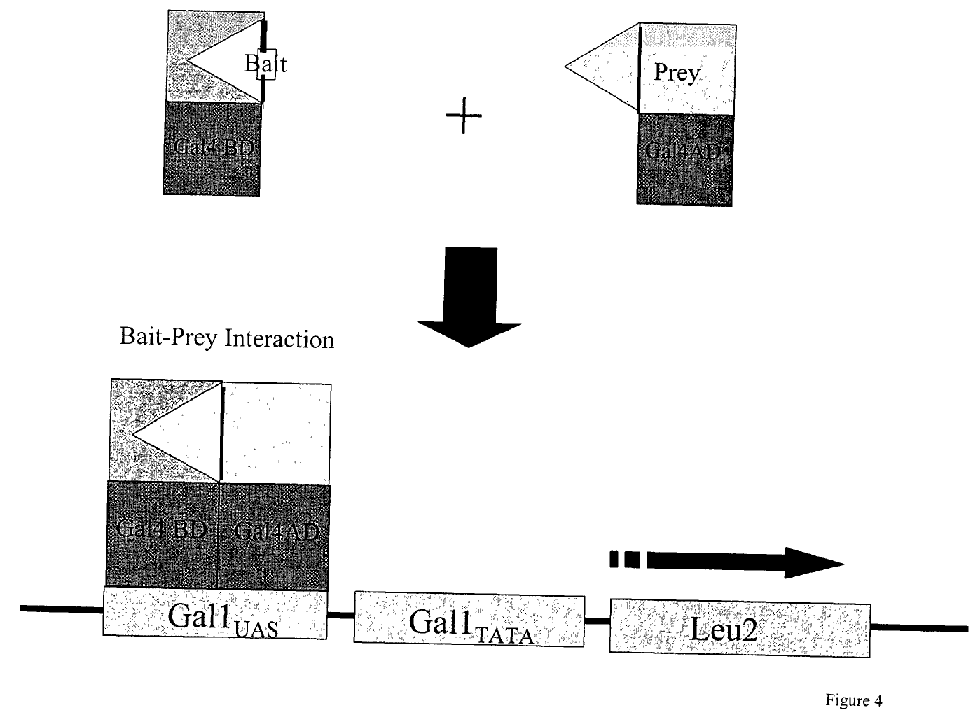

Targets of specific perturbagens may be identified by several means. For instance, peptide perturbagens can be modified with homo- or hetero-bifunctional coupling reagents and targets can be identified by chemical cross-linking techniques (see, for example, Tzeng, M. C. et al. (1995) “Binding proteins on synaptic membranes for crotoxin and taipoxin, two phospholipases A2 with neurotoxicity.” Toxicon. 33(4):451-7; Cochet, C. et al. (1988) “Demonstration of epidermal growth factor-induced receptor dimerization in living cells using a chemical covalent cross-linking agent.” J Biol Chem. 263(7):3290-5). Alternatively, one may use various techniques in column affinity chromatography or immunoprecipitation as a method of isolating and identifying target molecules (see, for example, Hentz, N. G. and Daunert, S. (1996) “Bifunctional fusion proteins of calmodulin and protein A as affinity ligands in protein purification and in the study of protein-protein interactions.” Anal Chem. 68(22): 3939-44). A preferred method involves application of a variation of the standard two-hybrid technology. See, e.g., U.S. Ser. No. 09/193,759 and WO 00/29565 “Methods for validating polypeptide targets that correlate to cellular phenotypes”, the entire disclosures of which are incorporated by reference herein. Generally stated, the two-hybrid procedure is a quasi-genetic approach to detecting binding events. This assay often is performed in yeast cells (although it can be adapted for use in mammalian and bacterial cells), and relies upon constructing two vectors; the first having an interaction probe or “bait” (that in this case, is the perturbagen) that typically is fused to a DNA binding domain (“BD”) moiety, and a second vector having an interaction target or “prey” (e.g. a cDNA library) that typically is fused to a DNA transcriptional moiety (the “activation domain” or “AD”). In an optimal setting, neither of the two fusion proteins can, individually, induce transcription of the reporter gene. Yet when the bait and prey interact, the AD and BD moieties are brought into sufficient physical proximity to result in transcription of a reporter gene (e.g., the Leu2 gene or lacZ gene) located downstream of the bound complex (FIG. 4). Prey/bait interactions are then detected by identifying yeast cells that are expressing the reporter gene—e.g. which express lacZ or are able to grow in the absence of leucine.

A variety of yeast host strains known in the art are suitable for use for identifying targets of individual perturbagens. One of ordinary skill will appreciate that a number of factors may be considered in selecting suitable host strains, including but not limited to (1) whether the host cells can be mated to cells of opposite mating type (i.e., they are haploid), and (2) whether the host cells contain chromosomally integrated reporter constructs that can be used for selections or screens (e.g., His3 and LacZ). Although mating can be desirable in some embodiments, it is not strictly necessary for purposes of practicing the present invention. For example, the mating procedures can be eliminated by introducing the bait and prey constructs into a single yeast cell, whereupon the screens can be performed on the haploid cell.

Generally, either Ga14 strains or LexA host strains may be used with the appropriate reporter constructs. Representative examples include strains yVT69, yVT87, yVT96, yVT97, yVT98 and yVT99, yVT100, yVT360. Additionally, those of ordinary skill will appreciate that the host strains used in the present invention may be modified in other ways known to the art in order to optimize assay performance. For example, it may be desirable to modify the strains so that they contain alternative or additional reporter genes that respond to two-hybrid interactions.

The following host yeast strains are thus constructed to have the indicated characteristics:

YVT69: yVT69 (mat □, ura3-52, his3-200, ade2-101, trp1-901, leu2-3, 112, gal4Δ, met⁻, gal80Δ, URA3::GAL1_{UAS}-GAL1_{TATA}-lacZ) was obtained from Clontech (Y187).

YVT87: yVT87 (Mat-α ura3-52,his3-200,trp1-901,LexAₒₚ₍ₓ₆₎-LEU2-3,112) was obtained from Clontech (EGY48).

YVT96: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT96, MATa ura3-52 his3-200 ade 2-101 ade5 lys2::GAL2-URA3 leu2-3, 112 trp1-901 tyr1-501 gal4D gal80Δ ade5::hisG by homologous recombination of Reporter 1 to the LYS2 locus. The integration is confirmed by PCR.

YVT97: The starting strain is YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 will be converted to yVT97, MATα ura3-52 his3::GAL1 or GAL7-HIS3 ade2-101 ade5 lys2-801 leu2-3, 112 trp1-901 tyr1-501 gal4Δ gal80Δ ade5::hisG by the steps of (a) converting from MATa to MATα via transient expression of the HO endonuclease, Methods in Enzymology Vol. 194:132-146 (1991) and (b) integrating either of Reporters 3 or 4 at the HIS3 locus via homologous recombination. The integration is confirmed by PCR.

YVT98: The starting strain was EGY48 (Estojak, J. Et al., 1995) MATα, ura3 his3 trp1 leu2::LexAop(x6)-LEU2. EGY48 was converted to strain yVT98 MATα ura3 his3 trp1 leu2::lexAop(x6)-LEU2 lys2::lexAop(8× or 2×)-LacZ by homologous recombination of Reporter 6 into the LYS2 locus.

YVT99: The starting strain was EGY48 (Estojak, J. Et al., 1995) MATα, ura3 his3 trp1 leu2::LexAop(x6)-LEU2. EGY48 was converted to strain yVT99 MATa ura3 his3 trp1 leu2::lexAop(x6)-LEU2 lys2::lexAop(8× or 2×)-URA3 by homologous recombination of Reporter 2 into the LYS2 locus and by switching the mating type from MATα to MATa via transient expression of the HO endonuclease.

YVT100: The starting strain was YM4271 (Liu, J. et al., 1993) MATa, ura3-52 his3-200 ade2-101 ade5 lys2-801 leu2-3, 112 trp1-901 tyr1-501 gal4Δ gal80Δ ade5::hisG. YM4271 was converted to yVT100, MATa ura3-52 his3-200 ade2-101 ade5 lys2::lexAop(8× or 2×)-URA3 leu2-3, 112 trp1-901 tyr-501 gal4Δ gal80Δ ade5::hisG by homologous recombination of Reporter 2 to the LYS2 locus. The integration was confirmed by PCR.

YVT360: yVT360 (mat a, tip1-901, leu2-3,112, ura3-52, his3-200, gal4Δ, gal 80Δ, LYS2::GAL1_{UAS}-GAL1_{TATA}-HIS³, GAL2_{UAS}-GAL2_{TATA}-ADE2, URA3:MEL1_{UAS}-MEL1_{TATA}-lacZ) was obtained from Clontech (AH109).

Exemplary yeast-reporter strains are constructed using a variety of standard techniques. Many of the starting yeast strains already carry multiple mutations that lead to an auxotrophic phenotype (e.g. ura3-52, ade2-101). When necessary, reporter constructs can be integrated into the genome of the appropriate strain by homologous recombination. Successful integration can be confirmed by PCR. Alternatively, reporters may be maintained in the cells episomally.

The yeast two-hybrid reporter gene typically is fused to an upstream promoter region that is recognized by the BD, and is selected to provide a marker that facilitates screening. Examples include the lacZ gene fused to the Gal1 promoter region and the His3 yeast gene fused to Gal1 promoter region. A variety of yeast two-hybrid reporter constructs are suitable for use in the present invention. One of ordinary skill will appreciate that a number of factors may be considered in selecting suitable reporters, including whether (1) the reporter construct provides a rigorous selection (i.e., yeast cells die in the absence of a protein-protein or peptide-protein interaction between the bait and prey sequences), and/or (2) the reporter construct provides a convenient screen (e.g., the cells turn color when they harbor bait and prey sequences that interact). Examples of desirable reporters include (1) the Ura3 gene, which confers growth in the absence of uracil and death in the presence of 5-fluoroorotic acid (5-FOA); (2) the His3 gene, which permits growth in the absence of histidine; (3) the LacZ gene, which is monitored by a colorimetric assay in the presence/absence of beta-galactosidase substrates (e.g. X-gal); (4) the Leu2 gene, which confers growth in the absence of leucine; and (5) the Lys2 gene, which confers growth in the absence of lysine or, in the alternative, death in the presence of α-aminoadipic acid. These reporter genes may be placed under the transcriptional control of any one of a number of suitable cis-regulatory elements, including for example the Gal2 promoter, the Gal1 promoter, the Gal7 promoter, or the LexA operator sequences.

The following are exemplary, non-limiting examples of such reporter constructs.

Reporter 1—(pVT85): This reporter comprises the URA3 gene under the transcriptional control of the yeast Gal2 upstream activating sequence (UAS). In order to facilitate integration of this reporter into the yeast chromosome in place of the Lys2 coding region, the Gal2-Ura3 construct is flanked on the 5′ side by the 500 base pairs that lie immediately upstream of the coding region of the LYS2 gene and on the 3′ side by the 500 base pairs that lie immediately 3′ of the coding region of the LYS2 gene. The entire vector is also cloned into the yeast centromere containing vector pRS413 (Sikorski, RS and Hieter, P., Genetics 122(1):19-27 (1989) and can therefore be used episomally. This reporter is intended for use with a Gal4-based two-hybrid system, e.g., Fields, S. and Song, O., Nature 340:245-246 (1989).

Reporter 2—(pVT86): This reporter is identical to reporter #1 except that the GAL2 UAS sequences have been replaced with regulatory promoter sequences that contain eight LexA operator sequences (Ebina et al., 1983). The number of LexA operator sequences in this reporter may either be increased or decreased in order to obtain the optimal level of transcriptional regulation. This reporter is intended to be used within the general confines of the LexA-based interaction trap devised by Brent and Ptashne.

Reporter 3—(pVT87): This reporter is comprised of the yeast His3 gene under the transcriptional control of the yeast Gal1 upstream activating sequence (UAS). In order to facilitate integration of this reporter into the yeast chromosome in place of the His3 coding region the Gal1-His3 construct is flanked on the 5′ side by the 500 base pairs (bp) immediately upstream of the His3 coding region and on the 3′ side by the 500 bp immediately 3′ of the His3 coding region. The entire reporter is also cloned into the yeast centromere containing vector pRS415 and can therefore be used episomally. This reporter is intended for use with a Gal4-based two-hybrid system.

Reporter 4—(pVT88): This reporter is identical to Reporter 3 except that the His3 gene is under the transcriptional control of Gal7 UAS sequences rather than the Gal1 UAS. The reporter is used with a Gal4-based two-hybrid system.

Reporter 5—(pVT89): This reporter contains the bacterial LacZ gene under the transcriptional control of the Gal1 UAS. The entire reporter will be cloned into a yeast centromere-using vector, e.g., pRS413, and is used episomally.

Reporter 6—(pVT90): This reporter consists of the LacZ gene under the transcriptional control of eight LexA operator sequences. As for Reporter 2, the number of LexA operator sequences in this reporter may either be increased or decreased in order to obtain optimal levels of transcriptional regulation. Two features of this reporter facilitate integration of the reporter into the yeast chromosome in place of the Lys2 coding region. First, it is flanked on the 5′ side by the 500 base pairs that lie immediately upstream of the coding region of the Lys2 gene and on the 3′ side by the 500 base pairs that lie immediately 3′ of the coding region of the Lys2 gene. Second, the neomycin (NEO) resistance gene has been inserted between the 5′ Lys2 sequences and the LexA promoter sequences. This reporter is used in conjunction with a LexA-based interaction trap, e.g., Golemis, E. A., et al., (1996), “Interaction trap/two hybrid system to identify interacting proteins.” Current Protocols in Molecular Biology, Ausebel et al., eds., New York, John Wiley & Sons, Chap. 20.1.1-20.1.28.

In other embodiments, perturbagen-induced phenotypes may be the result of RNA-polypeptide or polypeptide-DNA interactions. In cases such as these, variations of the original two-hybrid theme may be applied to identify the target of the phenotypic probe. (See, for example, Li, J. J. and Herskowitz, I. (1993) Isolation of Orc6, a Component of the Yeast Origin Recognition Complex by a One-Hybrid System. Science, 262:1870-1874; Svinarchuk, F. et al. (1997) “Recruitment of transcription factors to the target site by triplex-forming oligonucleotides.” NAR 25: 3459-3464; Segupta, D. J. et al. (1999) “Identification of RNAs that bind to a specific protein using the yeast three-hybrid system.” RNA 5:596-601; Harada, K. et al. (1996) “Selection of RNA-binding peptides in vivo.” Nature 14;380(6570):175-9; SenGupta, D. J. et al. (1996) “A three-hybrid system to detect RNA protein interactions in vivo.” PNAS 93:8496-8501). For instance, if evidence exists that a perturbagen is acting as an anti-sense agent, it is necessary to construct a system where the association of the DNA binding domains and the transcriptional activation domains is dependent upon and RNA-RNA interaction. To accomplish such a screen, four unique vectors are created. The first vector consists of the DNABP (e.g. GAL4 BD) described previously, linked to a specific RNA binding protein, arbitrarily called “RNABP-A” (e.g. the Rev responsive element RNA binding protein, RevM10, see Putz, U. et al. (1996) “A tri-hybrid system for the analysis and detection of RNA-protein interactions.” NAR 24:4838-4840). Vector #2 contains the transcriptional activation domain (e.g. GAL4 AD) linked to a second RNA binding protein (“RNABP-B”, e.g. the MS2 coat protein of the MS2 bacteriophage, see for example, SenGupta, D. J. et al. (1996) “A three hybrid system to detect RNA-protein interactions in vivo.” PNAS 93:8496-8501). The third vector encodes an RNA molecule that is recognized by RNABP-A (e.g. the RRE sequence, Zapp, M. L. and Green M. R/“Sequence-specific RNA binding by the HIV-I Rev protein (1989) Nature, 32:714-716) fused to a sequence encoding the RNA perturbagen, while the final vector encodes a fourth hybrid, the RNA sequence recognized by RNABP-B (e.g. the 21 base nucleotide RNA stem-loop structure of MS2, see Uhlenbeck, O. C. et. al. (1983) “Interaction of R17 coat protein with its RNA binding site for translational repression.” J Biomol Struct. Dyn. 1, 539-552) linked to a library of expressed sequences (e.g. a library of mRNA molecules). When all four vectors are stably maintained in a yeast cell containing the necessary reporter construct(s) (e.g. P_{GAL4}-LACZ), the cellular target RNA molecule of any given RNA perturbagen can be identified.

In some instances, a particular phenotype may be the result of a perturbagen differentially regulating a distinct combination of genes. For example, a perturbagen might, through its interaction with a particular transcription factor which, in turn, recognizes a particular DNA promoter sequence, elevate the expression of two or more target genes that act in concert to elicit a unique phenotype (e.g. cell death). In these cases, each of the genes whose levels of expression are altered by the perturbagen can be considered to be perturbagen targets and can be identified by a variety of techniques including (but not limited to) SAGE and expression profiling via microarray analysis (see, for instance, Cummings C. A. and Relman D. A. (2000) “Using DNA Microarrays to Study Host-Microbe Interactions.” Emerg Infect Dis. 6(5):513-525; Yamamoto M. et al. (2001) “Use of serial analysis of gene expression (SAGE) technology. J Immunol Methods. 2001 Apr;250(1-2):45-66).

Target sequences or fragments thereof can vary greatly in size. Some target fragments can be as small as ten amino acids in length. Alternatively, target sequences can be greater than 10 amino acids but less than thirty amino acids in length. Still other targets can be greater than thirty amino acids in length but shorter than 60 amino acids in length. Still other targets are cellular proteins or subunits or domains therein of more than 60 amino acids in length. Still other targets are cellular proteins or subunits or domains there of more than 60 amino acids in length. Still other targets are cellular proteins or subunits or domains there of more than 60 amino acids in length. In addition, for reasons described previously, the sequences encoding targets can vary greatly due to allelic variation, duplications and closely related gene family members. That said, the invention also encompasses variants of said targets. A preferred target variant is one which has at least about 80%, alternatively at least about 90%, and in another alternative at least about 95% amino acid sequence identity to the original target amino acid sequence and which contains at least one functional or structural characteristic of the original target

The following examples for the generation and use of the selection systems of the invention are given to enable those skilled in the art to more clearly understand and to practice the present invention. The present invention, however, is not limited in scope by the exemplified embodiments, which are intended as illustrations of single aspects of the invention only, and methods and materials that are functionally equivalent are within the scope of the invention. Various modifications of the invention in addition to those described herein will become apparent to those skilled in the art from the foregoing description and accompanying drawings. Such modifications are intended to fall within the scope of the appended claims.

### EXAMPLES

### Example One

### Methods for Identifying and Characterizing Dead and Dying Cells

Many negative selections, including some selections described herein, require the identification of dead and/or dying cells. As one of ordinary skill in the art appreciates, there are many techniques that can be used to detect these cells.

A number of techniques exist for identifying cells that have a lethal phenotype, and for distinguishing, e.g., apoptotic and necrotric cells. In some instances, antibodies are used to identify cells that are undergoing apoptosis. Koester et al., Monitoring Early Cellular Responses in Apoptosis is Aided by the Mitochondrial Membrane Protein-Specific Monoclonal Antibody APO2.7″ Cytometry, 29:306-312 (1997). In some such embodiments, the antibodies recognize antigens that are, under normal (viable) conditions, hidden or masked from detection, but which become exposed in dying cells. In other instances, apoptotic cells are detected using substrates that are recognized by proteases (caspases) that are unique to, and activated by, the apoptotic pathway. Green, D., Kroemer, G., “The Central Executioners of Apoptosis: Caspases or Mitochondria”. Trends in Cell Biology. 8:267-271 (1998). In still other instances, dead and dying cells are distinguished from viable cells on the basis of their interaction with various dyes. One class, membrane permeable dyes (e.g. Trypan Blue), are actively excluded from the intracellular compartments of living cells but accumulate in the cytoplasmic/nuclear regions of dead or dying cells. A second class of reagents, membrane impermeable dyes, is excluded from all living cells, but is capable of penetrating the compromised membrane boundaries of dead and/or dying cells. Many of these reagents (e.g. propidium iodide, ethidium homodimer) have an affinity for DNA and show an increase in fluorescence upon binding to nucleic acids. Krishan, “Rapid flow cytofluormetric analysis of mammalian cell cycle by propidium iodide staining,” J. Cell Biology 59:766 (1973). These reagents may be used in conjunction with FACS analysis or be applied in the more general techniques of fluorescent microscopy. Shapiro, “Practical Flow Cytometry”, H. M. Wiley-Liss Publications (1995).

A. Apo 2.7 Antibody Staining.

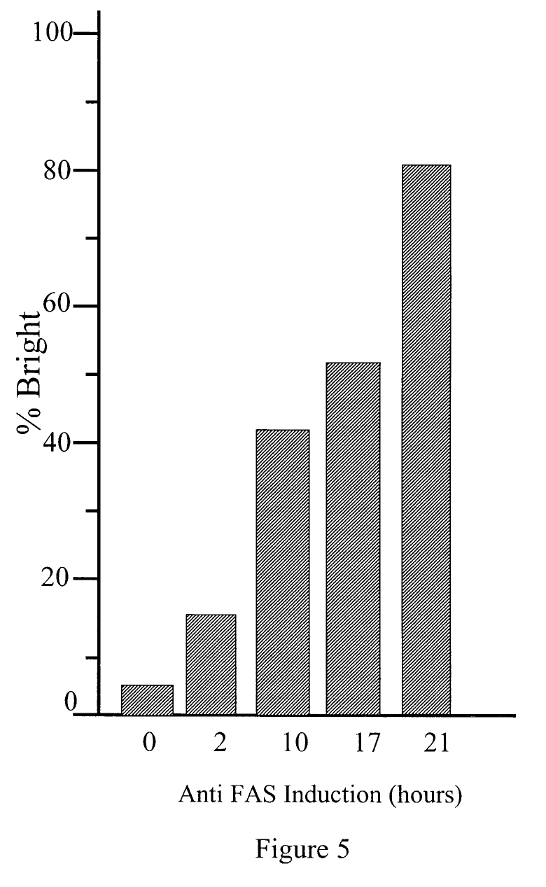

Apo2.7 (Coulter Immunotech) is a monoclonal antibody that recognizes an epitope in the mitochondrial membrane that is exposed only in cells that are undergoing apoptosis. To test its efficacy in negative selections, 1×10⁶ Jurkat cells in 2 milliliters of AIM-V serum free medium were induced to undergo apoptosis using the anti-FAS antibody (1 ug/ml anti CD95 clone, Yonehara, S. et al., “A cell killing monoclonal antibody (anti-FAS) to a cell surface antigen co-down regulated with the receptor of tumor necrosis factor.” J. Exp. Med 169: 1747-1756 (1989)). After a fixed period of exposure (0, 2, 10, 17, or 21 hours in anti-FAS antibody), 100 μl of a 100 μg/ml solution of digitonin in PBS was added (20 minutes on ice) to permeablize the cell membrane. Following this procedure, the cells were spun (200× g) and resuspended in a solution containing a fluorescent R-phycoerythrin-cyanin labeled Apo2.7 antibody provided by Coulter-Immunotech (10 μl Apo2.7 antibody, 90 μl PBS,+cells). This reaction was allowed to incubate for 15 minutes at room temperature in the dark. The cells were then pelleted by centrifugation (200× g) and resuspended in 1 ml of PBS before being analyzed by flow cytometry (excitation, 488 nm; emission, 660-690 nm). Results of FACS analysis (FIG. 5) showed that at time=0 (i.e. control), only 4% of the population labeled with Apo2.7 antibody. In contrast, exposure to the apoptotic-inducing anti-FAS antibody led to increased binding of Apo2.7 to the Jurkat cell line (t=21 hrs =81% labeling).

B. Propidium Iodide Staining.

Propidium iodide (PI) is a fluorescent, DNA intercalating, molecule. Live cells with intact membranes exclude PI from intracellular compartments and thus are non-fluorescent. Dead and dying cells whose membranes have been compromised are permeable to PI, and thus are fluorescent. The PI staining technique is equally applicable to apoptotic and necrotic cells.

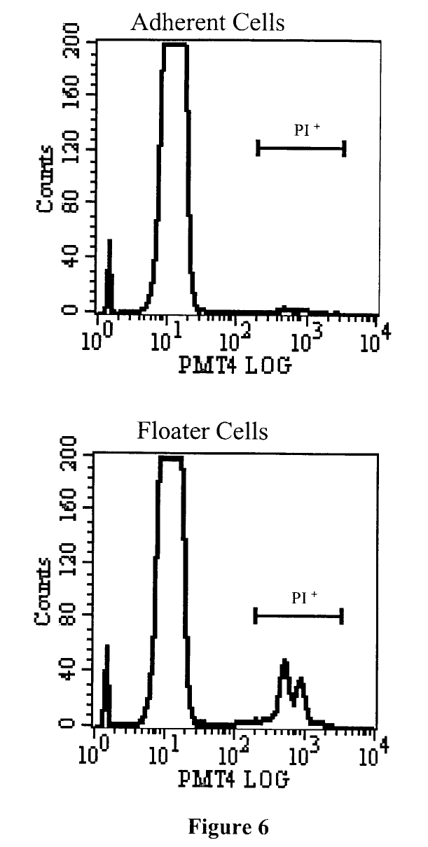

As a non-limiting example of the use of this type of reagent for identification and purification of dead cells, the following pilot experiment was performed. Floater cells (i.e., non-adherent cells in an otherwise adherent cell population) were isolated from a flask of HT-29 colon cancer cells. These cells, along with an equally sized adherent cell population, were harvested by centrifugation and subsequently resuspended at 1×10⁶ cells/ml in PBS. PI was then added to each sample at a concentration of 2.0 μg/ml and cells were analyzed by flow cytometry (excitation 488 nm, emission, 610 mn). Dead cells that had lost membrane integrity could be easily distinguished from live cells based on their increased fluorescence (FIG. 6). The percentage of adherent cells that were positive for PI uptake averaged approximately 0.5-1.5% at this cell density. In non-adherent “floater” cells, a higher percentage of cells (>10%) were observed to be positive for PI uptake.

To make a positive correlation between PI staining and cell viability, an equivalent number of PI positive and PI negative cells were identified and collected separately using FACS. These two populations were then plated onto 150 mm plastic tissue culture dishes and allowed to attach and grow for 7-10 days. Cell viability was then determined by counting the number of colonies that grew on each plate. While cells that were PI negative were viable and produced colonies, PI positive cells failed to grow.

C. Nuclear Condensation.

In contrast to necrosis, cells that die by apoptosis often exhibit nuclear condensation. Thus, dye/stain techniques that allow visualization of the nuclear morphology are used to assess the method by which a cell dies.

Two cell lines, WM35 and HS294T, were plated out (50,000 cells per well, 24 well plate) and allowed to adhere. After 24 hours, the cells were treated for 4 hours with varying concentrations (5-80 μM) of Cisplatin (cis-platinum (II) diammine dichloride), a well-known chemotherapeutic agent that induces apoptosis. The following day (18-24 hrs later) the media was then collected from each well and cells that did not adhere to the well (termed herein, “floaters cells” or “floaters”) were collected by centrifugation (400× g). Adherent cell populations were then lifted from the solid support by trypsinization, centrifuged (400× g), and resuspended in PBS (0.125 ml). To observe the nuclear morphology and percent cell death in the WM35 and HS294T cell lines, cells from both floater and adherent populations were stained concurrently with Syto16 and ethidium homodimer (125 ul of cell suspension+2.5 ul 62.5uM Sytol6+2.5uM 10 ug/ml ethidium homodimer, 10 minutes at 37° C.). Ethidium homodimer is a membrane impermeant compound that fluoresces in the 617 nm range when it is intercalated with chromosomal DNA. Thus, in a mixed cell population containing both living and dead/dying cells, only those cells whose membranes have been compromised will stain with ethidium homodimer. In contrast, Syto 16 (Molecular Probes) is a membrane permeant dye that fluoresces in the 518 nm range when associated with chromosomal DNA. Together, these two dyes can be used to observe and distinguish the nuclear morphology in a population containing both living and dead/dying cells.

Examination of the floater population of Cisplatin treated HS294T cells showed that while greater than 50% of the cells stained with ethidium homodimer, in general, fewer than 20% of these cells showed condensed or fragmented nuclei when observed by fluorescent microscopy. Instead, the nuclei in these cells appeared diffuse and bloated, suggesting that Cisplatin treated HS294T cells die by a necrotic, rather than an apoptotic, pathway. In contrast, the floaters obtained from Cisplatin treated WM35 cell lines showed both a high degree of ethidium homodimer staining (45-50%) and a phenotypically distinct condensed or fragmented nuclei (40-50% in higher concentrations of Cisplatin) suggesting that a large percentage of these cells die by an apoptotic pathway. Neither of the two adherent cell populations exhibited significant amounts of ethidium homodimer staining (generally <10%), indicating that the adherent population largely comprised viable cells.

D. Caspase-Sensitive Dyes.

Caspase-3 and other proteases have been shown to play a role in apoptotic induced cell death (Green and Kroemer, 1998). To test the correlation between this enzymes activity and cell death, and to study the possibility of using caspase-3 activity in negative selections, WM35 (melanoma) cells are induced to undergo apoptosis and then exposed to Rhodamine 123-YVAD, a caspase-3 fluorescent substrate.

WM35 cells were passed one day prior to induction of apoptosis and incubated for 24 hrs to allow the cells to attach to the substrate. The media was subsequently removed, the remaining adherent cells washed 1× with PBS, and subsequently exposed to Cisplatin (15 μg/ml) in fresh media. Eighteen to twenty-four hours after the induction of apoptosis, the floater cell population was collected, pelleted by centrifugation (400× g), and resuspended at 3×10⁶ cells/ml in PBS. Samples were then split into four groups: (I) uninduced minus Rhodamine 123-YVAD (substrate), (2) uninduced plus substrate, (3) induced minus substrate, and (4) induced plus substrate. For samples exposed to Rhodamine 123-YVAD substrate, 50 ul of a pre-warmed (37° C.) cell suspension was combined with 25 ul of a stock substrate solution (Celiprobe). Samples were incubated for 60 minutes at 37° C. and then placed on ice prior to FACS analysis. In addition to caspase-3 staining, a replicate of each sample was stained with propidium iodide to determine the percentage of cells within the population whose membranes had been compromised and the overlap between PI and caspase-3 staining. For flow cytometric analysis, each sample was brought to a total volume of 1 ml (PBS) and excited at a wavelength of 488 nm (15 mwatts) using an argon laser. Emission spectra were read at 515-535 nm wavelength using the FLI (PMT2) 525 nm blue filter.

Caspase-3 activity peaks early in the apoptotic cycle, long before the disruption of the cell cytoplasmic membrane. Therefore, caspase3-positive floater cells are predicted to be PI-negative, while PI-positive floater cells are expected to have passed the peak period of caspase-3 activity and therefore be phenotypically caspase-3-negative. Consistent with these predictions, of the PI-minus, Cisplatin-treated WM35 cells collected from the floater population, 94.6% were found to be caspase-3 positive. The remaining cells obtained from the floaters fell into the PI-positive, caspase-3-negative group. Control studies with adherent cell populations showed the vast majority (>95%) to be both caspase and PI negative.

### Example Two

### Identifying Cell Types for Negative Selections Via Floater Assays

Prior to performing negative selection assays, a cell line with a phenotypic feature that is a readily monitored surrogate for a lethal phenotype is identified. In this Example, lack of cellular adhesion to a plastic, gelatin or other suitable culturing support (i.e. presence of “floating cells” or “floaters”) is selected as the surrogate phenotypic feature that correlates to the lethal phenotype.

In order to use floater populations as a method of identifying and enriching for dead and/or dying cells in a negative selections, cell lines preferably display three features: (1) in a stable untreated cell population, the greater majority of cells are adherent to the solid support (e.g. plastic, gelatin) and the background rate of floater cells is relatively low (<1%); (2) in an untreated or treated cell population (i.e. one exposed to putative cytotoxic agents and optionally also a secondary agent), a high percentage of the floater cells correlate with the dead and/or dying cell population; and (3) the cell line is receptive to standard or common techniques of introducing library inserts encoding putative cytotoxic agents into the cell e.g. retroviral infection or transduction.

A. Background Levels of Floater Cells.

The first variable, background floater levels, are evaluated by establishing a stable culture of target cells and then comparing the levels of cells floating in the media with the total number of cells (adherent cells+floater cells). Additional procedures, such as retroviral infection, can be overlaid on top of this experimental design, thus making it possible to assess the effects of retroviral infection on floater cell/total cell ratios and determination of the receptiveness of the cell line to the introduction of putative cytotoxic agents by transfection.

HT29 cells were tested for feasibility in the floater cell assay as follows. Briefly, six flasks were inoculated with 6.25×10⁵ HT29 cells/flask on Day 0. On Day 1, after the cells had been allowed to adhere to the solid support, two of these flasks were infected with retroviral supernatant containing the retroviral vector pVT324, which includes a selectable drug resistant marker (e.g. neomycin), and which constitutively expresses a green fluorescent protein. Of the remaining four flasks, two were mock infected (i.e. exposed to all of the same reagents/conditions as flasks 1 and 2, minus the retrovirus, see “Example 3C”) and two were left undisturbed. On Day 2, this procedure was repeated (i.e. a double infection). On Day 3 (and subsequently on Day 5) one flask was selected from each of the three groups and processed by separating and counting the floater and adherent cell populations. In the case of the floater cell population, the media was collected, centrifuged at 200× g for 10 minutes, and resuspended in PBS prior to removing a sample for counting on a hemocytometer. For the adherent cell population, cells were first removed from the flask by trypsinization, centrifuged, and then processed for analysis in a fashion analogous to the floater cells. To determine the inherent background level of floater cells present in the population, the ratio of the number of floater cells to the total number of cells (adherent cells+floater cells) was analyzed on both the Day 3 and Day 5 flasks that had not been manipulated. These numbers were compared with the analogous numbers taken from infected and mock-infected flasks to determine whether the retrovirus or transfection procedures altered background floater rates. To determine the susceptibility of HT29 cells to retroviral infection, the fraction of cells that expressed GFP in pVT324 infected flasks was calculated using flow cytometry.

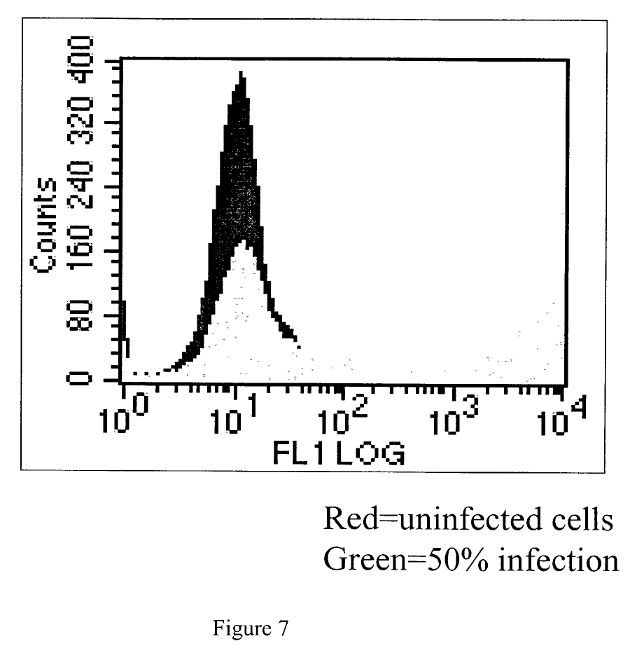

The non-infected background floater rate of the HT29 cell line was found to be 0.42%. Infected and mock infected HT29 cells showed 0.51 and 0.37% floater rates respectively, indicating that neither the retroviral infection procedures nor the retrovirus itself increases background floater rates substantially. In addition, FACS analysis of the pVT324 infected HT29 population showed approximately 80% of the cells falling into the “bright” gate (i.e. GFP expressing cells, FIG. 7). Together, the low background floater rate and the high susceptibility of HT29 to retroviral infection make it a desirable candidate for negative selections. Additional cells lines—two colorectal adenocarcinoma lines, SW620 and DLD-1 (CCL-221, ATCC), and a prostate adenocarcinoma cell line, PC-3—also were examined using these same criteria and been found to be suitable cell line candidates for negative selections. In contrast, LNCaP, a human prostate carcinoma cell line (ATCC), exhibited background floater rates of greater than 10% thus making it less preferred for use in negative selections which utilize lack of adhesion as a surrogate for a lethal phenotype. See Table 1, below.

TABLE 1

| | Colon | Colon | | Prostate | Prostate |
|---|---|---|---|---|---|
| | HT29 | SW620 | DLD-1 | PC-3 | LNCaP |
| Background | ≦1.0% | ≦1.0% | ≦1.0% | ≦1.0% | 4-5% |
| Death | | | | | |
| Floaters | ≦0.5% | 1.0% | 0.5% | 2.5% | 12% |
| Fraction of dead cells in | 40% | 65% | 50% | 30% | 12% |
| floaters | | | | | |
| Do dead cells eventually | YES | YES | YES | YES | ? |
| become floaters | | | | | |
| Tolerates retroviral | YES | YES | YES | YES | YES |
| infection | | | | | |

B. Correlation Between “Floaters” and Lethal Phenotypes.

An important component to the negative selections of the present invention is the ability to demonstrate a correlation between the floater population and dead and/or dying cells. This correlation can be established using a variety of techniques known to those of skill in the art, including without limit those described above for detecting and characterizing such cells.

In this Example, propidium iodide (PI) was used as a method of monitoring the percent dead/dying cells in both the adherent and floating cell populations. To determine the percent of floaters that were dead and/or dying, four separate cell lines (three colon cancer cell lines, HT29, SW620, and DLD-1, and one prostrate cancer cell line, PC3) were plated in tissue culture flasks and allowed to adhere. After 24-48 hours, both adherent cells and floaters were collected, stained with PI, and examined by flow cytometry. While the adherent populations of all four cell lines typically showed less than 1% PI⁺ cells, 30% or more of the floater cell population were observed to be PI⁺.

These studies demonstrate that there is a strong correlation between cell death and floaters in the above cell lines. In addition, combining the techniques of floater collection with a second selection (FACS sorting of PI⁺ cells) enables one to further maximize the level of enrichment of dead and/or dying cells having a lethal phenotype and thus, increasing the likelihood of isolating cytostatic agents that exist at low frequency in the population.

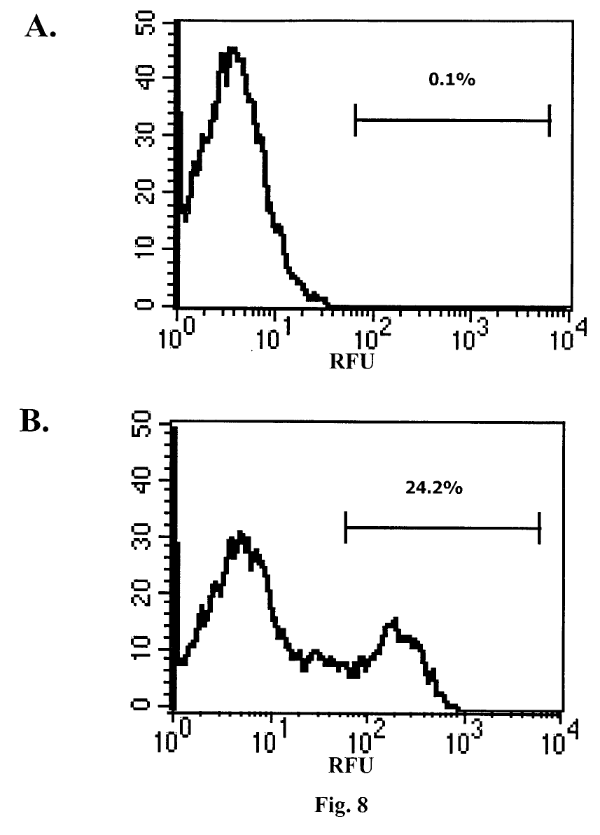

A second experiment designed to determine whether dead or dying cells move from the adherent to floater population was performed using a pulse-chase protocol. Alberts, B. et al. “Molecular Biology of the Cell”, pg. 180, Garland Publishing, Inc. (1983). Adherent PC-3 cells in culture were stained for a brief period with 2 μg/ml PI while remaining attached to the plate. The cells were then rinsed and returned to fresh media. Twenty-four hours later, both the floater and adherent populations were collected and scanned to determine the distribution of PI positive cells amongst the two groups of cells. The results (FIG. 8) show that the PI positive cells segregate specifically to the non-adherent “floater” population of cells. This result indicates that dead or dying cells that had previously been adherent move into the floater population within 24 hours.

### Example Three

### Introduction and Recovery of Sequences Encoding Cytotoxic Agents

In order to perform “floater assays” to identify sequences that encode cytotoxic agents (i.e., agents that stimulate relatively immediate death of individual cells, or agents that prevent cell growth or proliferation, thus gradually leading to the death of a cell population), libraries of sequences encoding putative cytotoxic/cytostatic agents are constructed and then introduced into the selected cell lines. The following Example describes one non-limiting protocol for such work.

A. Preparation and Transfer of a cDNA Library

Using techniques that are common to individuals familiar with the art, polyA mRNA is isolated from fetal brain tissue by affinity chromatography on an oligo dT cellulose column (polyASpin™, New England BioLabs). This material is then subjected to first strand PCR (Pfu polymerase, Stratagene) synthesis using oligo dT primers linked to sequences encoding a selected restriction enzyme linker. Following the elimination of RNA (RNAse A/H, Boehinger Mannheim) from the sample, second strand synthesis proceeds, using random primed oligos that have been constructed with the desired linker sequence. The double stranded cDNA product is then size selected, treated with the appropriate enzymes to create “sticky” ends, and ligated into an expression vector suitable for the cell line of choice.

As an alternative to oligo dT primed cDNA libraries, randomly primed cDNA libraries are used as a source of sequences encoding putative cytotoxic agents. As one non-limiting example of how to construct such a library, polyA mRNA derived from placental tissue was PCR amplified using a random 9-mer linked to a unique SfiI sequence (“SfiA”), followed by an additional set of nucleotides that is used later for library amplification (OVT 906:5′ ACTCTGGACTAGGCAGGTTCAGTGGCCA TTATGGCC(N)₉). The product of this reaction was size selected (>400 base pairs) and subjected to RNAseA/H treatment to remove the original RNA template. The remaining single stranded DNA was then subjected to a second round of PCR using a random hexamer nucleotide sequence linked to a second unique SfiI sequence (“SfiB”) which was again followed by an additional set of nucleotides for future library amplification: (OVT 908:5′ AAGCAGTGGTGTCAACGCAG TGAGGCCGAGGCGGCC(N)₆). The final product of this reaction was blunted/filled with Klenow Fragment (New England BioLabs), size selected, PCR amplified (OVT 909:5′ ACTCT GGACTAGGCAGGTTCAGT and OVT 910:5′ AAGCAGTGGTGTCAACGCAG TGA), digested with SfiI (New England BioLabs), and inserted into a retroviral vector.

Alternatively, commercially available libraries can be used. The cDNA inserts of such libraries are spliced out from the original vector and inserted into an expression vector of choice. As one non-limiting example, three libraries obtained from three different tissue sources (brain, liver, and kidney) were obtained from Origene Inc. (Catalogue # DHL101, DHL 105, and DHL 106). Using standard techniques, bacterial hosts carrying the libraries were expanded in liquid media (LB plus ampicillin) and used to prepare large quantities of episomal (library) DNA (Maxiprep, Qiagen). The cDNA insert in each vector was then released by digestion with the appropriate restriction enzyme (EcoRI/XhoI) and the fragments were then gel purified (0.4-2.8 kB) and ligated (T4 Ligase, Boehringer Mannheim) into the compatible sites of the pVT340 retroviral vector (described below).

B. Construction of A Scaffolded Peptide Library

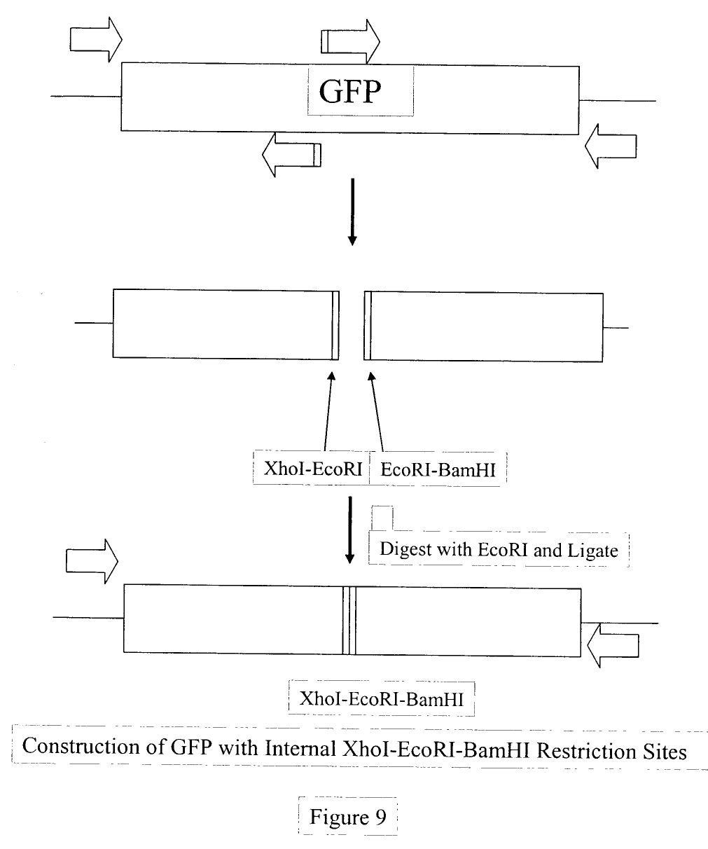

Construction of a scaffolded peptide library followed the protocols developed by Abedi et al., N.A.R. 26(2): 623-630 (1998), incorporated by reference herein in its entirety. Initially a modified GFP containing BamHI, XhoI, and EcoRI sites at position 6 (pVT27) was constructed using pVTO14 (also known as pACA151, a gift of Dr. Jasper Rine) as a template. To accomplish this, two separate PCR reactions using oligos OVT 312 (5′ TGAGAA TTCCTCGAGTTGTTTGTCTGCCATGATGTATAC), OVT 322 (5′ TGAGAATTCG GATCCAAGAATGGAATCAAAGTTAACTTC), OVT 329 (5′ GTTAGCTCACTCA TTAGGCACCC) and OVT 330 (5′ CGGTATAGATCTGTATAGTTCATCC ATGCCATGTG) were performed using recombinant Pfu polymerase (Stratagene). The internal termini of the resulting fragments contained XhoI/EcoRI and EcoRI/BamHI restriction sites (FIG. 9). The two fragments were subsequently digested with EcoRI (New England Biolabs), ligated with T4 DNA Ligase (Boehringer Mannheim) and PCR amplified using the external primers OVT 329 and OVT330. The final product contains a 6 codon insert incorporating XhoI/EcoRI/BamHI restriction sites at the Gln157-Lys 158 insertion site of pVT27.

To construct the random peptide library, fifteen picomoles of Aptamer 3 (5′ TCGAGA GTGCAGGT[NN(G/C/T)]₁₅GGAGCTTCTG) was mixed with Aptamer 4 (5′ ACCTGC ACTC) and Aptamer 5 (5′ GATCCAGAAGCTCC) in a molar ratio of 1:50:50 and annealed in 20 mM Tris-HCl, pH 7.5, 2 mM MgCl₂, 50 mM NaCl by heating to 70° C. for 5 minutes. The solution was then allowed to cool to room temperature and ligated to a BamHI/XhoI cut pVT 334 retroviral vector using T4 ligase (Boehringer Mannheim). As a result of these manipulations, a biased-random fifteen amino acid sequence flanked by three constant amino acids on either end was inserted into position 6/VT27 of GFP. The library was transformed into E. coli (DH10B, Gibco) by electroporation and plated on LB-agar plates containing the selective drug, ampicillin.

C. Expression Vectors

A variety of retroviral or other vectors are suitable for use in the invention. As one non-limiting example, of a retroviral expression vector useful for constitutive expression of library sequences in mammalian cells was constructed as follows. The 3.8 kB HindIII/ScaI band of pVT314 was ligated to the 1.9 kB SSPI/PvuII band of pBluescript™ (Stratagene). The final product of this reaction (referred to as pCLMFG, or MFG or pVT340) is a vector that contains all the necessary components of a constitutive retroviral expression vector including a Psi site for packaging, constitutive CMV driven expression, a splice donor and acceptor site for obtaining high levels of library insert expression, and a multiple cloning site (MSC) linked to the 3′ end of EGFP. Putative cytotoxic agents are expressed constitutively as fusions with the GFP scaffold.

As an alternative to the constitutive pCLMFG vector, an inducible construct that can be regulated by ecdysone was constructed as follows. The PmlI/XhoI fragment from pVT324 was inserted into the MCS of the PIND vector (Invitrogen). This product was then digested with BglII, blunt-ended and inserted into a pBabe-K-ras vector (pVT313-based) that had been digested with BamHI/XhoI and blunted (Klenow Fragment). The resulting vector was designated pBabe-Forward-1. The XbaI fragment of pVT324 was then inserted into the compatible site of pBabe-Forward-1. The resulting vector was designated pBIGFII. Vector pBIGFII was subsequently transfected into cells (ECR293, Invitrogen) that contain an endogenous copy of the ecdysone receptor (pVgRXR). When these cells are grown in the absence of ponesterone A, they exhibit a low level of background fluorescence. In contrast, when the cells containing both vectors are grown in the presence of 5 μM ponesterone A, the level of fluorescence increases by approximately thirty fold. Thus, pBIGFII exhibits a low background fluorescence and is strongly induced in the presence of ponesterone A. Such vectors are useful in identifying sequences encoding cytotoxic agents that disrupt the cell cycle or induce death via an apoptotic pathway.

D. Retroviral Packaging and Infection

Next, the library constructs are packaged for retroviral transfection into the cell of choice. One non-limiting method of accomplishing this is described as follows. On Day 1, 3×10⁶ cells of the packaging cell line (293gp) are seeded into a T175 flask. On the second day, two tubes, one carrying 15 ug of library DNA+10 ug of envelope plasmid (pCMV-VSV.G-bpa)+1.5 ml DMEM (serum free), the second carrying 100 ul of LipofectAMINE (Gibco BRL)+1.5 ml DMEM (serum free) are mixed and left at room temperature for 30 minutes. Subsequently, the two tubes are mixed together along with 17 ml of serum free DMEM. This cocktail is referred to as the “transfection mix.” Previously plated 293gp cells are then gently washed with serum free media and exposed to 20 ml of the transfection mix for 4 hours at 37° C. Following this period, the transfection mix can be removed and the cells are incubated with complete DMEM (10% serum) for a period of 72 hours at 37° C. On Day 4 or 5, the media (now referred to as “viral supernatant”) overlying the 293gp cells is collected, filtered through a 0.45 μ filter and frozen down in at −80° C.

As an alternative to the LipofectAMINE method of retroviral DNA packaging, a second protocol, referred to herein as the “CaCl₂ Method,” can be used to package retroviral sequences. In this method, 5×10⁶ cells of the packaging cell line (293gp) are seeded into a 15 cm² flask on Day 1. On the following day, the media is replaced with 22.5 mls of modified DMEM. Subsequently, a single tube carrying 22.5 μg of retroviral library DNA and 22.5 μg of envelope expression plasmid (pCMV-VSV.G-bpa) is brought to 400 μl with dH₂O, to which is added 100 μl of CaCl₂ (2.5M) and 500 μl of BBS (dropwise addition, 2× solution=50 mM, BES (N,N-bis(2-hydroxyethyl)-2-aminoethane-sulfonic acid), 280 mM NaCl, 1.5 mM Na₂HPO₄, pH 6.95). After allowing this retroviral mixture to sit at room temperature for 5-10 minutes, i.e. is added to the 293gp cells in a dropwise fashion, and the cells are then incubated at 37° C. (3% CO₂) for 16-24 hours. The media is then replaced and the cells are allowed to incubate for an additional 48-72 hours at 37° C. At that time, the media containing the viral particles is then collected, filtered through a 0.45% filter and frozen down at −80° C.

To infect the cell line or primary cells of interest, the selected target cells (e.g. HT29, SW620) are plated out at a density of approximately 1.5×10⁶ cells per T175 flask. On the following day (Day 1), the library supernatant is added directly to the media (10-30% total volume) along with 4 μg/ml polybrene and allowed to incubate overnight. On Days 2 and Day 3, the supernatant is removed and replaced with fresh media. Floater cell populations are then collected on Days 3-5.

E. Recovery of Cytotoxic Sequences from Dead and/or Dying Cells.

In order to identify cytotoxic agents or substances which cause cell death, those agents (or the DNA sequences that encode them) are recovered from dead and/or dying cells.

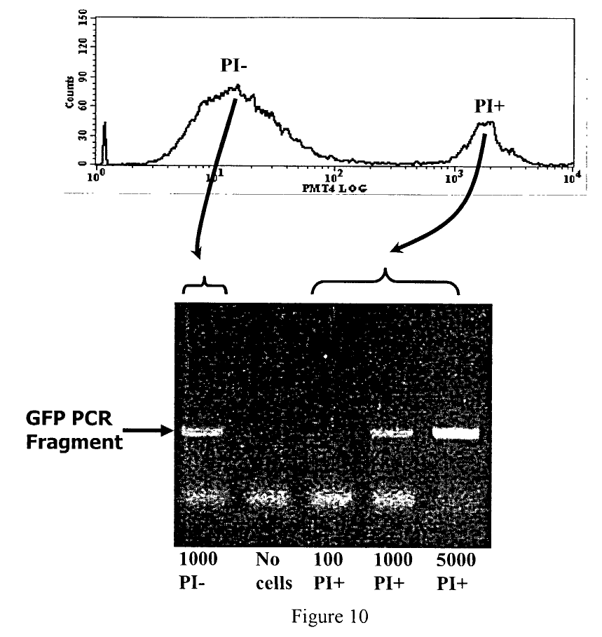

Briefly, PCR is used to rescue and amplify DNA sequences encoding cytotoxic agents from non-viable cells. McPherson, M. J. et al., “PCR 2. A practical approach.” Oxford University Press (1995). To compare the sensitivity of PCR on dead cells with that of viable cells, HT29 cells carrying a constitutive GFP encoding retroviral insert (pVT324) were induced to undergo apoptosis/necrosis using puromycin (2 μg/ml). After several days, floater cells were collected and stained with PI to allow selective identification and recovery of cells that had lost membrane integrity. Using flow cytometry, PI⁺ (dead) cells were sorted directly into PCR tubes containing 25 μl of cell lysis buffer (50 mM KCl, 10 mM Tris-HCl, pH 8.0, 0.5% Tween-20, 0.5% Triton X-100, 2 mM MgCl₂, 1U/μl Proteinase K) and incubated at 1) 60° C. for 2 hours and 2) 95° C. for 10 minutes. Subsequently, 25 μl of the stock PCR reaction mix (50 mM KCl, 10 mM Tris-HCl pH 8.0, 400 uM-dNTP's, 2 mM MgCl₂) was added to each tube and PCR was carried out using primers (0.4 uM) specific for amplification of the retroviral GFP construct (OVT131, 5′ GACCTTCGGCGTCCAGTGCTTCAG; OVT179, (5′ AGCTAGCTTGCCAAACC TACA). As a control, live cells (negative for PI uptake) from an untreated culture were also sorted and used for PCR. Results show that genomic DNA present in PI positive cells was clearly able to act as a suitable template for PCR amplification (FIG. 10). Amplification of the GFP product from dead cells did not appear altered in size or quantity compared to the product amplified from live cells.

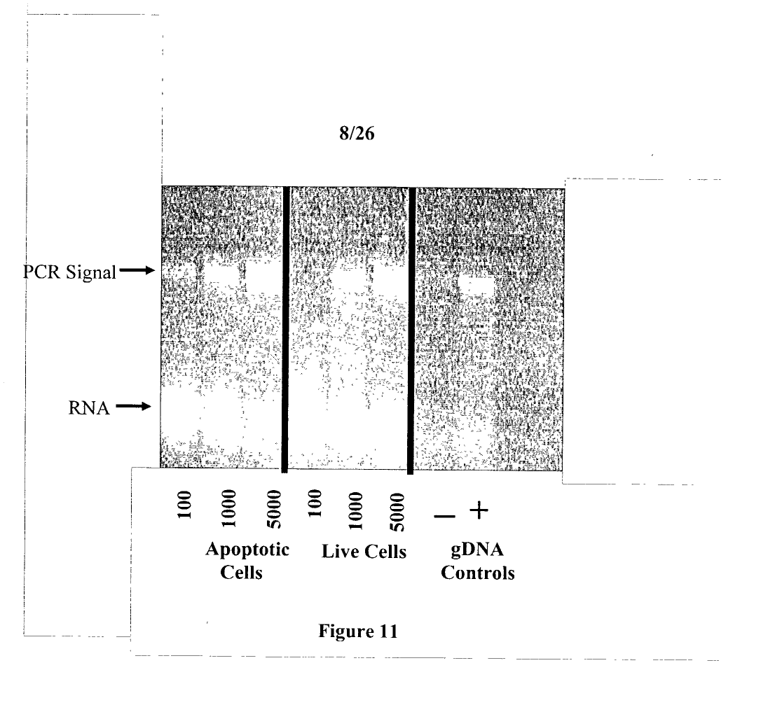

Cells that are positive for PI uptake may either be necrotic, or be in the late stages of apoptosis. In order to address specifically the question of whether DNA recovered from cells undergoing apoptosis can serve as a good template for PCR, the following experiment was performed. HT29 cells containing a retroviral construct that constitutively expresses GFP (pVT324) were treated with sulindac sulfide to induce apoptosis. After 48 hours of treatment, the majority of the cells had detached from the dish and showed typical apoptotic morphology (condensed nuclei). Apoptotic cells were counted into PCR tubes and PCR was carried out using primers specific for the amplification of the retroviral GFP construct. Live cells from an untreated culture were used as PCR controls. There was no apparent difference in amplification of the GFP product from apoptotic cells when compared to live cells (FIG. 11). Thus DNA recovered from cells undergoing either necrotic or apoptotic cell death can serve as an effective template for PCR amplification and construction of sublibraries.

### Example Four

### Negative Selection in HT29 Colon Cancer Cells

Twenty T175 flasks were seeded with 2.2×10⁶ HT29 cells/flask in McCoy's 5A media (Gibco BRL) modified with 10% FBS. On Day 1, each flask was infected (4 μg/ml polybrene, 50% volume) with a retroviral supernatant containing a commercially obtained brain cDNA library (“Example Three” above). On Day 2 the media was changed. On Day 3 both the floater and adherent cell populations were collected (separately) from the twenty flasks. Approximately 652,500 floaters were isolated from a theoretical background of 4.2×10⁷ adherents (1.5% floaters) and frozen down for future studies. Using the fluorescent properties of GFP as an indicator of infection, FACS analysis indicated that 76% of the viable cells were infected with the retroviral library. Additional floater cells were then collected Day 5, where the collection and counting procedures were repeated and some 7.8×10⁶ floaters and 7.6×10⁸ adherents were counted (1.03% floaters). The viable cell population was again scanned by FACS and the infection rate (GFP+) was found to 88%. The floater populations of Days 3 and 5 were then combined and readied for a genomic DNA prep using 16 a QIAamp kit (Qiagen) following standard procedures. Briefly, some 9×10⁶ floater cells in PBS were lysed to release gDNA. This material was passed over a QIAamp column that was then washed several times to remove protein and RNA contaminants. Twenty-seven micrograms of genomic DNA were then eluted from the columns with dH₂O and treated with RNAse A to eliminate any RNA contamination. This gDNA was then subjected to PCR procedures to amplify the library sequences encoded therein. Briefly, the above gDNA aliquot was divided into 27×1 μg samples, for use as templates for PCR using the oligonucleotides OVT 800 (5′ GCCGCCGGGA TCACTCTC) and OVT ¹²¹¹ (5′ GCTAGCTTGC CAAACCTACAGGTGGGG) (PCR conditions: 95° C., 30 seconds; 95° C., 15 seconds; 63° C., 30 seconds; 72° C., 3 minutes, cycle to “Step 2” twenty four times; 72° C., 5 minutes). The resulting PCR products were then divided into 5 pools, and each pool was then purified using QIAquick (Qiagen), digested with EcoRI and XhoI, and then directionally ligated into the original retroviral vector (pVT340). This material was then transformed into electrocompetent bacterial cells (DH10B, Gibco BRL) and plated out on LB-amp plates to create five distinct sublibraries. Each library was subsequently grown in liquid culture (LB+ampicillin) and processed (Qiagen Maxi Prep) to yield material for the second round of packaging in 293gp cells (see above). The resulting viral supernatants were then reinfected into naive HT29 cells (1×10⁶ cells per flask, three flasks per sublibrary) to begin the second round of negative selection. Round two and all subsequent rounds of the negative selection differ from Round 1 in that a) only single infections were performed and b) floater cells from Day 3 and Day 5 from each sublibrary were pooled together. Repeated cycling in this fashion yields library clones whose expression results in cell death.

TABLE 2
Percent Floaters

| | Mock | | | | | | |
|---|---|---|---|---|---|---|---|
| | infected | 324 | Pool 1 | Pool 2 | Pool 3 | Pool 4 | Pool 5 |
| Cycle 2 | 0.6 | 0.7 | 1.1 | 0.9 | 1.1 | 1.1 | 1.1 |
| Cycle 3 | 1.0 | 0.4 | 2.0 | 1.1 | 2.1 | 1.5 | 1.2 |
| Cycle 4 | 0.9 | — | 2.9 | 2.8 | 4.3 | 2.3 | 3.6 |
| Cycle 5 | 0.7 | 0.6 | 3.2 | 4.4 | 9.0 | 3.8 | 5.5 |
| Cycle 6 | 1.2 | 1.3 | 12.0 | 11.0 | 14.0 | 11.0 | 12.0 |

Results from six consecutive cycles of the negative selections are shown in Table 2, above, and are summarized as follows. Both mock infected cells and pVT324 control vector cells consistently show 1% (or less) floaters in the media. In contrast, all five pools show a 25 steady increase in the percent floater population over the course of the cycling with Pool 3 showing the greatest level of enrichment with 14% floaters in cycle six. This data demonstrates successful enrichment for perturbagen sequences that increase the frequency of dead and/or dying HT29 cells.

In addition to cycling these library sequences (obtained as described above) through an additional round of negative selections, 50 clones were taken from each of the Cycle 5, day five pools for sequence analysis. Two of these clones were found to encode N-terminal truncations of a known apoptotic protein. Clone #1 was reintroduced into fresh, naive HT29 cells and floater rates were compared with cells that had been mock infected or infected with the control vector, pVT324. Both controls exhibited low background floater rates of less than 1.5%. In contrast, HT29 cells infected with clone #1 exhibited roughly 18% floaters. In a similar experiment, clone #1 was introduced into HuVECs (Human Umbilical Vein Endothelial Cells, Clonetics/Biowhittaker) and cell viability was followed over the course of 16 hours. While control cells gave a background of 1% cell death at the 16 hour time point, 80% of the cells in the clone #1-infected culture died during the same period of time.

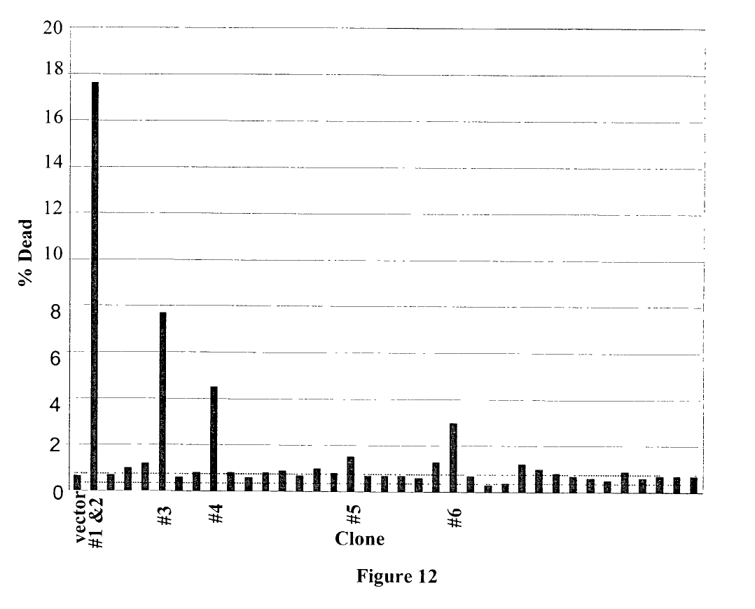

In addition to clones #1 and #2, four new cytotoxic agents have been identified from 36 clones picked at random (Sort VI). All four clones (Clones 3, 4, 5, and 6) give heightened levels of floaters in the HT29 floater assay (FIG. 12). Weaker cytotoxic agents (e.g. clones 5 and 6) give floater rates of 2-3% (respectively) while the more moderate cytotoxic agents (clones 3, and 4) induce between 4.5-7.5% floaters.

### Example Five

### Negative Selection in SW620 Colon Cancer Cells

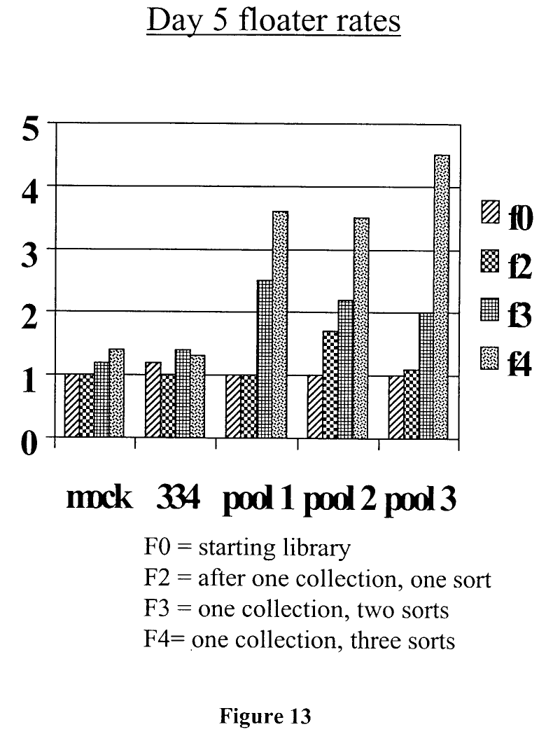

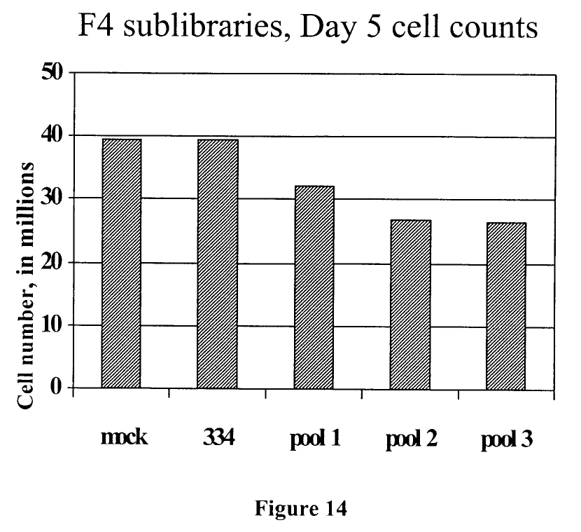

In a negative selection very similar to the HT29 screen described above, twenty flasks of SW620 colon cancer cells were plated (3 million cells/flask) and infected with one of two putative cytotoxic sequence-encoding libraries. The first library was made from random primed placental cDNA inserted into the MFG vector. The second library of putative cytotoxic agents was a random oligonucleotide library inserted into an internal site (insertion site 6, pVT27) of GFP (pVT 334, see Abedi et al. 1998). Following infection of these libraries into the SW620 cell line, floaters were collected at 48 and 96 hour time points (Days 3 and 5). These cells were then treated with propidium iodide (see above) and PI⁺ cells were sorted out by FACS. PI⁺ floater cells from both time points were then divided into three separate pools for a genomic DNA preparation. Subsequently PCR was used to amplify and recover the relevant perturbagen encoding sequences. Two unique sets of primers were used for PCR amplification; for the random primed placental library, OVT 1136 (5′ GGATCACTCTCGGCATGGACGAG) and OVT 1137 (5′ ATCCGCGGCC GCGGCCATAATGGCC) were used. For the random peptide (oligo) library, OVT 777 (5′ GACTGCCATGGTGAGCAAGGGC) and OVT144 (5′ GCCGTCCTCGATGTTG TGGCGGAT) were used. Results show that after performing four cycles of the infection and collection procedures (F4) in SW620 cells infected with the peptide library, the background level of Day 5 floater cells rose from approximately 1% in the original library to (on an average), 3.9% (FIG. 13). At the same time, background levels of floaters in the mock and pVT334 remained low at 1.35%. While the increase in background floater level was not accompanied with a concomitant increase in PI⁺ cells in the floater population, a decrease in the total cell number was observed over the course of the selection process, suggesting that one or more library sequence(s) that affect cell growth rates/cell viability are being enriched (FIG. 14). These potential cytotoxic agents (as well as those from earlier rounds of selection using the random primed placental library) are then reintroduced into naive SW620 cells and cycled again. Following 4-6 rounds of cycling, individual library inserts are sequenced and validated for cytotoxic activity.

### Example Six

### Negative Selection in T47D Metastatic Mammary Epithelial Cells

An additional example of floater assays involves the cell line T47D (ATCC) which is derived from a metastatic mammary epithelial cell tumor. T47D was chosen for study primarily due to the relatively low floater rate that it displays, and its ease of infection with retroviral based vectors.

To determine floater rates for the T47D cell line, cells were plated to 20% confluency in T175 tissue culture flasks (roughly 5×10 cells/flask) and the number of floaters as a percentage of total cells (adherent+floaters) was ascertained. Floater rates for T47D cells were determined to be 0.5% over a 3-5 day period in culture. In addition, 70% of the floater cells were observed to be dead as judged by trypan blue staining (“Handbook of Fluorescent Probes and Research Chemicals” Haugland, R. P., Molecular Probes). In contrast, less than one percent of the adherent cells were found to be dead using the same staining methods. Thus by harvesting floater cells from a T47D culture, at least 30% of the total number of dead and or dying cells are obtained. As the infection rate of this cell line with the pVT324 retroviral vector was observed to be approximately 90%, the T47D cell line thus was suitable for negative selections.

The T47D cell line is then utilized for a conditional negative selection—i.e., a selection in which cytotoxic agents that act under a unique set of conditions are identified. In this non-limiting Example, library sequences that enhance the sensitivity of T47D cells to the chemotherapeutic drug, camptothecin (an inhibitor of topoisomerase II), are selected as follows.

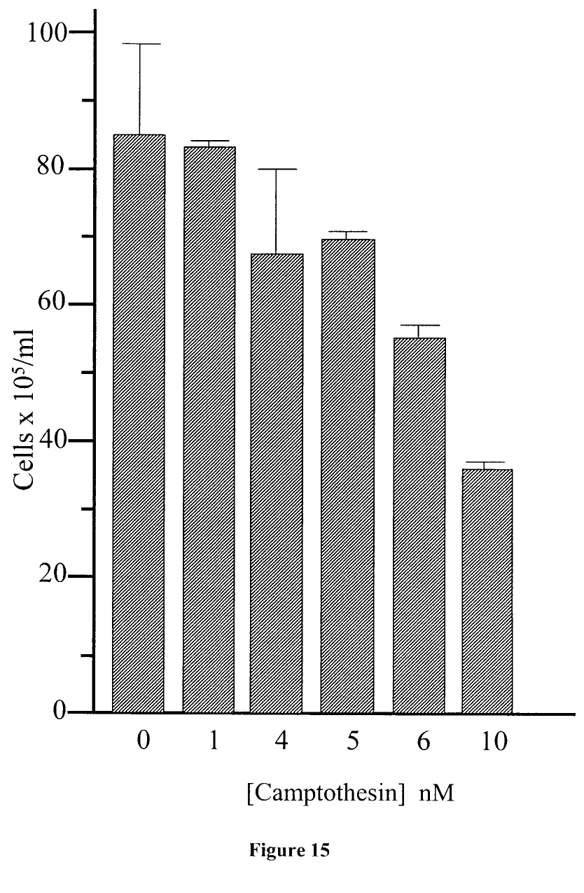

Initially, a maximal concentration of camptothecin that failed to increase T47D cell floater rate was determined as follows. Approximately 250,000 cells were seeded into each well of a six well plate. Cells were then grown in media containing camptothecin of varying concentrations (0-10 uM). After 5 days, the number of cells remaining in each of the camptothecin-treated wells was compared with untreated controls. From these experiments, it was determined that camptothecin concentrations ranging from 1-4 nM had no effect on T47D cell number over the course of the 5 days of treatment. Treatment of cells with concentrations greater than 4 nM resulted in a decrease in cell number relative to the untreated control (FIG. 15). As can be seen, cell number in the presence of 10 nM camptothecin was roughly one third that found in the untreated control, and virtually no cells remained adhered to the plate when exposed to camptothecin concentrations greater than 50 nM. These results suggest that T47D cells can tolerate camptothecin concentrations up to 4 nM without an adverse effect on either cell viability or division. In order to determine whether this level of treatment is concomitantly increasing the number of floaters in the population, several flasks are seeded with T47D cells and then treated with 1-4 nM concentrations of camptothecin. After a period of three to five days, the media is collected and the number of floater cells are counted and compared to the total number of cells in the flask (floaters+adherents).

To perform a conditional negative selection involving T47D cells, the following experiments are performed. Cells are infected with either a retroviral-based cDNA or peptide expression library as described for HT29 colon cancer assay. Following infection, cells are treated with 4 nM camptothecin and floater cells are harvested over a 5 day period. As was described previously, additional enrichment of library inserts encoding cytotoxic agents can be achieved by including in this protocol a PI staining/recovery (FACS) step that enables the identification of dead and/or dying cells. Library inserts present in these floater cells are recovered by PCR, subcloned into a retroviral vector, and subsequently reintroduced into naive T47D cells. Following this second infection, floater cells are again harvested over a five-day period in the presence of 4 nM camptothecin and the cycle is repeated. As was the case with the HT29 negative selection, repeated cycling in this manner should yield library clones whose expression results in cell death either in the presence or absence of camptothecin.

To identify the subset of library clones that cause cell death only in the presence of sub-toxic levels of camptothecin, one of two counterscreens is employed. First, the sub-library of inserts that cause cell death is introduced into T47D cells in the absence of camptothecin. Cells containing library clones that cause non-specific cell death will die, whereas clones that induce death only in the presence of camptothecin, will survive. To identify those clones that specifically increase the sensitivity of metastatic cells to camptothecin, a second counterselection is employed. Library inserts that cause camptothecin-specific death are introduced into primary mammary epithelial cell (Clonetics-Bio-Whittaker, Catalogue # α-2551), in the presence of sub-toxic levels of camptothecin. Library inserts present in cells that survive this treatment are then recovered by PCR, subcloned into the original host retroviral vector, and analyzed. Through the use of these two counter selections, cytotoxic agents that specifically increase the sensitivity of metastatic breast epithelial cells to the chemotherapeutic agent camptothecin are identified.

### Example Seven

### Negative Selections in HuVEC Cells

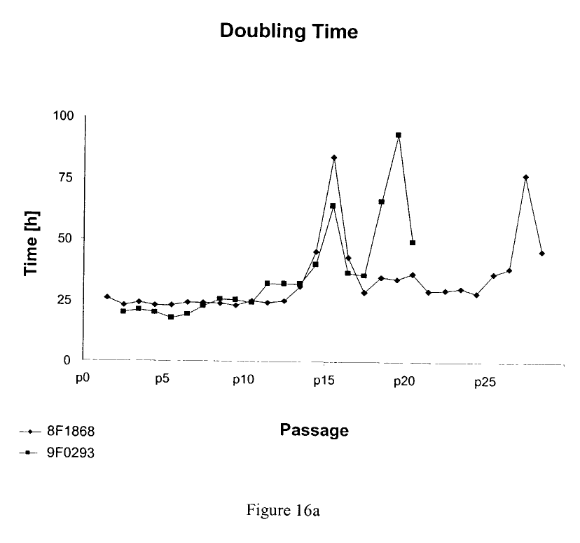

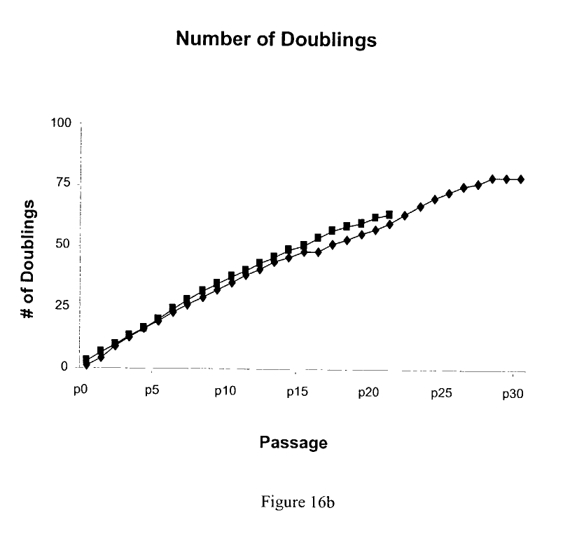

As an alternative to performing negative selections on transformed (immortalized) cells (e.g. HT29), protocols have been developed to apply the floater assay to primary cells. HuVECs (Human Umbilical Vein Endothelial Cells) are primary cells frequently used to pursue studies in angiogenesis. To prepare for negative selections in primary cells, two isolates of HuVECs, 8F1868 and 9F0293 (Clonetics/Biowhittaker) were plated in EGM-2 media (Clonetics/Biowhittaker) and observed over the course of several weeks to determine the doubling time and longevity of the cultures. Both lines exhibited a fairly consistent doubling period over the course of the first 10-12 passages (˜24 hrs). The life span of 9F0293 was limited to twenty passages with later passages (>12) exhibiting both broad fluctuations in doubling time and an alteration in morphology from cobblestoned, epithelial-like cells to a more flattened, fibroblast-like morphology. In contrast, the 8F1868 line had a life span that extended to 30 passages and showed a greater consistency in doubling time. Because these two cultures performed identically during the first six passages and because the proposed negative selections would take place during passage four, line 9F0293 was chosen for future negative (FIG. 16a,b).

To assess the feasibility of using primary cell lines in negative selections, the 9F0293 line was tested for a) susceptibility to retroviral infection and b) the background percentage of floater cells. Three samples of an early passage of 9F0293 cells (control, mock infected, and infected) were plated at a density of 2×10⁵/15 cm² plate and followed over the course of 120 hours. During that time the total cell number, doubling time, and floater ratios (calculated here as total # of floaters/total # of adherents) were recorded and compared. Cells were infected with pLIBEGFP (Clontech) and packaged using the CaCl₂ protocol described previously. Retroviral infection protocols used in these procedures included a 12 hour period of infection using an MOI (moiety of infection) of 2.0, and 4 ug/ml of polybrene.

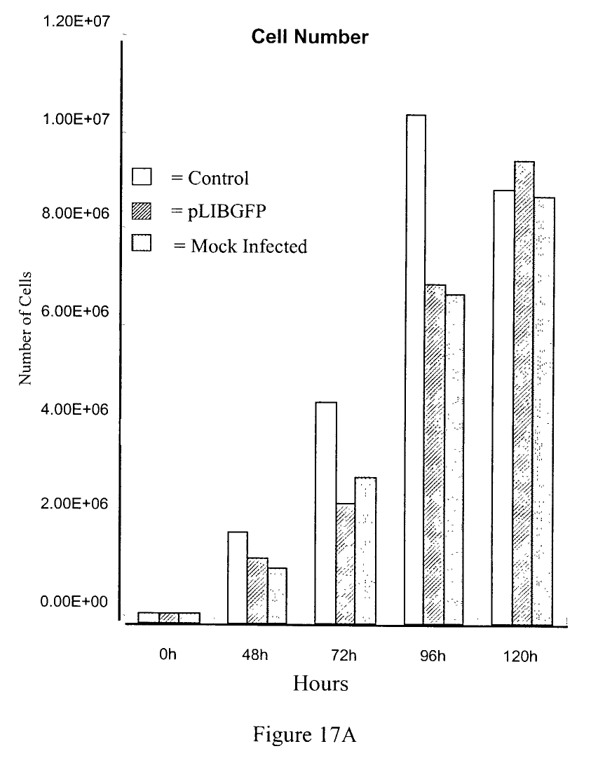

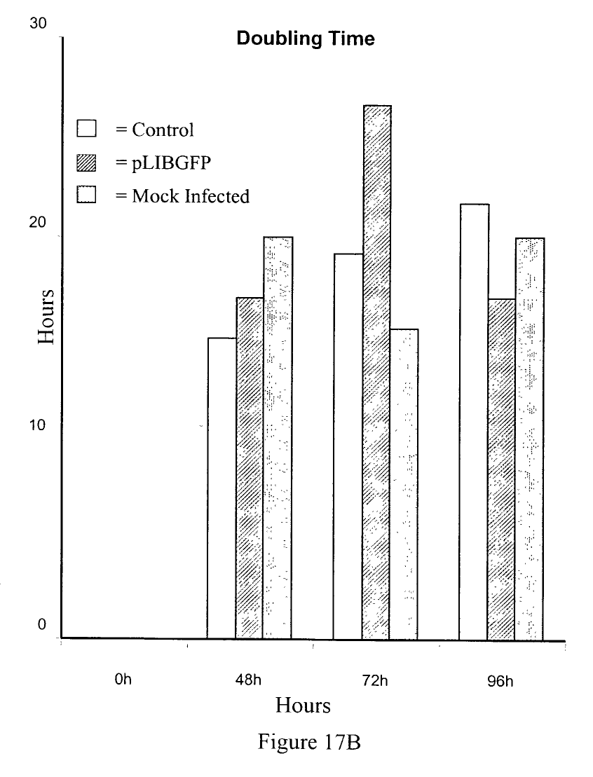

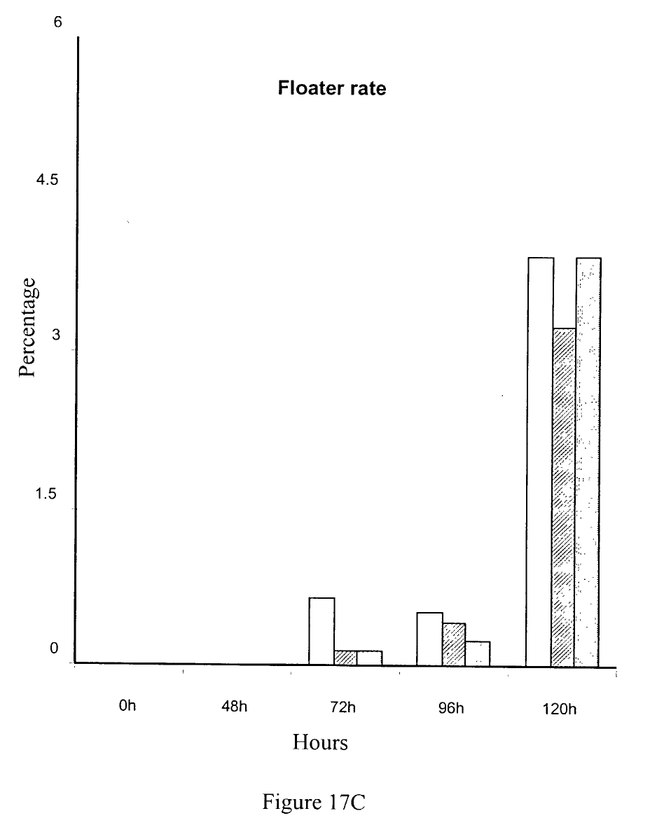

Results show that when compared to the controls, the infection procedure and presence of retrovirus altered the total cell number and doubling time of the 9F0293 line only slightly (FIG. 17a,b, c). In addition, at all points prior to 120 hours, floater ratios in all three scenarios were consistently below 1%. At 120 hours, cell cultures were confluent and floater ratios increased (3% or greater), an observation that is consistent with nearly all mammalian cell cultures studied thus far. In addition, the 9F0293 line of HuVECs proved to be highly susceptible to retroviral infection, with the percentage of cells falling into the GFP⁺ gate averaging between 70-80%.

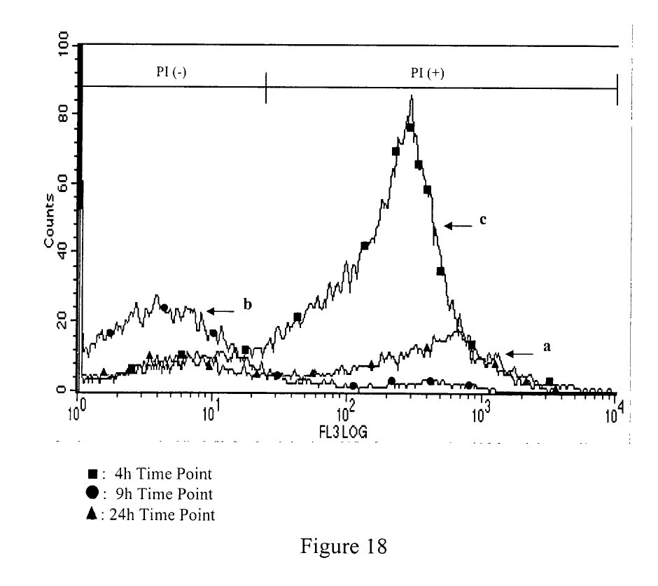

To determine the time course in which dead and/or dying HuVECs detach from the solid support in response to a cytotoxic agent, 9F0293 cells were plated and subsequently treated with puromycin (2 ug/ml). Both floater and adherent cell populations were then collected at varying times (t=4, 7, 9, 16, 20, and 24 hours) and analyzed to determine (a) the percent PI⁺ cells in the floater population and (b) the fraction of cells which became floaters. The results of these experiments showed that the majority of HuVECs detached by the 16-hour time point and that 90-99% of the cells became floaters within 24 hours after addition of puromycin. The fraction of floaters that stained with PI (PI⁺ population) increased with time. At early time points (4, 7, and 9 hours), PI⁻ and PI⁺ floater cells were in near equal numbers. By 20 hours, nearly all the floater cells fell into the PI⁺ gate, suggesting HuVECs detach as PI⁻ cells and then rapidly convert to the PI⁺ phenotype (FIG. 18).

To identify cytotoxic agents that induce apoptosis/necrosis in HuVECs, 9F0293 cells are plated on a solid support (gelatine) and infected (MOI=2, 16 hr infection, 4 ug/ml polybrene) a retroviral-based library of inserts which use the backbone of pCLMFG (see “Example 3, Section B” above) or PLIB (Clontech) as the retroviral vector. Either random oligonucleotides inserted into the VT27 loop of GFP (see above, Abedi et. al. 1998), cDNA, or genomic DNA fused to the C-terminus of GFP (see “Example 3, Section B” above), are screened for cytotoxic agents.” At 48 hr, 72 hr, and 96 hr time points post infection, floater cells are collected. Floaters from the two earliest collection points are pooled together (Pool 1). Floaters from the 96 hr time point form a separate pool (Pool 2). Genomic DNA prepared from each pool is then used to amplify the library inserts using standard PCR techniques (see above). The product from this reaction is then recloned into the appropriate retroviral vector, and reinfected into naive 9F0293 cells for subsequent rounds of screening and enrichment. In later rounds of screening (>4), individual perturbagen clones are isolated, reintroduced into HuVECs, and tested to determine if such library inserts increase the level of floater cells above the background floater rate observed in uninfected and mock infected cultures.

Library inserts found to be cytotoxic in HuVECs are then introduced into additional cell types to determine the cell or tissue-type specificity of the encoded agents. Specifically, the encoding cytotoxic agents are introduced into HT29, SW620, DLD-1, as well as other cell lines (both primary and genetically altered so as to be immortalized or transformed). The levels of cell death are monitored using any one of (but not limited to) the techniques described in “Example One”.

### Example Eight

### Negative Selection in Transformed Primary Human Mammary Epithelial Cells (HMEC)

A floater assay involving primary human mammary cells (HMECs), immortalized and transformed with a minimum number of genetic elements, was performed as follows. Primary pooled HMECs (Biowhittaker/Clonetics) were immortalized and transformed with two methods: 1) Serial introduction and expression of retroviral constructs encoding SV-40 Lg T Ag, human telomerase catalytic subunit hTERT, and activated V12H-ras (method of Hahn, et al, Nature 400: 464-468, 1999) and 2) serial introduction and expression of constructs encoding HPV-16 E6/E7, hTERT and V12H-ras. The lines are designated 96C and 96A, respectively. The retroviral constructs BABE/neomycin/SV-40 Lg T Ag BABE/LNCX/puromycin/hTERT, LXSN/hygromycin/V-12H-Ras and LXSN/neomycin/HPV-16 E6/E7 were utilized.

To determine background floater rates, cells were plated to 20% confluency in three 100 mm dishes (100000 cells/dish) and the number of floaters as a percentage of total cells (adherent+floaters) and the viability of floaters ascertained at two, three and four days post-platin . Viability was assessed by measuring propidium iodide uptake. In addition to untreated controls, a transduction control vector pVT 340 (pCLMFG/GFP) and a positive control BID/MFG vector, a pro-apoptotic protein previously identified in a colorectal carcinoma HT29 negative selection, were utilized. Cells were transduced with 30% (v/v) viral supernatants at 95% and 45% rates, respectively.

TABLE 3
Percent Floaters

| | HMEC 96A | | | HMEC 96C | | |
|---|---|---|---|---|---|---|
| | Mock | | | Mock | | |
| | infected | 340 | BID | infected | 340 | BID |
| DAY 2 | 0.7 | 0.9 | 54 | 0.6 | 0.2 | 46 |
| DAY 3 | 0.9 | 0.9 | 25 | 0.7 | 1 | 58 |
| DAY 4 | 0.4 | 0.7 | 8 | 0.4 | 0.3 | 7 |

TABLE 4
HMEC 96A

| | Propidium Iodide Staining | | | | | |
|---|---|---|---|---|---|---|
| | Mock | | 340 | | BID | |
| | Adherent | Floaters | Adherent | Floaters | Adherent | Floaters |
| DAY 3 | 1.6 | 36 | 1.2 | 44 | 1.6 | 72 |
| DAY 4 | 0.3 | 36 | 0.5 | 28 | 0.4 | 45 |

TABLE 5
HMEC 96C

| | Propidium Iodide Staining | | | | | |
|---|---|---|---|---|---|---|
| | Mock | | 340 | | BID | |
| | Adherent | Floaters | Adherent | Floaters | Adherent | Floaters |
| DAY 3 | 0.2 | 45 | 0.5 | 36 | 8.5 | 81 |
| DAY 4 | 0.8 | 35 | 0.2 | 36 | 0.5 | 70 |

Floater rates for both 96A and 96C were determined to be 0.4-0.9% for naive cells and 0.7-0.9% for 340 vector control transduced cells, whereas BID infected positive controls gave a floater rate for 54% on day 2 for line 96A and 46% on day 2 for line 96C (See Table 3). In addition, 28-44% of floaters compared to 0.5-1.2% for adherent line 96A cells were judged to be non-viable with propidium iodide staining on days 3 and 4. In line 96C, 36% of floaters and 0.2-0.5% of adherent cells stained with propidium iodide on days 3 and 4. BID transduced cells were 45-75% propidium iodide positive in floaters and 0.4-1.6% positive in adherents in line 96A and 70-81% in floaters and 0.5-8.5% propidium iodide positive in line 96C (see Table 4 & 5). A high viability adherent cell component and low viability floater cell component coupled with low floater rates reflects positively on the suitability of these lines for negative selections. The floater assay also performed well with the positive control BID, with a majority of nonviable cells detaching. Thus, by collecting floaters in this assay a majority of cell-death inducing perturbagen sequences should be recovered.

### Example Nine

### Identifying Agents That Overcome Multidrug Resistance

The present invention may be readily applied to identify agents that sensitize multidrug resistant (MDR) cancer lines to currently available chemotherapeutic agents. One nonlimiting example is as follows.

Many MDR strains (e.g. LS513, LS1034) can be obtained through ATCC. Alternatively, MDR strains can be obtained by the following, non-limiting procedure. Ten T75 flasks containing 2×10⁶ HT29 cells/flask are subjected to a drug (e.g. taxol) at concentrations that induce 90-95% cell death. Following this treatment, the surviving cells are allowed to expand in normal media, whereupon they are subjected to elevated levels (e.g. 5×) of the drug. As a result of multiple cycles of killing, regrowth, and stepwise increases in drug concentrations, an HT29 MDR strain is evolved.

Prior to performing a screen for perturbagens that sensitize multidrug resistant (MDR) cancer lines to currently available chemotherapeutic agents, it is critical to first determine a sublethal concentration of the drug to be used in the studies. In this example, a “sublethal” dose is a concentration of a drug that is capable of killing an MDR⁻ cell line, but has little or no effect on an MDR⁺ line. In one non-limiting example of how a sublethal concentration can determined, killing curves are performed on both LS513 (an MDR⁺ line) and several MDR⁻ control lines. Specifically, 1×10⁶ LS513 cells are plated in 15 cm² plates and allowed to adhere overnight. On the following day, taxol is added to the culture at a range of concentrations varying from 2 nM-500 uM. The cells are then cultured for an additional 2-7 days, whereupon the cell number and floater rates are compared. A sublethal concentration of taxol is then defined as a concentration of the drug that that kills greater than 50% of the MDR⁻ (control) cells but induces less than 2% lethality in the LS513 line.

To identify sequences that disrupt the MDR phenotype, library inserts (either cDNA or random peptide based) are introduced into adherent MDR lines (e.g. LS513 and LS 1034 colorectal carcinoma cell lines, ATCC) using the retroviral technology described above. These cells are then subjected to sublethal concentrations of chemotherapeutic drugs (e.g. taxol, adriamycin, vinblastine, actinomycin) and cultured over a period of two to seven days. As the majority of cells do not contain a library insert that will enable the drug to overcome or disrupt the mechanism of multidrug resistance (e.g. P-glycoprotein), these cells will continue to divide and remain adherent to the solid support. To identify library inserts that enhance the sensitivity of MDR cells to chemotherapeutics (essentially converting an MDR⁺ line to MDR⁻) floater cells are collected over the course of the experiment. Again, as described in previous sections, additional enrichment of cytotoxic perturbagens with these characteristics can be achieved by including in this protocol a PI staining/recovery (FACS) procedure that enables the identification and recovery of dead and/or dying cells. The sequence(s) are then recovered and amplified from floater cell genomic DNA preparations via PCR and recycled through an additional round(s) of selection to enrich for perturbagen sequences that disrupt the MDR phenotype.

To identify the subset of library clones that cause cell death only in the presence of sub-toxic levels of taxol, a counter screen is employed. The sub-library of inserts that cause cell death is introduced into LS513 cells in the absence of taxol. Cells containing library clones that cause non-specific cell death will die, whereas clones that induce death only in the presence of taxol will survive.

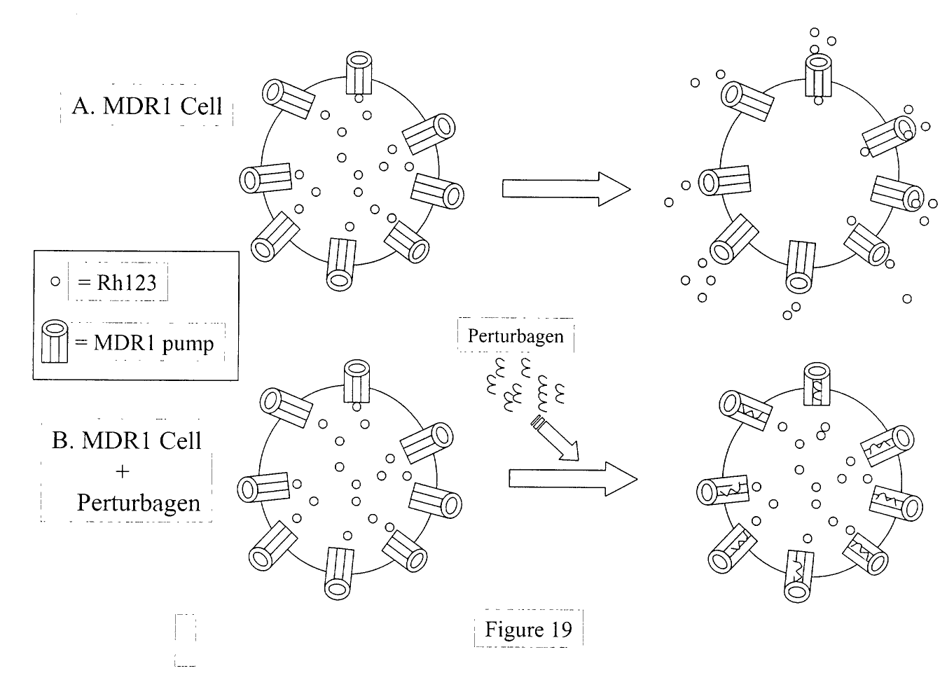

In many MDR cell lines, the drug resistant phenotype has been associated with over-expression of P-glycoprotein (MDR1), a cytoplasmic membrane associated protein that is capable of removing (or pumping) a wide variety of chemotherapeutic drugs from the cell cytoplasm to the extracellular space To enrich for agents that disrupt the pumping action of P-glycoprotein, MDR1 strains are infected with libraries encoding putative and grown in sublethal concentrations of taxol. These cultures are then exposed to Rhodamine 123 (Rh123), a membrane-permeable, fluorescent substrate of P-glycoprotein. Subsequently, floater cells are collected and sorted on the basis of fluorescence. Cells that exhibit a “dim” phenotype by FACS are capable of removing Rh123 from the cytoplasm and thus have an active P-glycoprotein pump (FIG. 19). These cells do not contain an agent that interferes with the P-glycoprotein pump action and are discarded. Cells that are “bright” accumulate Rho123 in the cytoplasmic compartment, and thus contain an agent that disrupts the function of the MDR1 pump. In this Example, the term “disrupts” can refer either to molecules that directly interfere with the action or activation of the MDR1 pump, or to molecules that alter or prevent the localization of P-glycoprotein to its native site. These Rh123 “dim” cells are collected by FACS and recycled through additional rounds of selection (see above) to enrich for sequences that interfere with the pumping action of P-glycoprotein.

As an alternative to this assay, detection of agents that disrupt the action of P-glycoprotein can be performed in the absence of the chemotherapeutic drug. Under these circumstances, cultures of MDR1 cells are infected with a library of inserts encoding putative disruptive agents, cultured for a brief period (24-72 hrs) to allow expression of the library inserts, and treated with trypsin to release the cells from the solid support. The cells are then exposed to Rh123. and sorted by FACS to identify Rh123+cells (cells that are unable to pump Rh123 out of the cell) within the population. The library insert(s) encoding these agents are then PCR amplified and recycled through additional rounds of selection to enrich for sequences that interfere with the product of the MDR1 gene.

### Example 10

### Somata and Negative Selections Preparation of a cDNA Library

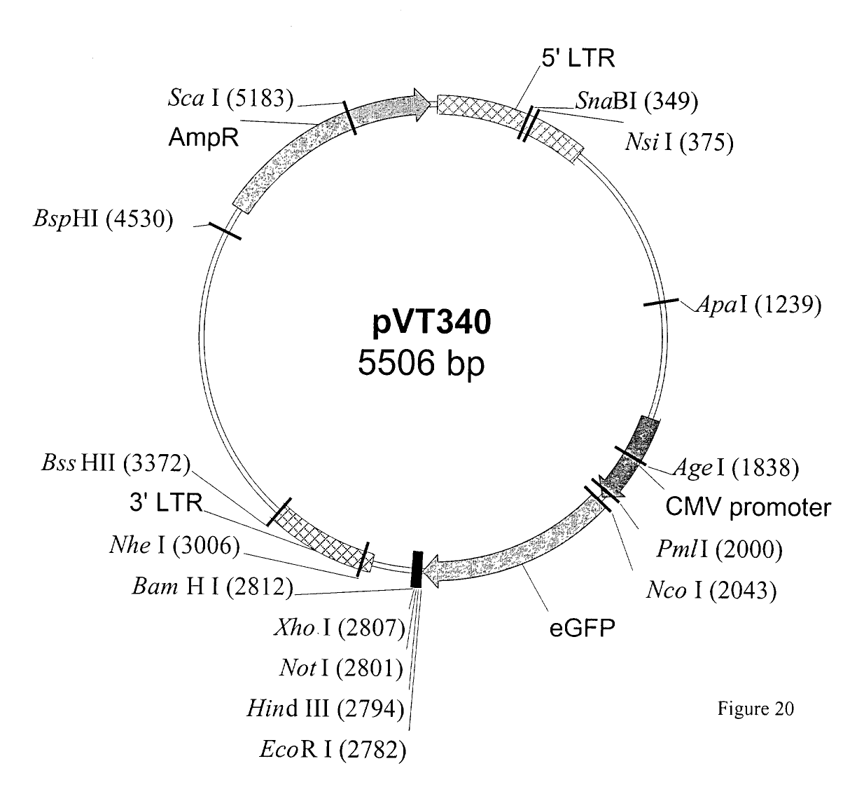

To construct a retroviral vector that was appropriate for negative selections, the 3.8 kB HindIII/ScaI band of pVT314 was ligated to the 1.9 kB SSPI/PvuII band of pBluescript™ (Stratagene). The final product of this reaction (referred to as pVT340, see FIG. 20) contains all the necessary components of a constitutive retroviral expression vector including a Psi site for packaging, constitutive CMV driven perturbagen expression, a splice donor and acceptor site for obtaining high levels of library insert expression, and a multiple cloning site (MSC) linked to the 3′ end of EGFP. As a source of perturbagens, a cDNA library obtained from human brain tissue was purchased from Origene Inc. (Catalogue # DHL101, DHL 105, and DHL 106) and transformed into bacteria (DH10B, Gibco). Using standard techniques, bacterial hosts carrying the libraries were then expanded in liquid media (LB plus ampicillin) and used to prepare large quantities of episomal (library) DNA (Maxiprep, Qiagen). The cDNA insert in each vector was then released by digestion with the appropriate restriction enzymes (EcoRI/XhoI) and the fragments measuring 0.4-2.8 kB were gel purified and ligated (T4 Ligase, Boehringer Mannheim) into the compatible sites of the pVT340 retroviral vector. As a result of these procedures, putative cytotoxic agents are expressed constitutively as fusions with the GFP scaffold.

Retroviral Packaging and Infection

Library constructs were packaged for retroviral transfection into the cell of choice using LipofectAMINE. Specifically: On Day 1, 3×10⁶ cells of the packaging cell line (293gp) are seeded into a T175 flask. On the second day, two tubes, one carrying 15 ug of library DNA +10 ug of envelope plasmid (pCMV-VSV.G-bpa)+1.5 ml DMEM (serum free), the second carrying 100 ul of LipofectAMINE (Gibco BRL)+1.5 ml DMEM (serum free) are mixed and left at room temperature for 30 minutes. Subsequently, the two tubes are mixed together along with 17 ml of serum free DMEM. This cocktail is referred to as the “transfection mix.” Previously plated 293gp cells are then gently washed with serum free media and exposed to 20 ml of the transfection mix for 4 hours at 37° C. Following this period, the transfection mix can be removed and the cells are incubated with complete DMEM (10% serum) for a period of 72 hours at 37° C. On Day 4 or 5, the media (now referred to as “viral supernatant”) overlying the 293gp cells is collected, filtered through a 0.45 μ filter and frozen down in at −80° C.

As an alternative to the LipofectAMINE method of retroviral DNA packaging, a second protocol, referred to herein as the “CaCl₂ Method,” can be used to package retroviral sequences. In this method, 5×10⁶ cells of the packaging cell line (293gp) are seeded into a 15 cm² flask on Day 1. On the following day, the media is replaced with 22.5 mls of modified DMEM. Subsequently, a single tube carrying 22.5 μg of retroviral library DNA and 22.5 μg of envelope expression plasmid (pCMV-VSV.G-bpa) is brought to 400 μl with dH₂O, to which is added 100 μl of CaCl₂ (2.5M) and 500%1 of BBS (dropwise addition, 2× solution 50 mM, BES (N,N-bis(2-hydroxyethyl)-2-aminoethane-sulfonic acid), 280 mM NaCl, 1.5 mM Na₂HPO₄, pH 6.95). After allowing this retroviral mixture to sit at room temperature for 5-10 minutes, i.e. is added to the 293gp cells in a dropwise fashion, and the cells are then incubated at 37° C. (3% CO₂) for 16-24 hours. The media is then replaced and the cells are allowed to incubate for an additional 48-72 hours at 37° C. At that time, the media containing the viral particles is then collected, filtered through a 0.45 μ filter and frozen down at −80° C.

Floater Cell Assays in HT29 Colon Cancer Cells

In the first round of selection, twenty T175 flasks were seeded with 2.2×10⁶ HT29 cells/flask in McCoy's 5A media (Gibco BRL) modified with 10% FBS. On Day 1, each flask was infected with a retroviral supernatant (4 μg/ml polybrene, 50% volume) containing the cDNA library and on Day 2 the media was changed. Using the fluorescent properties of GFP as an indicator of infection levels, FACS analysis was performed on a sample of the population. In cycles 1-6, approximately 90% of the viable cells were observed to be infected with the retroviral library. In cycle 7, cells were intentionally infected with low moieties of infection (MOI) to ensure single inserts in each cell. As a result, FACS analysis showed that only 5% of the cells contained a viral insert.

On Days 3 and 5, floater cells (totaling roughly 1% of the total population) from the original twenty flasks were divided into three separate subpools (I, II, and III), and readied for a genomic DNA prep using a QIAamp kit (Qiagen). Briefly, floater cells were centrifuged into a pellet (400× g), washed in PBS, and lysed to release gDNA. This material was then gravity filtered over a QIAamp column designed to bind/retain genomic DNA. The column was then washed several times to remove protein and RNA contamination, and the genomic DNA was then eluted with dH₂O and treated. Genomic DNA samples were then ethanol precipitated, washed, and treated with RNAse A to eliminate any RNA contamination.

Floater cell gDNA was then subjected to PCR procedures to amplify the library sequences encoded therein. Briefly, the above gDNA aliquot was divided intol 1 μg samples for use as templates for PCR using the oligonucleotides OVT 800 (5′GCCGCCGGGATCACTCTC) and OVT 1211 (5′ GCTAGCTTGC CAAACCTACAGGTGGGG) (PCR conditions: 95° C., 30 seconds; 95° C., 15 seconds; 63° C., 30 seconds; 72° C., 3 minutes, cycle to “Step 2” twenty four times; 72° C., 5 minutes). The resulting PCR products were then purified using QIAquick (Qiagen), digested with EcoRI and XhoI, and then directionally ligated into the original retroviral vector (pVT340). This material was then transformed into electrocompetent bacterial cells (DH10B, Gibco BRL) and plated out on LB-amp plates. Each sublibrary was subsequently grown in liquid culture (LB+ampicillin), processed to yield plasmid DNA (Qiagen Maxi Prep), and repackaged in 293gp cells. The resulting viral supernatants were then reinfected into naïve HT29 cells (1×10⁶ HT29 cells per flask, three flasks per subpool, 50% supernatants) to begin the second round of negative selection. Cycles II-VI used protocols and techniques similar to those described above. In Cycle VII, both the numbers of cells plated and percent viral supernatant used in the infection the cells were reduced (Cycle VII: 2 T75 flasks per subpool, 6.5×10⁵ cells per flask, Flask #1=10% supernatant, Flask #2=1% supernatant).

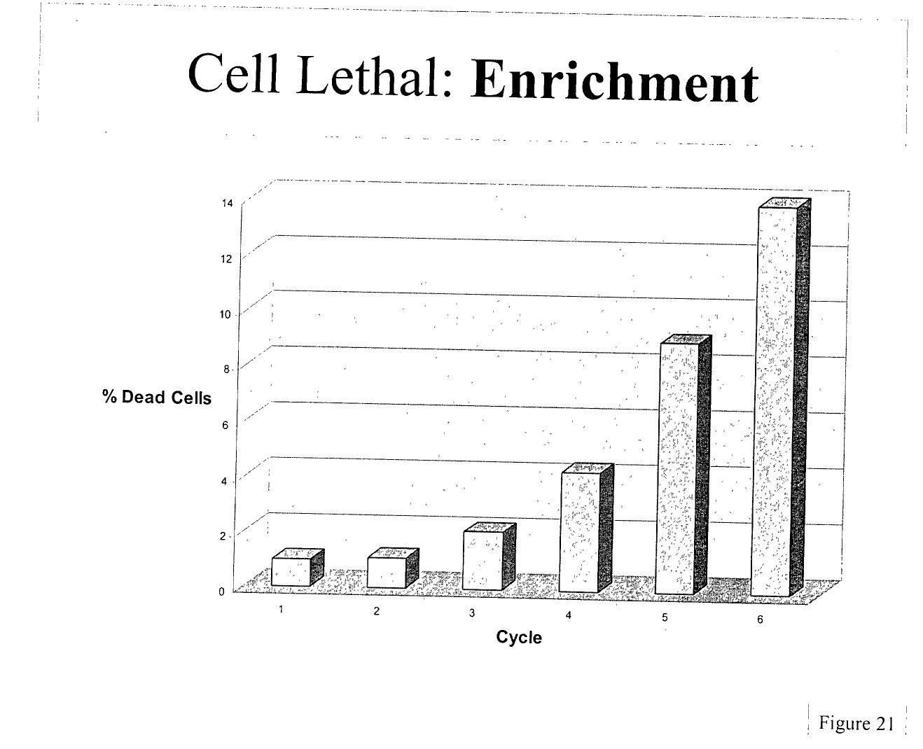

Results from seven consecutive cycles of floater selections are summarized as follows: i) both mock infected cells and pVT340 control vector cells consistently show 1% (or less) floaters in the media and ii) all three subpools exhibited a steady increase in the percent floater population over the course of the cycling (FIG. 21). On average, the number of floaters grew to roughly 14% by the end of Cycle VII. This data demonstrates successful enrichment for perturbagen sequences that increase the frequency of dead and/or dying HT29 cells.

Thirteen hundred and forty seven clones derived from Cycle VII were sequenced and analyzed. The sequence sets revealed high enrichment for two fragments of a known apototic protein. By cycle 7, over 20% of the individual clones in the sub-library encoded one of the two in-frame fragments of the pro-apoptotic protein. When re-tested, these clones produced a marked increase in floater percent compared with controls. Moreover, when the sequence of clones taken from multiple stages of the enrichment procedure were compared, it was found that one of the two pro-apoptotic clones increased nearly 36-fold in abundance between cycle 6 and 7, thus demonstrating the validity of cycling as an enrichment procedure.

The predominance of these two clones in the later stages of the enrichment procedures suggested the possibility that weaker clones may have been lost during the final selection cycles, perhaps excluded by the strong pro-apoptotic clones. This disparity in representation should be exponential (a power of cycle number), and should be less pronounced in earlier cycles. Thus, the cycle 4 sublibrary, the first one in which significant increases in floater rate were observed, was chosen as a source of cytotoxic/cytostatic perturbagens.

Because of the low floater rate displayed by cells transduced with cycle 4 material, and the likely corresponding low frequency of cell-lethal clones, a thorough search for clones, both weakly penetrant ones and strong ones, necessitated development of a large-scale system with the capacity to analyze thousands of single sequences. To solve this throughput problem, an automated genetic analysis system for mammalian cells called Somata was developed (See USSN#60/305,712 “Automated Assay Methodology”) the contents of which are incorporated herein. Briefly, Somata automation was configured with the following tools: i) a bacterial colony picker (AutoGenesys, Autogen, Framingham, Mass.) ii) a microtiter plate hotel/incubator (Cytomat 6000, Kendro Laboratory Products, Newtown, CT), iii) a 96 channel pipettor (Multimek™ 96, Beckman Instruments, Inc., Fullerton, Calif.), iv) a microplate hotel (Platestak™, CCS Packard, Torrance, Calif.), and v) a plate handler (Pick and Place, Fiore Automation, Salt Lake City, Utah). Custom ActiveX™ servers from Fiore Automation controlled each robotic instrument and were integrated by an instrument management software package, Capitano (Fiore Automation, Salt Lake City, Utah) that allows the end user to create, save, and execute sophisticated process control protocols.

To prepare retroviral DNA from bacterial minipreps, 5 ul of each culture, derived from, frozen glycerol stocks was inoculated into 0.8 mls of Terrific Broth and incubated/shaken at 37° C. in a sterile, 96 well block capable of holding a total volume of 2 mls. After culturing the samples overnight, the blocks are centrifuged for seven minutes (3000 rpm) and the supernatant is decanted, leaving the bacterial pellet. Each sample is then resuspended in 90 μl of Qiagen Resuspension buffer and vortexed until the pellet is resuspended in solution. Subsequently, a lysis buffer (Qiagen Lysis Buffer, 90 μl/well) is added to each well, and the samples are gently mixed to lyse the bacterial cell wall. The samples are then incubated at room temperature for 2-4 minutes before adding 90 ul of Qiagen's neutralization buffer. Following centrifugation (4000 rpm, 7 minutes) the plasmid preparations contained in the lysate are then cleaned using the Millipore Montage system. Briefly, a Plasmid Plate (PP) is placed on a vacuum manifold and fitted with an adapter. Subsequently, a lysate clearing plate (LCP Mananly50, Millipore) tailored to fit the adapter, is placed on top. The lysate is then carefully removed from the deep well block containing the cell wall/protein pellet, and transferred to the LCP. A vacuum is then applied to the system (5 minutes) and the cleaned sample is captured in the Plasmid Plate below. Each well of the LCP is then washed (2×100 μl ddH₂O) followed by the addition of fifty microliters of Qiagen Elution Buffer to each well of the PP. The Plasmid Plate is then placed on a shaker for five minutes to recover the plasmid.

Highly infective retroviral supernatants were prepared by introducing each plasmid (along with the appropriate co-vectors, e.g.VSV-G envelope expression plasmid) into HS293gp packaging cells plated in either the 96-or 384 well format. As one non-limiting method of accomplishing this, 2×10⁵ early passage 293gp cells (gift of I. Verma, Salk Institute) suspended in 180 ul of media (DMEM⁺ media=DMEM+110% FCS+L-glut, 2 mM final) were plated into each well of a 96 well microtiter dish using either automated (Sorval “Cytomat” and a Beckman “Multimek” instrumentation) or non-automated techniques. The cells were incubated overnight to allow attachment to the solid support and then transfected with the retroviral miniprep DNA. Several viable methods were used to transfect cells (e.g CaCl₂, Lipofectamine, Transit™). In the CaCl₂ method, 133 ng of library plasmid DNA was mixed with 534 ng of envelope plasmid in a total volume of 5□1. CaCl₂ was added (5□1) to a final concentration of 250 mM, followed by an equal volume (10□1) of 2× BBS (50 mM BES(N,N-bis(2-hydroxyethyl)-2-aminothane-sulfonic acid), 280 mM NaCl, 1.5 mM Na₂HPO₄, pH 6.95). The solution was mixed every 5 minutes for 20 minutes before adding 20□1 (dropwise) to the wells containing the gp293 cells. The cells were allowed to incubate 16 hours at 37° C., and the media was replaced with 100□1 of fresh media. At 72 hours post transfection, the supernatant was removed to an empty 96 well plate, exposed to multiple freeze/thaw cycles (or filtration) to remove potential contaminant 293gp cells, and then frozen at −80° C. until used for transduction. Alternatively, 31.5 ul of 2× BBS (50 mM, BES (N,N-bis(2-hydroxyethyl)-2-aminoethane-sulfonic acid), 280 mM NaCl, 1.5 mM Na₂HPO₄, pH 6.95) could be added to a microtiter well containing 31.5 ul of 0.5 M CaCl₂, sample plasmid, and envelope expression plasmid, (pCMV-VSV.G) (Chen et al). One third of this mixture, 21.5 ul, is then added to the 293gp cells and treated in a similar fashion as previously described.

To begin the bioassay, retroviral supernatants encoding each cDNA were transduced into the host cells. Transductions into the host cells were performed by plating cells in a microtiter plate in a total volume of ˜100 ul media and allowing cells to attach over the course of several hours. Subsequently, the retroviral supernatants were thawed, filtered through a 0.45 um Multiscreen-HV, sterile filter plate (Millipore Corp., Bedford, Mass.) and added to the cells along with polybrene (4 ug/ml). In most instances, the viral supernatant represented 50% of the volume of the final mixture. The media was changed 14 hours after transduction and cells were cultured for a varying number of days (2-5 days) before performing the subsequent bioassay. In general, to test each cDNA for cytotoxic/cytostatic properties, the cells containing each agent were stained with a dye capable of detecting cells that have a compromised membrane (i.e. dead/dying cells). As one non-limiting example, the following procedures are followed: five days after introducing the cDNA into the cell culture, Sytox Orange is added to each well of the assay plate to a final concentration of 1 uM. The plates are then allowed to incubate for 30 minutes at 37° C., and then analyzed on a CCD imaging system (e.g. Sytox Orange, Ex:535+/−15 nm, Em 585+/−20 nm) to determine the total number of cells having a compromised cell membrane (i.e. dead cells). In this instance, the imaging system is composed of a PixelVision Spectra Video™ Series imaging camera (1100×330 back-illuminated array, Pixel Vision, Tigurd, Oreg.), Pixel Vision PixelView™ 3.03 software, two 50 mm/f2 Olympus macro focusing lens mounted front to front, four 20750 Fostec xenon light sources (Schott-Fostec, Auburn, N.Y.), four 8589 Fostec light lines, a 4457 Daedal stage, and supporting mechanical fixtures. Following these procedures, saponin is added (0.1% saponin, 30 minutes) to each well to permeabilize the remaining cells and an additional readout is performed to determine the total cell number. The number of dead and live cells in each well is then compared with the appropriate controls to assess if any cytotoxic/cytostatic properties are associated with the agent under study. Furthermore, the overall cyto-inhibitory nature of each agent was described by it's relative “kill index” which is the ratio between the number of dead cells and the total cell number.

In addition to developing the above-described Somata assay, new vectors were constructed to test the presumptive cytotoxic/cytostatic agents. To maximize expression level and hence sensitivity of the assay, the HT29 cell line was modified with an expression cassette that encoded the HIV Tat gene product. To accomplish this, the pVT313 construct was digested with BamHI and ClaI to remove the fragment carrying the CMV promoter and adjacent eGFP coding sequence. The resulting pVT313 backbone was then gel purified and ligated to a 1-0.24 kB BamHI/ClaI fragment (excised from pHi2-eGFP) carrying the CMV promoter operably linked to the Tat coding sequence. The resulting vector pVT1542 was then transformed into HT29 cells and selected to identify stable inserts. The nucleotide sequences encoding each presumptive cytotoxic/cytostatic agents were then cloned into an HIV2-based retroviral expression vector for testing in the Tat-modified HT29 cell line. Using techniques common to the field, the perturbagen insert was spliced into the EcoRI site of pVT1567. As a result of these manipulations each agent is attached to the C-terminus of a dead GFP (dGFP) scaffold that was, in turn, operably linked to an HIV2 promoter. When introduced into Tat-HT29 cell line, perturbagen expression is driven by the product of the Tat gene. As a result of these changes (in combination with the high MOI/high gene dosage promoted by Somata) transduced gene expression levels can reach as high as 30 μM.

Using Somata in combination with the cell-lethal assay and Tat-engineered HT29 cells, 3,840 independent clones from the cycle 4 sublibrary and a cycle 7 sublibrary that had been depleted of the two pro-apoptotic clones, were tested. The backgrounds in the assay were observed to be low and the reproducibility high, thus allowing individual clones with dead cell numbers that exceeded the mean by two standard deviations (σ) and/or clones with total cell numbers less than 2σ, to be easily identified as candidate cell-lethal clones. Using the procedures described above, one hundred and nineteen clones were confirmed as producing bona fide cytotoxic/cytostatic effects. When DNA sequences were obtained for these clones and analyzed, the number condensed to a total of 11 unique sequences. Sequence data showed that in addition to the two previous pro-apoptotic clones, 8 of 11 remaining sequences encoded products that were in-frame fusions of known or predicted native proteins.

Because of the high frequency of the pro-apoptotic clones and the concern of potential contamination by this strong cytotoxic agent, two methods were used to verify that individual clones were bona fide cell-lethals unrelated. First, PCR reactions with primers specific to the pro-apoptotic clones tested the possibility of low-level contamination. Secondly, each unique clone was re-transformed into E. coli, and two independent colonies were picked, sequenced, and tested in Somata. Experiments with 4 replicates of each clone provided values for the kill index of the clones where the term “kill index” is defined as a normalized ratio of dead/dying cells to total cells in each well.

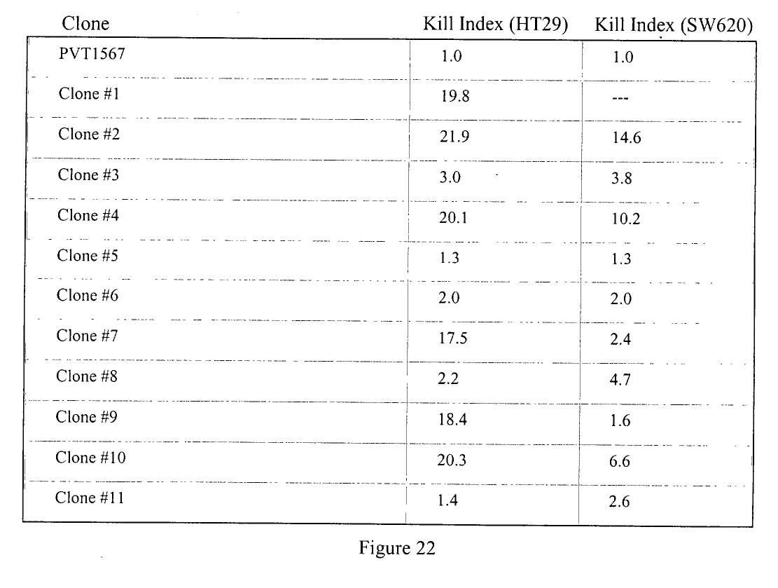

The kill index of the various cell-lethal clones varied from 20.3 to 1.3 (FIG. 22). The perturbagen having the most potent kill index was found to be the 120 a.a. polypeptide fragment. In contrast, another perturbagen had a kill index of roughly one, suggesting that this clone likely caused cell cycle arrest, rather than cell death.

Cell-Lethal Clones Function at the Protein Level

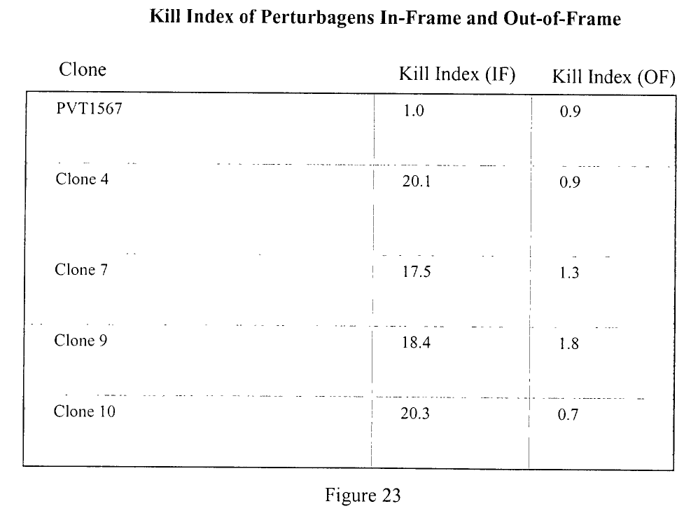

To begin to address mechanistic questions about the cell-lethal clones, the four most penetrant clones were examined for activity at the protein level. Expression constructs in which a stop codon was inserted between the GFP scaffold sequence and the cDNA, were tested in Somata side-by-side with the parental constructs. In every case the altered constructs were far less active than their parents (see FIG. 23). Northern blots confirmed that RNA expression in the constructs containing the added stop codon was roughly equivalent to RNA levels in cells transduced with parental constructs (data not shown). These experiments suggested that the activity of 5 cell-lethal clones resides in the protein, rather than RNA, expressed products.

Selectivity of Cytotoxic Clones

As a first step toward exploring phenotypic selectivity among the cell lethal clones, the cytotoxic effects of the perturbagens were tested in a second human colon adenocarcinoma line. Using techniques described previously, SW620 cells (ATCC # CCL227) were engineered to carry a Tat expression construct. Subsequently, the cells were infected with the P_{HIV2}-perturbagen vectors, and selected to identify stable integrants. Cells carrying the two constructs were then tested in the context of Somata assays to assess and compare the cytotoxicity of the clones in the SW620 and HT29 backgrounds. Kill indices for the set of clones revealed that two clones were remarkably selective in their cytotoxic effects. One clone had a kill index 11.5-fold higher in HT29 cells. Similarly, a second clone exhibited a kill index that was 7.3-fold higher HT29 cells than SW620 cells. Other clones had equivalent indices in both cell types or mild (e.g., 2-fold) preferences in one cell type or the other (FIG. 22).

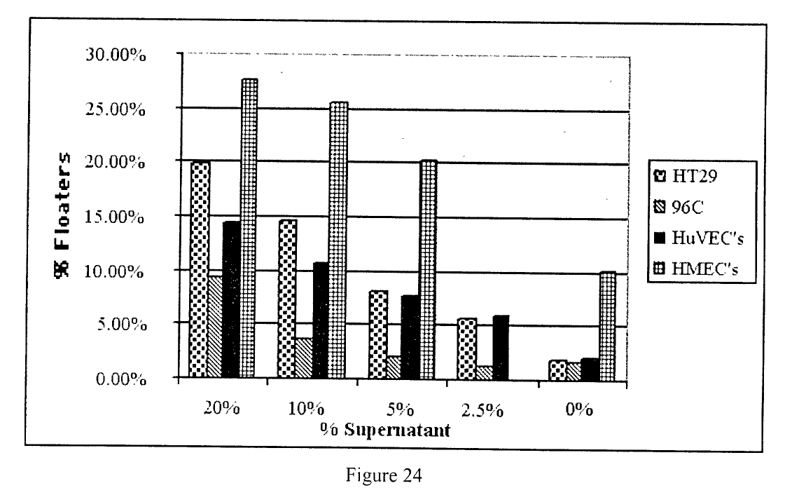

To further study the cytotoxic effects, one clone (clone 3) was reintroduced into multiple cell lines (HT29, HuVECs, HMECs, and 96C, an HMEC (Human Umbilical Vein Endothelial Cells,) line transformed with SV40 Lg T Ag, hTERT, and V12H-Ras,) and studied for it's ability to induce floaters. Unlike the control plasmid (pVT340) and mock infection experiments, which did not alter background levels of floaters (˜1-2% in HT29, HuVECs, and 96C cells, ˜10% in HMECs), Day 3 cultures containing this particular perturbagen exhibited large numbers of floaters under various different infection conditions (i.e. 20%, 10%, 5%, and 2.5% supernatants, see, FIG. 24). These results suggest that the clone induced a strong cytotoxic effect on a variety of cell types and warrants further analysis (e.g. target identification).

### Example Eleven

### HTS Trans-FACS Assay

Introduction

One non-limiting example of the application of these high-throughput procedures to TransFACS focuses on the identification of perturbagen sequences that are capable of modulating the β-catenin pathway. In the primary screen (see USPTO Application # 60/253,325), four rounds of enrichment were performed to isolate sequences capable of suppressing the expression of GFP from a TBE2-GFP reporter construct. Specifically, modified HEK293 cells (clone S4535) containing the pVT312-β-catenin S45Y expression vector, the pVT806 TBE2-EGFP reporter construct, and members of the cDNA (perturbagen) expression library were screened by FACS for perturbagen sequences that down-regulated the expression of GFP. After four successive rounds of enrichment, during which time the population was screened for “dim” cells (i.e. cells in which GFP expression had been down-regulate), individual clones were shuttled into the high throughput screen for further analysis.

HT Screen for Modulators of TBE2-GFP Expression in HEK293 Cells.

To test the ability of individual perturbagens to down-regulate the TBE2-EGFP reporter construct, three hundred S4535 cells (HEK293 cells containing both the pVT312-β-catenin S45Yconstruct and pVT806 TBE2-EGFP reporter) were plated in each well of a 96-well format and allowed to attach overnight. Subsequently, a single viral supernatant (85 ul) and polybrene (final concentration of 4 ug/ml) were added to each well and allowed to incubate for 16 hours. Following viral infection, the overlying media was replaced with 200 ul of fresh DMEM⁺. All of the above mentioned procedures were performed by hand or made use of the Sorval “Cytomat” and a Beckman “Multimek” instrumentation.

Perturbagen expressing S4535 cells were subsequently cultured for 6 days at 37° C. (5% CO₂) before being analyzed by FACS. To prepare the cells for analysis, each well was washed 1× with PBS and then treated with trypsin (50 ul of a 0.05% solution plus 53 mM EDTA, 10 minutes, 37° C., Life Technologies; Gaithersburg, Md.) to release the cells from the surface of the well. Subsequently, 150 ul of DMEM+10% FCS was added to each well to neutralize the trypsin. Samples were then analyzed on the FL1 channel of a Coulter Epics XL-MCL (Beckman Coulter; Fullerton, Calif.) using EXPO software and an automated 32-position sample carousel.

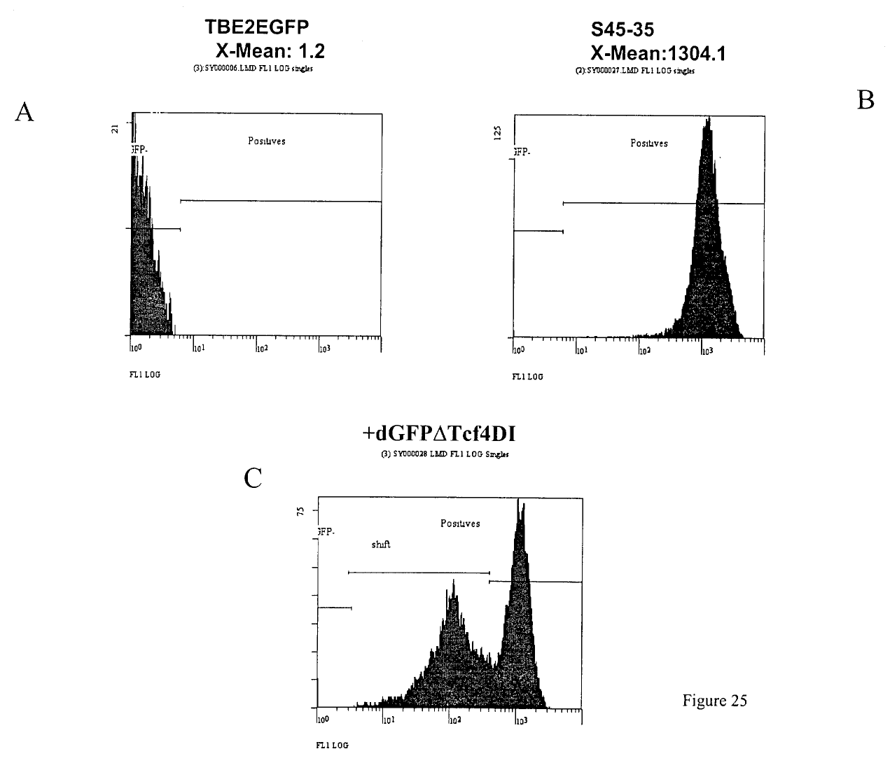

To set the position of the bright gate for this assay, the fluorescent signature of a population of S4535 cells containing pVT1515, the parental (control) plasmid used to construct the perturbagen expression library, was analyzed (FIG. 25). Introduction of the pVT1515 control plasmid into S4535 does not evoke a significant change in the GFP expression profile, and thus the boundaries of the bright cell gate were set to ensure that approximately 95% of a pVT1515-carrying S4535 population fell within the gate's borders. The position of the “Dim” gate was set using a synthetic dominant negative perturbagen, TcfΔ30 (FIG. 25). Previous studies have shown that in TcfΔ30-expressing S4535 populations, a large fraction of the cells down-regulate GFP expression to form a second, “dim” peak. Thus, the lower boundary of the bright gate and the position of the TcfΔ30-induced dim peak were used to set the margins of the dim gate.

Analysis of False Positive Rates

To determine the frequency of false positives in the high-throughput assay, ninety-six wells of S4535 cell were transduced with pVT1515. Following the 5-day period of incubation, cells were processed as described previously and analyzed to determine the level of GFP fluorescence. Of the ninety-six samples, ninety-four gave fluorescent profiles that were indistinguishable from cultured S4535 cells. Two wells showed small but statistically significant numbers of cells in the dim gate, suggesting the rate of false positives was approximately 2.13%.

Analysis of Perturbagen Encoding Clones.

Using the procedures described above, nine hundred and fifty seven clones (obtained after four rounds of TransFACS screening, en mass) were tested for their ability to alter GFP expression levels in the S4535 clonal line. Of the roughly 1,000 clones analyzed, about 8% (76 clones) were judged positive using the parameters previously described. When the DNA sequences of these clones were matched with the FACS data, all the positive clones corresponded with three previously sequence that are documented to have interactions with the β-catenin/APC/TCF4 pathway. The penetrance of all the clones were comparable and similar to TCF4Δ30.

### Example Twelve

### HTS Viral Assay

Introduction

One non-limiting application of Somata is to identify perturbagen sequences that are capable of inhibiting rhinoviral (RV 14) induced cell death. Prior to performing the HT procedures, four rounds of screening were performed during which a population of H1-HeLa cells containing a perturbagen expression library were sorted by FACS, en mass, for sequences capable of inhibiting RV-14 induced cell death (see U.S. Ser. No. 09/259,155). Specifically, H1-Hela cells (human cervical adenocarcinoma cells, ATCC CRL-1958) were plated in T175 flasks and simultaneously transduced with the retroviral supernatant containing the cDNA expression library (pVT 352.1-dead GFP-cDNA). After three days of growth to allow expression of the dead GFP-cDNA fusion constructs, cells were infected with a sufficient quantity of RV-14 determined to kill between 99 and 99.9% of the population. Cells were then cultured over the course of 3-5 days, during which time the culturing temperature, and media conditions were altered to minimize the possibility of secondary viral infections brought about by virus released from infected cells. Specifically, following the primary infection, culture incubation temperatures were raised from 33° C. to the non-permissive temperature of 39° C., and a monoclonal antibody capable of binding and neutralizing newly released virion particles, was added to each culture (mAb 17, a gift of T.Smith, Purdue University, West Lafayette Ind., see Smith, T. J. et al. (1996) “Neutralizing antibody to human rhinovirus 14 penetrates the receptor-binding canyon.”Nature 383(6598): 350-4.) Subsequently, live, adherent cells capable of surviving in the presence of RV14 were collected, and the perturbagen encoding sequences were retrieved, packaged, and tested in the HTS system.

HTS Viral Perturbagen Screen

To test the ability of individual perturbagens to protect cells from RV-14 induced cell death, 2,000 H1-HeLa cells (DMEM⁺, Gibco BRL) are plated in each well of a 96-well format and allowed to attach over the course of several hours (33° C., 5% CO₂). Subsequently, each well is transduced with a retrovirus containing a unique, perturbagen-encoding sequence derived from the previous viral screen (performed en mass). As described previously, this can be accomplished by adding 85 ul of a viral supernatant and polybrene (final concentration of 4 ug/ml) to each well and allowing the mixture to incubate for 16 hours. Following a media change, the cells are then incubated for an additional four days at 33° C. The media is then replaced with a low serum media (2% FCS), and the cells are challenged with a quantity of RV-14 (ATCC VR-284) that is sufficient to kill 99% of the H1-HeLa cells (MOI (multiplicity of infection)=10-50). To obtain stocks of rhinoviral supernatants of sufficient MOI these procedures, sub-confluent plates of HeLa cells growing at 33° C. are infected with RV-14 in the presence of 2% serum and allowed to propagate until >95% cell death is observed (˜3-7 days). Subsequently, the cells and the media are collected, freeze/thawed two times at −80° C., and centrifuged at 1200-× g to remove cellular debris. This viral stock is then stored at −80° C. in 5 ml aliquots. Virus thawed for use is kept at 4° C. for up to one month.

Titering viral supernatants for HT assays is accomplished by determining the TCID₅₀ (Tissue culture infectious dose necessary for 50% of cultures to be infected, see, for example Reed and Muench, Am. J. Hyg., vol. 27, pages 493-497 (1938), or U.S. Pat. No. 6,127,422). Specifically, serial 10-fold dilutions of RV-14 viral supernatants are added to rows of a 96 well microtiter plate that have been seeded the day before with 2000 HeLa cells per well. The virus and HeLa cells are then incubated for seven days, upon which time individual wells are scored for infection either by microscopic examination or by fixation with methanol and staining with crystal violet.

Returning to the HT viral screen, following a four-hour incubation of RV14 with the perturbagen-expressing H1-HeLa cells, the media is removed and replaced with fresh DMEM supplemented with a neutralizing monoclonal antibody mAb17 (final concentration=0.1% total volume). Cultures are then incubated for an additional 48 hours before being stained with a vital dye. Optionally, all, or some portion of the two-day incubation can be performed at an elevated temperature (39° C.) that is permissive for H1-HeLa cell growth, but prevents secondary infections by RV-14. Cells can be stained with a variety of reagents including calcein, propidium iodide, Sytox® (Molecular Probes), and others. When staining with calcein, the media is removed and replaced with 200 ul of a 1 uM calcein solution in PBS into each well. The plates are then incubated for thirty additional minutes before being analyzed on a SpectaMax Gemini XS plate reader (Molecular Dynamics). All steps up to and including transduction of the retrovirus into H1-HeLa cells are performed using the previously described robotics (Sorval “Cytomat” and a Beckman “Multimek” instrumentation). To minimize the possibility of RV-14 contamination of the equipment, all steps that involved manipulation of RV-14 are performed either by hand (8-channel pipette) or utilize HTS machines that are dedicated to infectious viral particles.

All references cited within the body of the instant specification are hereby incorporated by reference in their entirety.

What is claimed is:

1. A method to identify a candidate compound that inhibits cell proliferation comprising the steps of:
a) introducing into a cell population a polynucleotide library under conditions that permit expression of polypeptides and/or ribonucleic acid encoded by the library polynucleotides;
b) isolating cells in the population in which proliferation is inhibited; and
c) isolating the polynucleotide library sequence(s) from cells detected in step (b), wherein inhibited proliferation of the cell identifies the encoded polypeptide or ribonucleic acid as a candidate compound that inhibits cell proliferation.

2. The method of claim 1 wherein the cells in step (b) are dead and/or dying.

3. The method of claim 2 wherein the cells are apoptotic.

4. The method of claim 1 wherein the cells in step (b) are growth arrested.

5. The method of claim 2 or 3 wherein cells isolated in step (b) are identified by a loss of adherence to a solid support.

6. The method of claim 2 or 3 wherein cells isolated in step (b) are detected using an agent that recognizes and binds an intracellular component that is accessible in dead and/or dying cells or is activated during cell cycle arrest or programmed cell death.

7. The method of claim 6 wherein the dead and/or dying cells are apoptotic.

8. The method of claim 6 wherein the agent is a labeling compound.

9. The method of claim 8 wherein the labeling compound binds a membrane lipid.

10. The method of claim 8 wherein the labeling compound is a DNA affinity dye.

11. The method of claim 6 wherein the agent is an antibody; said antibody being immunospecific for an intracellular antigen.

12. The method of claim 11 wherein the antibody is detectably labeled.

13. The method of claim 1 wherein the cells in step (b) are isolated using fluorescent activated cell sorting (FACS).

14. The method of any one of claims 8 through 12 wherein cells in step (b) are detected using fluorescent activated cell sorting (FACS).

15. The method of claim 1 wherein steps (a) and (b) are automated.
